SEQUENCE LISTING (31 sequences)

SEQ ID NO: 1 - DNA, 8 bp, Artificial Sequence
  Nucleotide sequence of the variant sgp2/CS of subgenomic promoter
gttagggt

SEQ ID NO: 2 - DNA, 45 bp, Artificial Sequence
  Sequence of the variant sgp2/45 of subgenomic promoter 2
cgcattgagggtgttagggtaaccagcatcagtgaagagaaacaa

SEQ ID NO: 3 - DNA, 55 bp, Artificial Sequence
  Sequence of the variant sgp2/55 of subgenomic promoter 2
cgcattgagggtgttagggtaaccagcatcagtgaagagaaacaacccacctcaa

SEQ ID NO: 4 - DNA, 8 bp, Artificial Sequence
  Nucleotide sequence of the variant sgp2/DS of subgenomic promoter
tggcaaca

SEQ ID NO: 5 - DNA, 90 bp, Artificial Sequence
  Sequence of the variant sgp2/90 of subgenomic promoter 2
cgcattgagggtgttagggtaaccagcatcagtgaagagaaacaacccacctcaagtgtgacctcatcatttcaggacacaatggcaaca

SEQ ID NO: 6 - DNA, 101 bp, Artificial Sequence
  Nucleotide sequence of the variant sgp2/101 of subgenomic promoter 2
cactcaacgaccgcattgagggtgttagggtaaccagcatcagtgaagagaaacaacccacctcaagtgtgacctcatcatttcaggacacaatggcaaca

SEQ ID NO: 7 - DNA, 170 bp, Artificial Sequence
  Sequence of the variant sgp2/170 of subgenomic promoter 2
cactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcgagtgtgacctcatcatttcaggacaca

SEQ ID NO: 8 - DNA, 6226 bp, Artificial Sequence
  Taiwanese FoMV isolate pCF, with no modifications
gaaaactcttccgaaaccgaaactgactgaaactacctcgaccgaccttagaacccaagaaccctacgggtgcggccactatgtctatcgaggcagttttcgaccaggttaccgacccatcgctccgcgctgtgattcaggaggaagcgcacaaacagatcaaagatttatttaaggaaacgacgcgctgcaatccctacgccataccgcaagctgggcgcaaggttttggagaagtacgccatcccctacaacccgtactctctcaaactacaccctcacgcagcctcaaaagcgtttgaagtgtcgctctacgaggctgcatctaactacctcccctccacctcctcaactcccgtcacattcatgttcacaaaaccgggcaagctcagattcttcaggcgccgaggtcacgtggacaaattcgttaatgctgacatagttccaagagacttggctagatacccacgcgacacagtctacagttatctgcccgagatcaccaccacacacgctttcattggcgacaccctacatcacttcggtgaggactttctcgtcgaggttttctccaggtcaccgaaactagaagtgcttctagccaccatggtattaccacccgaagccttttacagcatggagtcccttcatccctcggtttacactctcctctacagggacgaccgattcctatacctgcctggtggcctgtctggcggtgagtacgaacatcgctataaggacctaaactggctaacatttggcacagttacgcacggcgggatcactatcacaggagaacgcattgagactaaggccgcgaatcatcttttcctcttcagaagagggcgactggcgacaccaaaattccgctcattcgatatgcccgagcctatggtcctgcttcccaaggttttccgccccgcaaagtacaatgtgcagaagccgattccccgggagaaagcaaacaaatggttgatgtacgttaaatccatcggcaatgccaccattcgtgacgtatgggctaagctgaggcaaaccatagccaatgcagacattggactcttctcgcccacggagctcgtgcatctcacgaattacttcctgctcctgggccggcttgactcacacaactccttcgaccaagtactggccgacagtgtgctgaaagcatggttcagaccaatggtcgcgaagcttcaggagatcaagcacaaactcatggggcagacccaattcatgcaactctgccaagcgctagagatgacggaggtggacctagtctttgaggtccgggactccaagactccccacaaacaagctgtgccgttggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcccgagagctctagctcgcgtgaaattgagcaccaaccggcgcctgagatcacgcttgacgcggaagaacctcagcgagacgatctgccttgggacgcttggaaaacacaattgagggcgcttggcttcgaggcctccgaaaggcagtatgacccggacggtgaactgatctctcctatcctgagcacccgaaggttgcctaaaactcccatagacacaacactctacgccacgctagacaagattgcacgctgcccaactttctacaagcctgacacagatcgcgcgcagacttacgctcgcgatgtcatggcggggaaaaccggtgccattctcaagcaacaaccctttgagtggaaaaccacgctcaagcgcaaaactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacgacctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaggaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagcttcttccagaaggttgaagcaacaccgtacctcaaagccatcctcaacctcaacaaagagattaaagtcactgagggtgacttgacagaagaaccgccgagggagcccgctcctcccaccacgcacctgcctgttgaaaacagaattattcttaatgaggccctagtcgaaccgctgcccgacaaatatgaccgcgagatctactccaactccactggcttttcaaactgcatacagactcaagacccgtacatccaagccttccaacatcagcaagccaaggacgagacattgttctgggcaaccgtcgagaagaggctcgcagcatctacgccgaaggacaactggacagaattcaggactaagagacctctgggtgacgtgctttggctcgcgtacaagcgggcgatggagctcccagatgagcccatcaagtttaacccagagctctggtgggcatgtgcagatgaggtgcaaaagacctacctctccaagcccatacacgcactcaagaacggaattcttcggcagtcacccgactttgactggaacaaactgcagattttcctcaagtcacagtgggttaagaaaattgacaaaatcgggaaaattgacgtcaacgctggacagacaattgccgcattttaccaaccaaccgttatgctgtttggaaccatggcgagatacatgcgccgcatccgcgacacttatcaacccggcgacatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtactgaaagccctgcaccatgatatccctcatgaggttgtggaagcctacgtagccctcaaactcaactcaaaaatgtttctgggcacactggcgattatgagattaactggtgagggacccaccttcgacgctaacacagagtgcaacatcgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttgctacctacatacgagctaggtgatgcactctacgaaatcttcgacgagaggcaaatgaacgcgcactatcagtcggtcaggacgcttatcacatgcgcccacaccaaagtcctccgagttgcacaggcacttcaggaagactgcaccctctttagctccatctaacaggttttgagttaggctaactccactgacgaattaaataacaatggatagtgaaatagttgaacgactaacaaagcttggtttcgtcaaaacttcacacacgcacatcgctggcgagcccctcgtgatccacgctgttgctggggccggtaaaaccaccctccttcggtccctacttgaattacctggagtggaagtcttcacaggcggggagcacgatcctccaaacttgtcagggaaatatatccgctgtgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgttgacgctttgcgtagggttggtttcgacatcacctttgccggcacgcagactgaagactacggattccaggaaggccatctctacactagtcaattttacggacaggtcatttcacttgacacgcaggcccataagattgctgtgcgccacggacttgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctcgtctttttcatgctcacacgcaacaatctgccacacgttggtgataacatccactcactaccccacggaggcagttacattgacggtaccaagtccatcaactaccgcccacctgcgtcacgctacccctcatctaacttattcgctttcgctccaccaatactcgccgcggtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccagcatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacgatcaaacaaggcgcccagtgacgaggagttggtacgcatcatcaacgcggcgcagaagcgaggcctcacacccgcggcctttgttcaagcagctataatcttcaccatggaatccatggacaagggcgccaccgactccacgattttcacgggaaaacacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagatcttcaagaaggacaatttgagtcaggcagcatccagaaaccaactcctaggaacgcaagcctccatcacgcgcgggagactcaacggcgcaccagcactaccaaacaatgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaaaaaaaaaaaaaaaaaaaaaaaaaaa

SEQ ID NO: 9 - DNA, 6201 bp, Artificial Sequence
  Wheat-adapted isolate PV139wa, with no modifications
gaaaactcttccgaaaccgaaactgactgaaactacctcgaccgaccttagaacccaagaacccaacgggtgcggccactatgtctatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggccgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtattggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctccgaaaggcagtatgacccggacggtgaactgatctctcctatcctgagcacccgaaggttgcctaaaactcccataaacacaacactctacgccacgctagacaagattgcacgctgcccaactttctacaagcctgacacagatcgcgcgcagacttacgctcgcgacgtcatggcggggaaaaccggtgccattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggtcaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctgcctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgcgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactcttcttcctcacacagccatatctagccaccagacgatccaggtgcgttcggtgcttcgttgtccacggggcatgcacgaatcacacttagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctacaccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaa

SEQ ID NO: 10 - DNA, 7013 bp, Artificial Sequence
  Sequence of the vector pCF.sgp2/45-GFP where the duplicated sgp2 is 45 nt-long
gaaaactcttccgaaaccgaaactgactgaaactacctcgaccgaccttagaacccaagaaccctacgggtgcggccactatgtctatcgaggcagttttcgaccaggttaccgacccatcgctccgcgctgtgattcaggaggaagcgcacaaacagatcaaagatttatttaaggaaacgacgcgctgcaatccctacgccataccgcaagctgggcgcaaggttttggagaagtacgccatcccctacaacccgtactctctcaaactacaccctcacgcagcctcaaaagcgtttgaagtgtcgctctacgaggctgcatctaactacctcccctccacctcctcaactcccgtcacattcatgttcacaaaaccgggcaagctcagattcttcaggcgccgaggtcacgtggacaaattcgttaatgctgacatagttccaagagacttggctagatacccacgcgacacagtctacagttatctgcccgagatcaccaccacacacgctttcattggcgacaccctacatcacttcggtgaggactttctcgtcgaggttttctccaggtcaccgaaactagaagtgcttctagccaccatggtattaccacccgaagccttttacagcatggagtcccttcatccctcggtttacactctcctctacagggacgaccgattcctatacctgcctggtggcctgtctggcggtgagtacgaacatcgctataaggacctaaactggctaacatttggcacagttacgcacggcgggatcactatcacaggagaacgcattgagactaaggccgcgaatcatcttttcctcttcagaagagggcgactggcgacaccaaaattccgctcattcgatatgcccgagcctatggtcctgcttcccaaggttttccgccccgcaaagtacaatgtgcagaagccgattccccgggagaaagcaaacaaatggttgatgtacgttaaatccatcggcaatgccaccattcgtgacgtatgggctaagctgaggcaaaccatagccaatgcagacattggactcttctcgcccacggagctcgtgcatctcacgaattacttcctgctcctgggccggcttgactcacacaactccttcgaccaagtactggccgacagtgtgctgaaagcatggttcagaccaatggtcgcgaagcttcaggagatcaagcacaaactcatggggcagacccaattcatgcaactctgccaagcgctagagatgacggaggtggacctagtctttgaggtccgggactccaagactccccacaaacaagctgtgccgttggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcccgagagctctagctcgcgtgaaattgagcaccaaccggcgcctgagatcacgcttgacgcggaagaacctcagcgagacgatctgccttgggacgcttggaaaacacaattgagggcgcttggcttcgaggcctccgaaaggcagtatgacccggacggtgaactgatctctcctatcctgagcacccgaaggttgcctaaaactcccatagacacaacactctacgccacgctagacaagattgcacgctgcccaactttctacaagcctgacacagatcgcgcgcagacttacgctcgcgatgtcatggcggggaaaaccggtgccattctcaagcaacaaccctttgagtggaaaaccacgctcaagcgcaaaactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacgacctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaggaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagcttcttccagaaggttgaagcaacaccgtacctcaaagccatcctcaacctcaacaaagagattaaagtcactgagggtgacttgacagaagaaccgccgagggagcccgctcctcccaccacgcacctgcctgttgaaaacagaattattcttaatgaggccctagtcgaaccgctgcccgacaaatatgaccgcgagatctactccaactccactggcttttcaaactgcatacagactcaagacccgtacatccaagccttccaacatcagcaagccaaggacgagacattgttctgggcaaccgtcgagaagaggctcgcagcatctacgccgaaggacaactggacagaattcaggactaagagacctctgggtgacgtgctttggctcgcgtacaagcgggcgatggagctcccagatgagcccatcaagtttaacccagagctctggtgggcatgtgcagatgaggtgcaaaagacctacctctccaagcccatacacgcactcaagaacggaattcttcggcagtcacccgactttgactggaacaaactgcagattttcctcaagtcacagtgggttaagaaaattgacaaaatcgggaaaattgacgtcaacgctggacagacaattgccgcattttaccaaccaaccgttatgctgtttggaaccatggcgagatacatgcgccgcatccgcgacacttatcaacccggcgacatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtactgaaagccctgcaccatgatatccctcatgaggttgtggaagcctacgtagccctcaaactcaactcaaaaatgtttctgggcacactggcgattatgagattaactggtgagggacccaccttcgacgctaacacagagtgcaacatcgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttgctacctacatacgagctaggtgatgcactctacgaaatcttcgacgagaggcaaatgaacgcgcactatcagtcggtcaggacgcttatcacatgcgcccacaccaaagtcctccgagttgcacaggcacttcaggaagactgcaccctctttagctccatctaacaggttttgagttaggctaactccactgacgaattaaataacaatggatagtgaaatagttgaacgactaacaaagcttggtttcgtcaaaacttcacacacgcacatcgctggcgagcccctcgtgatccacgctgttgctggggccggtaaaaccaccctccttcggtccctacttgaattacctggagtggaagtcttcacaggcggggagcacgatcctccaaacttgtcagggaaatatatccgctgtgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgttgacgctttgcgtagggttggtttcgacatcacctttgccggcacgcagactgaagactacggattccaggaaggccatctctacactagtcaattttacggacaggtcatttcacttgacacgcaggcccataagattgctgtgcgccacggacttgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctcgtctttttcatgctcacacgcaacaatctgccacacgttggtgataacatccactcactaccccacggaggcagttacattgacggtaccaagtccatcaactaccgcccacctgcgtcacgctacccctcatctaacttattcgctttcgctccaccaatactcgccgcggtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccagcatcagtgaagagaaacaagtcgacagctatcgatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaatctagacgcattgagggtgttagggtaaccagcatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacgatcaaacaaggcgcccagtgacgaggagttggtacgcatcatcaacgcggcgcagaagcgaggcctcacacccgcggcctttgttcaagcagctataatcttcaccatggaatccatggacaagggcgccaccgactccacgattttcacgggaaaacacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagatcttcaagaaggacaatttgagtcaggcagcatccagaaaccaactcctaggaacgcaagcctccatcacgcgcgggagactcaacggcgcaccagcactaccaaacaatgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaaaaaaaaaaaaaaaaaaaaaaaaaaa

SEQ ID NO: 11 - DNA, 7023 bp, Artificial Sequence
  Sequence of the vector pCF.sgp2/55-GFP where the duplicated sgp2 is 55 nt-long
gaaaactcttccgaaaccgaaactgactgaaactacctcgaccgaccttagaacccaagaaccctacgggtgcggccactatgtctatcgaggcagttttcgaccaggttaccgacccatcgctccgcgctgtgattcaggaggaagcgcacaaacagatcaaagatttatttaaggaaacgacgcgctgcaatccctacgccataccgcaagctgggcgcaaggttttggagaagtacgccatcccctacaacccgtactctctcaaactacaccctcacgcagcctcaaaagcgtttgaagtgtcgctctacgaggctgcatctaactacctcccctccacctcctcaactcccgtcacattcatgttcacaaaaccgggcaagctcagattcttcaggcgccgaggtcacgtggacaaattcgttaatgctgacatagttccaagagacttggctagatacccacgcgacacagtctacagttatctgcccgagatcaccaccacacacgctttcattggcgacaccctacatcacttcggtgaggactttctcgtcgaggttttctccaggtcaccgaaactagaagtgcttctagccaccatggtattaccacccgaagccttttacagcatggagtcccttcatccctcggtttacactctcctctacagggacgaccgattcctatacctgcctggtggcctgtctggcggtgagtacgaacatcgctataaggacctaaactggctaacatttggcacagttacgcacggcgggatcactatcacaggagaacgcattgagactaaggccgcgaatcatcttttcctcttcagaagagggcgactggcgacaccaaaattccgctcattcgatatgcccgagcctatggtcctgcttcccaaggttttccgccccgcaaagtacaatgtgcagaagccgattccccgggagaaagcaaacaaatggttgatgtacgttaaatccatcggcaatgccaccattcgtgacgtatgggctaagctgaggcaaaccatagccaatgcagacattggactcttctcgcccacggagctcgtgcatctcacgaattacttcctgctcctgggccggcttgactcacacaactccttcgaccaagtactggccgacagtgtgctgaaagcatggttcagaccaatggtcgcgaagcttcaggagatcaagcacaaactcatggggcagacccaattcatgcaactctgccaagcgctagagatgacggaggtggacctagtctttgaggtccgggactccaagactccccacaaacaagctgtgccgttggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcccgagagctctagctcgcgtgaaattgagcaccaaccggcgcctgagatcacgcttgacgcggaagaacctcagcgagacgatctgccttgggacgcttggaaaacacaattgagggcgcttggcttcgaggcctccgaaaggcagtatgacccggacggtgaactgatctctcctatcctgagcacccgaaggttgcctaaaactcccatagacacaacactctacgccacgctagacaagattgcacgctgcccaactttctacaagcctgacacagatcgcgcgcagacttacgctcgcgatgtcatggcggggaaaaccggtgccattctcaagcaacaaccctttgagtggaaaaccacgctcaagcgcaaaactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacgacctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaggaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagcttcttccagaaggttgaagcaacaccgtacctcaaagccatcctcaacctcaacaaagagattaaagtcactgagggtgacttgacagaagaaccgccgagggagcccgctcctcccaccacgcacctgcctgttgaaaacagaattattcttaatgaggccctagtcgaaccgctgcccgacaaatatgaccgcgagatctactccaactccactggcttttcaaactgcatacagactcaagacccgtacatccaagccttccaacatcagcaagccaaggacgagacattgttctgggcaaccgtcgagaagaggctcgcagcatctacgccgaaggacaactggacagaattcaggactaagagacctctgggtgacgtgctttggctcgcgtacaagcgggcgatggagctcccagatgagcccatcaagtttaacccagagctctggtgggcatgtgcagatgaggtgcaaaagacctacctctccaagcccatacacgcactcaagaacggaattcttcggcagtcacccgactttgactggaacaaactgcagattttcctcaagtcacagtgggttaagaaaattgacaaaatcgggaaaattgacgtcaacgctggacagacaattgccgcattttaccaaccaaccgttatgctgtttggaaccatggcgagatacatgcgccgcatccgcgacacttatcaacccggcgacatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtactgaaagccctgcaccatgatatccctcatgaggttgtggaagcctacgtagccctcaaactcaactcaaaaatgtttctgggcacactggcgattatgagattaactggtgagggacccaccttcgacgctaacacagagtgcaacatcgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttgctacctacatacgagctaggtgatgcactctacgaaatcttcgacgagaggcaaatgaacgcgcactatcagtcggtcaggacgcttatcacatgcgcccacaccaaagtcctccgagttgcacaggcacttcaggaagactgcaccctctttagctccatctaacaggttttgagttaggctaactccactgacgaattaaataacaatggatagtgaaatagttgaacgactaacaaagcttggtttcgtcaaaacttcacacacgcacatcgctggcgagcccctcgtgatccacgctgttgctggggccggtaaaaccaccctccttcggtccctacttgaattacctggagtggaagtcttcacaggcggggagcacgatcctccaaacttgtcagggaaatatatccgctgtgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgttgacgctttgcgtagggttggtttcgacatcacctttgccggcacgcagactgaagactacggattccaggaaggccatctctacactagtcaattttacggacaggtcatttcacttgacacgcaggcccataagattgctgtgcgccacggacttgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctcgtctttttcatgctcacacgcaacaatctgccacacgttggtgataacatccactcactaccccacggaggcagttacattgacggtaccaagtccatcaactaccgcccacctgcgtcacgctacccctcatctaacttattcgctttcgctccaccaatactcgccgcggtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccagcatcagtgaagagaaacaacccacctcaagtcgacagctatcgatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaatctagacgcattgagggtgttagggtaaccagcatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacgatcaaacaaggcgcccagtgacgaggagttggtacgcatcatcaacgcggcgcagaagcgaggcctcacacccgcggcctttgttcaagcagctataatcttcaccatggaatccatggacaagggcgccaccgactccacgattttcacgggaaaacacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagatcttcaagaaggacaatttgagtcaggcagcatccagaaaccaactcctaggaacgcaagcctccatcacgcgcgggagactcaacggcgcaccagcactaccaaacaatgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaaaaaaaaaaaaaaaaaaaaaaaaaaa

SEQ ID NO: 12 - DNA, 7058 bp, Artificial Sequence
  Sequence of the vector pCF.sgp2/90-GFP where the duplicated sgp2 is 90 nt-long . The start codon which is present in the duplicated region was mutated by a T to G substitution in the first repeat of the duplicated sequence
gaaaactcttccgaaaccgaaactgactgaaactacctcgaccgaccttagaacccaagaaccctacgggtgcggccactatgtctatcgaggcagttttcgaccaggttaccgacccatcgctccgcgctgtgattcaggaggaagcgcacaaacagatcaaagatttatttaaggaaacgacgcgctgcaatccctacgccataccgcaagctgggcgcaaggttttggagaagtacgccatcccctacaacccgtactctctcaaactacaccctcacgcagcctcaaaagcgtttgaagtgtcgctctacgaggctgcatctaactacctcccctccacctcctcaactcccgtcacattcatgttcacaaaaccgggcaagctcagattcttcaggcgccgaggtcacgtggacaaattcgttaatgctgacatagttccaagagacttggctagatacccacgcgacacagtctacagttatctgcccgagatcaccaccacacacgctttcattggcgacaccctacatcacttcggtgaggactttctcgtcgaggttttctccaggtcaccgaaactagaagtgcttctagccaccatggtattaccacccgaagccttttacagcatggagtcccttcatccctcggtttacactctcctctacagggacgaccgattcctatacctgcctggtggcctgtctggcggtgagtacgaacatcgctataaggacctaaactggctaacatttggcacagttacgcacggcgggatcactatcacaggagaacgcattgagactaaggccgcgaatcatcttttcctcttcagaagagggcgactggcgacaccaaaattccgctcattcgatatgcccgagcctatggtcctgcttcccaaggttttccgccccgcaaagtacaatgtgcagaagccgattccccgggagaaagcaaacaaatggttgatgtacgttaaatccatcggcaatgccaccattcgtgacgtatgggctaagctgaggcaaaccatagccaatgcagacattggactcttctcgcccacggagctcgtgcatctcacgaattacttcctgctcctgggccggcttgactcacacaactccttcgaccaagtactggccgacagtgtgctgaaagcatggttcagaccaatggtcgcgaagcttcaggagatcaagcacaaactcatggggcagacccaattcatgcaactctgccaagcgctagagatgacggaggtggacctagtctttgaggtccgggactccaagactccccacaaacaagctgtgccgttggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcccgagagctctagctcgcgtgaaattgagcaccaaccggcgcctgagatcacgcttgacgcggaagaacctcagcgagacgatctgccttgggacgcttggaaaacacaattgagggcgcttggcttcgaggcctccgaaaggcagtatgacccggacggtgaactgatctctcctatcctgagcacccgaaggttgcctaaaactcccatagacacaacactctacgccacgctagacaagattgcacgctgcccaactttctacaagcctgacacagatcgcgcgcagacttacgctcgcgatgtcatggcggggaaaaccggtgccattctcaagcaacaaccctttgagtggaaaaccacgctcaagcgcaaaactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacgacctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaggaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagcttcttccagaaggttgaagcaacaccgtacctcaaagccatcctcaacctcaacaaagagattaaagtcactgagggtgacttgacagaagaaccgccgagggagcccgctcctcccaccacgcacctgcctgttgaaaacagaattattcttaatgaggccctagtcgaaccgctgcccgacaaatatgaccgcgagatctactccaactccactggcttttcaaactgcatacagactcaagacccgtacatccaagccttccaacatcagcaagccaaggacgagacattgttctgggcaaccgtcgagaagaggctcgcagcatctacgccgaaggacaactggacagaattcaggactaagagacctctgggtgacgtgctttggctcgcgtacaagcgggcgatggagctcccagatgagcccatcaagtttaacccagagctctggtgggcatgtgcagatgaggtgcaaaagacctacctctccaagcccatacacgcactcaagaacggaattcttcggcagtcacccgactttgactggaacaaactgcagattttcctcaagtcacagtgggttaagaaaattgacaaaatcgggaaaattgacgtcaacgctggacagacaattgccgcattttaccaaccaaccgttatgctgtttggaaccatggcgagatacatgcgccgcatccgcgacacttatcaacccggcgacatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtactgaaagccctgcaccatgatatccctcatgaggttgtggaagcctacgtagccctcaaactcaactcaaaaatgtttctgggcacactggcgattatgagattaactggtgagggacccaccttcgacgctaacacagagtgcaacatcgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttgctacctacatacgagctaggtgatgcactctacgaaatcttcgacgagaggcaaatgaacgcgcactatcagtcggtcaggacgcttatcacatgcgcccacaccaaagtcctccgagttgcacaggcacttcaggaagactgcaccctctttagctccatctaacaggttttgagttaggctaactccactgacgaattaaataacaatggatagtgaaatagttgaacgactaacaaagcttggtttcgtcaaaacttcacacacgcacatcgctggcgagcccctcgtgatccacgctgttgctggggccggtaaaaccaccctccttcggtccctacttgaattacctggagtggaagtcttcacaggcggggagcacgatcctccaaacttgtcagggaaatatatccgctgtgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgttgacgctttgcgtagggttggtttcgacatcacctttgccggcacgcagactgaagactacggattccaggaaggccatctctacactagtcaattttacggacaggtcatttcacttgacacgcaggcccataagattgctgtgcgccacggacttgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctcgtctttttcatgctcacacgcaacaatctgccacacgttggtgataacatccactcactaccccacggaggcagttacattgacggtaccaagtccatcaactaccgcccacctgcgtcacgctacccctcatctaacttattcgctttcgctccaccaatactcgccgcggtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccagcatcagtgaagagaaacaacccacctcaagtgtgacctcatcatttcaggacacaagggcaacagtcgacagctatcgatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaatctagacgcattgagggtgttagggtaaccagcatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacgatcaaacaaggcgcccagtgacgaggagttggtacgcatcatcaacgcggcgcagaagcgaggcctcacacccgcggcctttgttcaagcagctataatcttcaccatggaatccatggacaagggcgccaccgactccacgattttcacgggaaaacacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagatcttcaagaaggacaatttgagtcaggcagcatccagaaaccaactcctaggaacgcaagcctccatcacgcgcgggagactcaacggcgcaccagcactaccaaacaatgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaaaaaaaaaaaaaaaaaaaaaaaaaaa

SEQ ID NO: 13 - DNA, 7069 bp, Artificial Sequence
  Sequence of the vector pCF.sgp2/101-GFP where the duplicated sgp2 is 101 nt-long. The start codon which is present in the duplicated region was mutated by a T to G
gaaaactcttccgaaaccgaaactgactgaaactacctcgaccgaccttagaacccaagaaccctacgggtgcggccactatgtctatcgaggcagttttcgaccaggttaccgacccatcgctccgcgctgtgattcaggaggaagcgcacaaacagatcaaagatttatttaaggaaacgacgcgctgcaatccctacgccataccgcaagctgggcgcaaggttttggagaagtacgccatcccctacaacccgtactctctcaaactacaccctcacgcagcctcaaaagcgtttgaagtgtcgctctacgaggctgcatctaactacctcccctccacctcctcaactcccgtcacattcatgttcacaaaaccgggcaagctcagattcttcaggcgccgaggtcacgtggacaaattcgttaatgctgacatagttccaagagacttggctagatacccacgcgacacagtctacagttatctgcccgagatcaccaccacacacgctttcattggcgacaccctacatcacttcggtgaggactttctcgtcgaggttttctccaggtcaccgaaactagaagtgcttctagccaccatggtattaccacccgaagccttttacagcatggagtcccttcatccctcggtttacactctcctctacagggacgaccgattcctatacctgcctggtggcctgtctggcggtgagtacgaacatcgctataaggacctaaactggctaacatttggcacagttacgcacggcgggatcactatcacaggagaacgcattgagactaaggccgcgaatcatcttttcctcttcagaagagggcgactggcgacaccaaaattccgctcattcgatatgcccgagcctatggtcctgcttcccaaggttttccgccccgcaaagtacaatgtgcagaagccgattccccgggagaaagcaaacaaatggttgatgtacgttaaatccatcggcaatgccaccattcgtgacgtatgggctaagctgaggcaaaccatagccaatgcagacattggactcttctcgcccacggagctcgtgcatctcacgaattacttcctgctcctgggccggcttgactcacacaactccttcgaccaagtactggccgacagtgtgctgaaagcatggttcagaccaatggtcgcgaagcttcaggagatcaagcacaaactcatggggcagacccaattcatgcaactctgccaagcgctagagatgacggaggtggacctagtctttgaggtccgggactccaagactccccacaaacaagctgtgccgttggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcccgagagctctagctcgcgtgaaattgagcaccaaccggcgcctgagatcacgcttgacgcggaagaacctcagcgagacgatctgccttgggacgcttggaaaacacaattgagggcgcttggcttcgaggcctccgaaaggcagtatgacccggacggtgaactgatctctcctatcctgagcacccgaaggttgcctaaaactcccatagacacaacactctacgccacgctagacaagattgcacgctgcccaactttctacaagcctgacacagatcgcgcgcagacttacgctcgcgatgtcatggcggggaaaaccggtgccattctcaagcaacaaccctttgagtggaaaaccacgctcaagcgcaaaactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacgacctcgcccccgcctcagtggtcttcggcaagttctctaggtaccacataaatgccacacaccgcaaccccagaaacttggcaaacgccctcggtgtttactccgagacgcccggggaggttaaagtgctttacacgaggaacatcaggaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagcttcttccagaaggttgaagcaacaccgtacctcaaagccatcctcaacctcaacaaagagattaaagtcactgagggtgacttgacagaagaaccgccgagggagcccgctcctcccaccacgcacctgcctgttgaaaacagaattattcttaatgaggccctagtcgaaccgctgcccgacaaatatgaccgcgagatctactccaactccactggcttttcaaactgcatacagactcaagacccgtacatccaagccttccaacatcagcaagccaaggacgagacattgttctgggcaaccgtcgagaagaggctcgcagcatctacgccgaaggacaactggacagaattcaggactaagagacctctgggtgacgtgctttggctcgcgtacaagcgggcgatggagctcccagatgagcccatcaagtttaacccagagctctggtgggcatgtgcagatgaggtgcaaaagacctacctctccaagcccatacacgcactcaagaacggaattcttcggcagtcacccgactttgactggaacaaactgcagattttcctcaagtcacagtgggttaagaaaattgacaaaatcgggaaaattgacgtcaacgctggacagacaattgccgcattttaccaaccaaccgttatgctgtttggaaccatggcgagatacatgcgccgcatccgcgacacttatcaacccggcgacatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtactgaaagccctgcaccatgatatccctcatgaggttgtggaagcctacgtagccctcaaactcaactcaaaaatgtttctgggcacactggcgattatgagattaactggtgagggacccaccttcgacgctaacacagagtgcaacatcgcttacacacacgcccggttcgagatcccaaagaatgtggcgcaaatgtacgcgggtgacgactgtgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttgctacctacatacgagctaggtgatgcactctacgaaatcttcgacgagaggcaaatgaacgcgcactatcagtcggtcaggacgcttatcacatgcgcccacaccaaagtcctccgagttgcacaggcacttcaggaagactgcaccctctttagctccatctaacaggttttgagttaggctaactccactgacgaattaaataacaatggatagtgaaatagttgaacgactaacaaagcttggtttcgtcaaaacttcacacacgcacatcgctggcgagcccctcgtgatccacgctgttgctggggccggtaaaaccaccctccttcggtccctacttgaattacctggagtggaagtcttcacaggcggggagcacgatcctccaaacttgtcagggaaatatatccgctgtgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgttgacgctttgcgtagggttggtttcgacatcacctttgccggcacgcagactgaagactacggattccaggaaggccatctctacactagtcaattttacggacaggtcatttcacttgacacgcaggcccataagattgctgtgcgccacggacttgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctcgtctttttcatgctcacacgcaacaatctgccacacgttggtgataacatccactcactaccccacggaggcagttacattgacggtaccaagtccatcaactaccgcccacctgcgtcacgctacccctcatctaacttattcgctttcgctccaccaatactcgccgcggtactctttttcctcacacagccatatctagctaccagacgatccaggtgcgttcggtgcttcgttgtccacggggcatgcacgaatcacacctagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccagcatcagtgaagagaaacaacccacctcaagtgtgacctcatcatttcaggacacaagggcaacagtcgacagctatcgatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaatctagacactcaacgaccgcattgagggtgttagggtaaccagcatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacgatcaaacaaggcgcccagtgacgaggagttggtacgcatcatcaacgcggcgcagaagcgaggcctcacacccgcggcctttgttcaagcagctataatcttcaccatggaatccatggacaagggcgccaccgactccacgattttcacgggaaaacacaacactttcccaatgaaaagtctggcgctagcttgcaaagatgctggcgtgcccgtgcacaaactttgctacttctataccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagatcttcaagaaggacaatttgagtcaggcagcatccagaaaccaactcctaggaacgcaagcctccatcacgcgcgggagactcaacggcgcaccagcactaccaaacaatgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaaaaaaaaaaaaaaaaaaaaaaaaaaa

SEQ ID NO: 14 - DNA, 7044 bp, Artificial Sequence
  Sequence of the vector PV139wa.sgp2/101-GFP where the duplicated sgp2 is 101 nt-long. The start codon which is present in the duplicated region was mutated by a T
gaaaactcttccgaaaccgaaactgactgaaactacctcgaccgaccttagaacccaagaacccaacgggtgcggccactatgtctatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggccgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtattggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctccgaaaggcagtatgacccggacggtgaactgatctctcctatcctgagcacccgaaggttgcctaaaactcccataaacacaacactctacgccacgctagacaagattgcacgctgcccaactttctacaagcctgacacagatcgcgcgcagacttacgctcgcgacgtcatggcggggaaaaccggtgccattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggtcaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctgcctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgcgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactcttcttcctcacacagccatatctagccaccagacgatccaggtgcgttcggtgcttcgttgtccacggggcatgcacgaatcacacttagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaagggcaacagtcgacagctatcgatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaatctagacactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctacaccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaa

SEQ ID NO: 15 - DNA, 7091 bp, Artificial Sequence
  Sequence of the vector PV139wa.sgp2/170-GFP (170-nt long sgp2 duplication)
gaaaactcttccgaaaccgaaactgactgaaactacctcgaccgaccttagaacccaagaacccaacgggtgcggccactatgtctatcgaggcagttttcgaccaggttaccgacccatcgctccgcgctgtgattcaggaggaagcgcacaaacagatcaaagatttatttaaggaaacgacgcgctgcaatccctactccataccgcaagctgggcgcaaggttttggaaaagtacgccatcccctacaacccgtactctctcaaactacaccctcacgcagcctcaaaagcgtttgaagtgtcgctctacgaggccgcgtctaactacctcccctccacctcctcaactcctgtcacattcatgttcacaaaaccgggcaagctcagattctttaggcgccgaggtcatgtggacaaattcgttaatgctgacatagttccaagagacttggctagatacccacgcgacacagtctacagttatctgcccgagatcaccaccacacacgctttcattggcgacaccctgcatcacttcggtgaggactttctcgtcgaggttttctccaggtcaccgaaactagaagtgcttctagccaccatggtattaccacccgaagccttttacagaatggagtccctccatccctcggtttacactctcctctacagggacgaccgattcctatacctgcctggtggcctatctggcggtgagtacgaacatcgctataaggacctaaactggctaacatttggcacggttacgcacggcgggatcactatcacaggagaacgcattgagactaaggccgcgaatcatcttttcctcttcagacgagggcgactagcgacaccaaaattccgctcattcgatatgcccgagcctatggtcctgcttcccaaggttttccgccccgcaaagtacaatgtacagaagccaattccccgggagaaagcaaacaaatggttgatgtacgttaaatccatcggcaatgccaccattcgtgacgtatgggctaagctgaggcaaaccatagccaatgcagacattggactcttctcgcccactgagctcgtgcatctcacgaattacttcctgctcctgggccggcttgactcacacaactccttcgaccaagtattggccgacagtgtgctgaaagcatggttcagaccaatggtcgcaaagcttcaggagatcaagcacaaactcatggggcagacccaattcatgcaactctgccaagcgctagagatgacggaggtggacctcgtctttgaggtccgggactccaagaccccccacaaacaagctgtgccattggaccgtgaaattgaaaacgttttgttggaaggagtctcatcggagccaacttacactgaaaccgaaggcgttgctgatggtccacttccccccccaatgcaaactgcagccgagccgtccgcgacctcagacgagcctgagagctctagctcgcgtgaaattgagcaccaaccggcgcctgagatcacgcttgacgaggaagaacctcagcgagacgatctgccttgggacgcttggagaacacaattaagggcgcttggcttcgaggcctccgaaaggcagtatgacccggacggtgaactgatctctcctatcctgagcacccgaaggttgcctaaaactcccataaacacaacactctacgccacgctagacaagattgcacgctgcccaactttctacaagcctgacacagatcgcgcgcagacttacgctcgcgacgtcatggcggggaaaaccggtgccattctcaagcaacaaccctttgagtggaaaaccacgctcaagcgcaagactaaagaggaaccgaaggaaattcaccttgcggtgttgcatggtgcgggcgggtcgggcaaatcctacgcactgcaggaatttatgcggaacaaccctgacacaccaattacggtcatcctgccgactaacgagctcagggccgactggaagaaaaaattgcccgcccacgacaaagacacatttatgacatacgaaaacgcgctcttgtgccctcgtggagacatcttcattatggacgattacacaaaattgcccaggggctacattgaggctttcgtgcagaatgcacctgccctctcacttctgatactcaccggtgaccccaaccaagccgaacactttgagaccactgaggacaatgaaattaacagcctcgcccccgcctcagtggtcttcggcaagttctctaggtaccacataaatgccacacaccgcaaccccagaaacttggcaaacgccctcggtgtttactccgagacgcccggggaggtcaaagtgctttacacgaggaacatcaagaccggttatcacaatctcgtgccctcacaaatgaagatgagaaattacgcctcactcgggcagcgagcgtccacctatgcgggttgtcaggggatcaccgcgccccgcgttcaaatcatcctagactccgacacaccccggtgcaccaggcaagtcatgtacactgcactctcaagggccacgacggaagtggtgctctgcaacacgatgccggatgagaaaagctttttccagaaggttgaagcaacaccgtacctcaaagccatcctcaacctcaacaaagagattaaagtcactgagggtgacttgacagaagaaccgccgagggagcccgctcctcccaccacacacctgcctgttgaaaacagaattattcttaatgaggccctagtcgaaccgctgcccgacaaacatgaccgcgagatctactccaactccactggcttttcaaactgcatacagactcaagacccgtacatccaagccttccaacatcagcaggccaaggacgagacattgttctgggcaaccgtcgagaagaggctcgcagcatctacgccgaaggacaactggacagaattcaagaccaagagacctctgggtgacgtgctttggctcgcgtacaagcgggcgatggagctcccagatgagcccatcaaatttaacccagagctctggtgggcatgtgcagacgaggtgcaaaagacctacctctccaagcccatacacgcactcaagaacggaatccttcggcaatcacccgactttgactggaacaaactgcagattttcctcaagtcacagtgggttaagaagattgacaaaatcgggaaaattgacgtcaacgctggacagacaattgccgccttttaccaaccaaccgttatgctgtttggaaccatggcgagatacatgcgccgcatccgcgacacttatcaaccccgcgaaatactcatcaattgcgagaagaaccagaagcacatttcgaagtgggtcgagagcaattggaaccaccgcctacctgcttacaccaatgacttcactgcttacgaccaaagccaagacggggctatgttacagtttgaggtgctgaaagccctgcaccatgatatccctcatgaggttgtggaagcctacgtagcccttaaactcaactcaaaaatgtttctgggcacactggcgattatgagattaactggtgagggacccaccttcgacgctaacacagagtgcaacattgcttacacacacgcccggttcgagatcccaaagaatgtggcgcaaatgtacgcgggtgacgactgcgcgctcaactgcaggcccgttgaacggcagtccttcctgcctcttgtggagaaattcaccctgaaatcaaaacctaaagtatttgagcaaaaagttgggtcatggcctgagttctgcggcaatctgatcaccccacggggctacctcaaggatcccatgaagctacaacactgcctgcaactggcacagaggaagaaaccatccgaacctgggtcgcttaaagacgttgctgagaactacgctatggacttactacccacatacgagctaggggatgcactctacgaaatcttcgacgagagacaaatgaacgcgcactatcagtcggtcaggacgcttatcacatgcgcccacaccaaagtcctccgagttgcacaggcacttcaggaagactgcaccttctttagctccatctaacaggttttgagttaggctaactccactgacgaattgaataacaatggatagtgaaatagttgaacgactaacaaagcttggtttcgtcaagacttcacacacgcacatcgctggcgagcccctcgtgattcacgccgttgctggggccggtaaaaccaccctccttcggtccttacttgaattacctggagtggaagtcttcacaggcggggagcacgatcctccaaatttgtcagggaaatatatccgctgcgctgcaccccctgtggccggtgcatacaacattcttgacgagtaccccgcgtacccaaattggcgatcgcaaccctggaacgtcctaatcgccgacaacctacaatacaaagaacccacagctcgcgcccactacacatgcaatcgcactcaccgcctggggcagctcactgtcgacgctttgcgtagggttggtttcgacatcacctttgccggcacgcagactgaagactacggattccaggaaggccatctctacactagtcaattctacggacaggtcatttcacttgacacgcaggcccataagatcgctgtgcgccacggactcgcacccctgtccgctttagaaacccgggggctggaatttgatgagaccactgtgataacgactaaaacctcgctggaggaagtgaaggacaggcacatggtctatgtcgctctcacaaggcacaggcgcacctgccatctctacaccgctcactttgcgccctccgcctgacaacacgaaagccatactaactgtagctataggaatagccgcctccctagtctttttcatgctcacacgcaacaatctgccacacgtcggtgataacatccactcactaccccacggaggaagttatattgacggtaccaagtccatcaactaccgcccacctgcgtcacgctacccctcatccaacttactcgctttcgctccaccaatactcgccgcagtactcttcttcctcacacagccatatctagccaccagacgatccaggtgcgttcggtgcttcgttgtccacggggcatgcacgaatcacacttagttgttatattagcgctgttacttatagctctgtggtgtctaagcactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaacactcgacccgttcaaccatcgtgccatgtcgaaatcaacggccactccatcatcgtcaccggaaactgctggcactccactcaacgaccgcattgagggtgttagggtaaccaacatcagtgaagagaaacaacccacctcaagtgtgacctcatcatttcaggacacaatggcaacacaaaatgccgacgtcactgatgcgacggactacaagaaaccgcctgctgaaactgagcagaaggcactcaccattcaaccacggtcaaacaaggcgcccagtgacgaggagttggtacgcatcatcaacgcggcgcagaagcgaggcctcacacccgcggcctttgttcaagcagctatagtcttcaccatggaatccatggacaagggcgccaccgactccacgattttcacgggaaaatacaacactttcccaatgaaaagtctggcgctagcttgcaaagatgctggcgtgcccgtgcacaaactttgctacttctacaccaagccggcttacgcgaaccgtagggtcgccaaccagccgcctgctcgctggactaacgagaatgtgcccaaagctaacaagtgggcggctttcgacaccttcgacgcacttctcgacccatacgtagtcccatcctctgtaccgtacgatgagcccacgccagaggatcgccaagtcaatgagattttcaagaaggacaatttgagtcaggcagcatccagaaaccaactcctaggaacgcaagcctccatcacgcgcgggagactcaacggcgcaccagcactaccaaacaacgggcagtacttcatcgaggcacctcagtgatcagtagtatgataccaataaataaatcgggcgaatccgcgcctcctgactatgggcaggtttacggaccaagctgtatcgagatacgacctaacagtaacgcagctaaggggtgaatgcacacatcgcttataaaaaaaaaaaaaaaaaaaaaaaaaaaaaaaaaaaaaaaa

SEQ ID NO: 16 - DNA, 18 bp, Artificial Sequence
  5′35Sp
ctcgcatgcctgcaggtc

SEQ ID NO: 17 - DNA, 38 bp, Artificial Sequence
  5′FoMV-3′35Sp
cggtttcggaagagttttccctctccaaatgaaatgaa

SEQ ID NO: 18 - DNA, 38 bp, Artificial Sequence
  3′35Sp-5′FoMV
ttcatttcatttggagagggaaaactcttccgaaaccg

SEQ ID NO: 19 - DNA, 28 bp, Artificial Sequence
  SpeI-FoMV1040R
tatcactagtggtttgcctcagcttagc

SEQ ID NO: 20 - DNA, 30 bp, Artificial Sequence
  pBxs-fw
atactcgaggcgtggatcccgctgggcctc

SEQ ID NO: 21 - DNA, 30 bp, Artificial Sequence
  pBxs-rev
gttatactagttttttccttatgcggcctt

SEQ ID NO: 22 - DNA, 38 bp, Artificial Sequence
  pB-Fmcs-10-fw1
cagttaacttactctagacactcaacgaccgcattgag

SEQ ID NO: 23 - DNA, 36 bp, Artificial Sequence
  pB-Fmcs-10-rev1
gtcgacttgttcggccgagtgccagcagtttccggt

SEQ ID NO: 24 - DNA, 35 bp, Artificial Sequence
  pB-Fmcs-10-fw2
agttggcgcgccaatcagttaacttactctagaca

SEQ ID NO: 25 - DNA, 29 bp, Artificial Sequence
  pB-Fmcs-10-rev2
atcgatagctgtcgacttgttcggccgag

SEQ ID NO: 26 - DNA, 37 bp, Artificial Sequence
  pB-Fmcs-fw1
cagttaacttactctagacgcattgagggtgttaggg

SEQ ID NO: 27 - DNA, 36 bp, Artificial Sequence
  pB-Fmcs-rev1
gtcgacttgttcggccgtcgttgagtggagtgccag

SEQ ID NO: 28 - DNA, 35 bp, Artificial Sequence
  pB-Fmcs-fw2
agttggcgcgccaatcagttaacttactctagacg

SEQ ID NO: 29 - DNA, 29 bp, Artificial Sequence
  pB-Fmcs-rev2
atcgatagctgtcgacttgttcggccgtc

SEQ ID NO: 30 - DNA, 31 bp, Artificial Sequence
  GFP5′-ClaI-fw
aggtcaatcgatatggtgagcaagggcgagg

SEQ ID NO: 31 - DNA, 34 bp, Artificial Sequence
  GFP3′-XbaI-rev
tatgcttctagattacttgtacagctcgtccatg

(19) United States
(12) Patent Application Publication
(10) Pub. No.: US 2020/0270621 A1
(43) Pub. Date: Aug. 27, 2020

(54) VECTORS AND METHODS FOR GENE EXPRESSION IN MONOCOTS

(71) Applicant: Rothamsted Research Limited, Harpenden Herts (GB)

(72) Inventors: Konstantin Kanyuka, Harpenden Herts (GB); Kim E. Hammond-Kosack, Harpenden Herts (GB); Clement Bouton, Harpenden Herts (GB)

(21) Appl. No.: 16/338,156

(22) PCT Filed: Oct. 6, 2017
(86) PCT No.: PCT/GB2017/053029

§ 371 Date: Mar. 29, 2019

(30) Foreign Application Priority Data
Oct. 7, 2016 (GB) 1617109.2

(51) Int. Cl. C12N 15/82; C12N 7/00

(52) U.S. Cl. CPC C12N 15/8203; C12N 7/00; C12N 2770/00043; C12N 15/8241; C12N 15/8205

(57) ABSTRACT

The invention relates to the field of genetic engineering tools for gene expression in plants. Specifically, the invention concerns modified Foxtail Mosaic Virus (FoMV) vectors comprising polynucleotide sequences which are capable of driving expression of a gene of interest in a plant host. Accordingly, the invention concerns FoMV-based expression vectors comprising said polynucleotides, compositions comprising modified FoMV vectors, methods of generating gene expression in plants infected with the modified FoMV vectors. The expression vectors, compositions, plants and methods of the present invention find application in many fields of biotechnology, including, for example, gene characterization, protein production and agricultural biotechnology.

The invention relates to a modified Foxtail Mosaic Virus (FoMV) vector for the virus-mediated expression of proteins in plants and methods of expressing protein in plants using modified FoMVs of the invention.

### BACKGROUND

The ability to express proteins of interest in plants is an important tool in the discovery of function and characterization of both native and heterologous proteins. Plants are also an attractive system for heterologous protein expression in particular, because of their potential for scalability, low production and maintenance costs, reduced requirements for investment in infrastructure, potential for using lower value land in production and in the case of therapeutic proteins, fewer problems associated with post-translational modifications, processing and contamination of the protein fraction that are often associated with prokaryotic expression systems. However, the potential of plants as expression systems for proteins of interest has not yet been fully realized, primarily a consequence of lower level of transgene expression achieved in plant systems and an inability to produce protein in a wide range of plant hosts, as may be desired. This is particularly the case in monocot systems, which as many major crop species are monocots, arguably have the greatest potential to produce proteins of interest on a large scale.

Plant viruses have been widely used as vectors for both heterologous gene expression and virus-induced gene silencing (VIGS) in plants. Viral vectors capable of expressing heterologous proteins in plants provide valuable biotechnological tools to complement genetic and transgenic technologies. Plant viral vectors enable proteins to be overexpressed in different genetic backgrounds. Additionally, the efficiency with which recombinant protein can be obtained using plant viral vectors has resulted in their widespread use as tools for protein expression in plants. Because of these inherent advantages, plant viral vectors have been developed and used in both dicot and monocot plants.

Chapman et al., originally described the development of an expression vector from Potato virus X (PVX) (Chapman et al., 1992, The Plant Journal 2:549-557) for use in dicots. One of the strategies described in this paper was the expression of the gene of interest (GOI) via duplication of the virus coat protein sub-genomic promoter (CP-sgp). The successful (transcription) expression of GUS, a 1.9 kbp-long gene, is reported in inoculated and systemic leaves. However, the gene of interest (GOI) was partially unstable and thus could be lost in the upper non-inoculated leaves or during subsequent virus passaging onto healthy plants.

Barley Stripe Mosaic Virus (BSMV) is the most commonly used VOX (virus-mediated overexpression) vector in monocots, such as wheat. Wheat Streak Mosaic Virus (WSMV) has also been used for protein expression in wheat. However, these currently available vectors have some important limitations. Specifically, both existing BSMV and WSMV vectors only allow the expression of heterologous proteins as fusions to the viral proteins and, in the case of BSMV, allow expression of only relatively small proteins (those <200 amino acids in length). A further issue with the BSMV vector is that it induces pronounced, sometimes severe symptoms, which can hinder the phenotypic assessment of host plants. In particular, the symptoms can obscure the effects of protein expression in the plant background, and or render the infected plants unusable for their desired application which limits their application as a biotechnological tool.

For example, the use of Barley stripe mosaic virus (BSMV) to express a gene of interest (GOI), the fungal effector ToxA—in bread wheat (Triticum aestivum), has been described (Manning et al., 2010, New Phytologist 187:1034-1047). However, in the system described in this paper, ToxA coding sequence was fused to the 5′ terminus of the virus gene γb. Consequently, the heterologous protein expressed using this vector is subject to fusion-related co-translational processing such as cleavages which may interfere with its localisation and/or intrinsic activity.

Similarly, Choi et al. (Choi et al., 2000, The Plant Journal 23:547-555) described a virus vector based on Wheat streak mosaic virus (WSMV). However, this virus expresses all its proteins from a polyprotein precursor. Thus, the gene of interest (GOI) has to be expressed and processed from a longer polyprotein and therefore suffers from the same fusion-related limitations.

Recently, Foxtail Mosaic Virus (FoMV) has been developed as a vector for virus-induced gene silencing (VIGS) in monocots, including wheat, barley and foxtail-millet. The sequence of the gene to silence was inserted between duplicated CP-sgp (Liu et al. 2016, Plant Physiology 171:1801-1807).

Attempts have been made, with very limited success, to develop FoMV as a vector for protein expression in monocots. For example, Liu and Kearney described a number of different FoMV expression vectors for protein expression in monocots (Liu and Kearney, 2010, BMC Biotechnology 10:88). In this system the GOI was placed under the control of the sub-genomic promoter 1 (sgp1). The binary FoMV-vector was delivered to plants by agro-infiltration. However, all of the FoMV expression vectors described were deficient in systemic movement, i.e. unable to move systemically in the infected plants. Additionally, co-infiltration of a vector encoding the suppressor of gene silencing p19 was required to achieve high expression of the GOI.

Some limitations of known virus-mediated overexpression (VOX) systems for wheat and maize are that these vectors do not allow expression of native proteins, or proteins of greater than 150 amino-acids, and they are characteristically low throughput.

Functional analysis of genes encoding small secreted effector proteins predicted in the genomes of wheat-infecting fungal pathogens currently relies on labour-intensive methods when assessing their function in wheat. Typical experiments involve expression of candidate effectors in heterologous in vitro systems (such as the yeast Pichia pastoris or E. coli) followed by syringe infiltration of purified effectors into wheat leaves and analysis of resulting induced phenotypes/responses. More efficient ways of performing functional analysis of fungal effector proteins are needed.

### BRIEF SUMMARY OF THE DISCLOSURE

The invention relates to the development of the Foxtail Mosaic Virus (FoMV) as a vector for the virus-mediated expression of proteins in plants. The invention is based on a discovery that duplication of certain nucleotide sequence elements of one of the Foxtail Mosaic Virus (FoMV) viral promoters in a vector enables the transient expression of polypeptides in plants. By harnessing specific viral promoter sequence elements in this way, the present invention for the first time makes it feasible to readily express a protein of interest in plants, such as wheat, without the associated size and processing limitations imposed by the currently available panel of vectors. The modified FoMV vectors, methods and compositions described herein can therefore be used to express native or heterologous proteins in a plant background.

In seeking to overcome the practical disadvantages associated with the existing vector systems, the inventors provide a modified Foxtail Mosaic Virus (FoMV) viral vector for application as a tool for achieving transient protein expression in plants. The modified FoMV of the invention has been found to have uniquely advantageous mechanistic features which enable expression of proteins larger than 200 amino acids.

The newly developed FoMV vector overcomes the limitations of the currently available VOX vectors developed for wheat. Unlike the BSMV- and WSMV-VOX vectors, the modified FoMV vector allows the expression of native heterologous proteins. The gene of interest can thus be cloned without being fused to a viral gene. Consequently, the heterologous protein will avoid fusion-related co-translational processing such as cleavages which may interfere with its localisation and/or intrinsic activity. Moreover, polypeptides longer than 200 amino acids, which is the limit size of the heterologous proteins expressed from the currently available BSMV-VOX vectors, can be stably expressed with the modified FoMV vectors disclosed herein.

The modified FoMV vectors described herein overcome several significant limitations of vectors based on Barley Stripe Mosaic Virus (BSMV) and/or Wheat Streak Mosaic Virus (WSMV) for protein expression available to date and therefore are of greater general utility. Firstly, FoMV induces only moderate symptoms on wheat. This lack of strong symptoms is a very useful feature when determining plant phenotypes induced by heterologous proteins, especially those predicted to induce or regulate plant defense, cell death and/or senescence pathways.

Secondly, FoMV has a broad range of hosts including various monocots and dicots.

Thirdly, FoMV has a monopartite genome and does not express its genes from a polyprotein and therefore the cloned heterologous genes do not need to undergo any co-translational processing which may interfere with their localisation and/or activity.

Fourthly, FoMV described herein can express proteins which exceed 200 amino acids in length and also which exceed 600 amino acids in length, and therefore overcome a significant limitation of vectors available to date.

Additionally, FoMV can spread systemically and therefore can infect whole plants or plant parts. Finally, presence of the suppressor of gene silencing p19 is not required to achieve (high levels of) expression of the protein of interest.

Accordingly, the present invention provides a modified Foxtail Mosaic Virus (FoMV) for use in the expression of a gene of interest (GOI) in a plant, plant part or plant cell.

The present invention also provides a modified FoMV for expression of a gene of interest (GOI) in a plant, plant part or plant cell, wherein the virus comprises a first sgp2 promoter and at least a second sgp2 promoter; and wherein the GOI is under transcriptional control of the at least second sgp2 promoter.

The GOI preferably encodes a protein or polypeptide which may be a native or an heterologous protein or polypeptide to the plant in question. (The term “protein” and “polypeptide” are used interchangeably herein). In some cases the polypeptide may be of microbial or animal/human origin.

Part of the advantage of the modified FoMV of the invention is the ability to transiently express polypeptides in plants which are relatively large, e.g. more than 200 amino acids in length. In particular, the inventors have demonstrated that modified FoMV of the invention can be used to express even larger proteins, e.g. of 600 amino acids or more. Therefore, polypeptides suitable for expression using the FoMV of the invention may be at least 400, 500 or 600 amino acids long; and/or they may be not more than about 800, 900 or 1000 amino acids in length.

In other aspects, the GOI may be a protein non-coding sequence. Examples of these include polynucleotide sequences which encode artificial microRNA. An effect of the expression of miRNA will be to downregulate host plant gene expression. Other examples of protein non-coding polynucleotide sequences include long noncoding RNAs which would function as miRNA decoys/target mimics/sponges for downregulating plant miRNA and hence indirectly upregulating/overexpressing target plant genes and their encoded proteins.

In accordance with the invention, the at least second sgp2 promoter is preferably located 5′- of the native (i.e. first) sgp2 promoter. It is not an essential requirement of the invention that the at least second sgp2 promoter has the same polynucleotide sequence as the first sgp2 promoter. The first and the at least second sgp2 promoter sequences may be identical. Conveniently, the first and the at least second sgp2 promoter sequences may be different. Preferably, the polynucleotide sequence encoding the protein of interest is under operable control of the at least second sgp2 promoter. Preferably, the at least second sgp2 promoter drives expression of the polynucleotide sequence encoding the protein of interest in the infected (i.e. host or recipient) plant. Preferably, the polynucleotide sequence encoding the protein of interest is under transcriptional control of the at least second sgp2 promoter whereas the first (3′) sgp2 promoter controls expression of the virus coat protein (CP).

Therefore in certain embodiments, the first sgp2 promoter is 5′ of a polynucleotide encoding the virus coat protein (CP) and the at least second sgp2 promoter and polynucleotide encoding the polypeptide of interest are 5′ of the first sgp2 promoter.

By “modified FoMV” herein is meant not only viral particles comprising coat protein and RNA, but also the DNA viral vector form, and the RNA viral vector form, both of which can be used to infect plants. A mixture of any two or three of these forms is possible. In some embodiments, the infective material provided for use is already infected plant material which is simply homogenised or ground and used directly to infect fresh plant material. Included in the invention therefore is genomic RNA (gRNA) of modified FoMV as herein described.

A modified FoMV of the invention may comprise two or more sgp2 promoters. In further aspects of the invention it is envisaged that modified FoMVs of the invention may comprise three or more, four or more, five or more, six or more, seven or more, eight or more, nine or more or ten or more sgp2 promoters if the application requires it. Each of these promoters may independently control expression of a protein of interest, which may be multiple copies of the same coding sequence, or alternatively, may be coding sequences which encode different proteins of interest, for simultaneous expression in a host plant. Furthermore, as the application requires, the at least second sgp2 promoter may further comprise additional elements which influence expression of the protein of interest in the host plant.

The term “under transcriptional control” is well known in the art. mRNA transcripts encoding the polypeptide of interest are produced in plant cells infected with modified FoMVs described herein by placing the encoding polynucleotide under the control of the at least second sgp2 promoter sequence. Accordingly, it will be understood that the at least sgp2 promoter regulates expression at the mRNA transcript level of the polynucleotide encoding the polypeptide of interest. In preferred embodiments, the polynucleotide encoding the polypeptide of interest is placed directly downstream of the at least second sgp2 promoter in order that it drives production of the transcript encoding the protein of interest in the infected plant, plant part or plant cell. Ideally, high-levels of transcript accumulation are generated in infected plant cells. Ideally, the at least second sgp2 promoter drives high-levels of polypeptide expression in infected plant cells.

The present invention also provides a modified FoMV, wherein the at least second sgp2 promoter comprises a polynucleotide sequence of SEQ ID NO: 1 or a sequence of at least 90% identity thereto.

In preferred aspects, the at least second sgp2 promoter comprises a nucleotide sequence of at least 95%; preferably at least 96%; more preferably at least 97%; even more preferably at least 98%; still more preferably at least 99% of SEQ ID NO: 1. Optionally, the at least second sgp2 promoter comprises a polynucleotide sequence of SEQ ID NO: 1 which includes one or two contiguous or non-contiguous polynucleotide substitutions, deletions or insertions with respect to the reference sequence.

Optionally, the at least second sgp2 promoter may comprise a polynucleotide sequence of SEQ ID NO: 2 or a sequence of at least 90% identity thereto. Preferably, the at least second sgp2 promoter comprises a nucleotide sequence of at least 95%; preferably at least 96%; more preferably at least 97%; even more preferably at least 98%; still more preferably at least 99% of SEQ ID NO: 2. Optionally, the at least second sgp2 promoter comprises a polynucleotide sequence of SEQ ID NO: 2 which includes one, two, three, four, five or six contiguous or non-contiguous polynucleotide substitutions, deletions or insertions.

Optionally, the at least second sgp2 promoter may comprise a polynucleotide sequence of SEQ ID NO: 3 or a sequence of at least 90% identity thereto. The at least second sgp2 promoter may optionally comprise a nucleotide sequence of at least 95%; preferably at least 96%; more preferably at least 97%; even more preferably at least 98%; still more preferably at least 99% of SEQ ID NO: 3. Optionally, the at least second sgp2 promoter comprises a polynucleotide sequence of SEQ ID NO: 3 which includes one, two, three, four, five or six contiguous or non-contiguous polynucleotide substitutions, deletions or insertions with respect to the reference sequence.

Optionally, the polynucleotide sequence of the at least second sgp2 promoter may further comprise a polynucleotide sequence of SEQ ID NO: 4. Preferably, the at least second sgp2 promoter comprises a nucleotide sequence of at least 98%; preferably at least 99% of SEQ ID NO: 4. Optionally, the at least second sgp2 promoter comprises a polynucleotide sequence of SEQ ID NO: 4 which includes one or two contiguous or non-contiguous polynucleotide substitutions, deletions or insertions with respect to the reference sequence.

Preferably, the polynucleotide sequence of the at least second sgp2 promoter may comprise; a polynucleotide sequence of SEQ ID NO: 5 or a sequence of at least 90% identity therewith; or a polynucleotide sequence of SEQ ID NO: 6 or a sequence of at least 90% identity therewith; or a polynucleotide sequence of SEQ ID NO: 7 or a sequence of at least 90% identity thereto. More preferably, the polynucleotide sequence of the at least second sgp2 promoter comprises a polynucleotide sequence of SEQ ID NO: 5, SEQ ID NO: 6 or SEQ ID NO: 7, or a sequence of at least 95% identity therewith; preferably at least 96%; more preferably at least 97%; even more preferably at least 98%; still more preferably at least 99% of either SEQ ID NO: 5, SEQ ID NO: 6 or SEQ ID NO: 7. Optionally, the at least second sgp2 promoter comprises a polynucleotide sequence of SEQ ID NO: 5, SEQ ID NO: 6 or SEQ ID NO: 7 which includes one, two, three, four, five or six contiguous or non-contiguous polynucleotide substitutions, deletions or insertions with respect to the reference sequence.

The duplication of the sgp2 elements disclosed herein may suitably be integrated into different vector isolates, that of pCF [SEQ ID NO: 8] or PV139wa [SEQ ID NO: 9], in order to drive expression of a protein of interest in a recipient plant.

The invention also provides a Foxtail Mosaic Virus (FoMV) DNA expression construct comprising from 5′ to 3′ polynucleotide sequences encoding a strong heterologous promoter that is active in plants, followed by the viral ORF1, sgp1 promoter, ORF2, ORF3, ORF 4, at least two sgp2 promoters, coat protein (CP) and nopaline synthase terminator (nos), wherein the ORF2 overlaps with ORF3 and ORF3 overlaps with ORF4, and ORF4 includes the start codon of ORF5A, wherein the polynucleotide sequence comprised in or between the duplicated sgp2 promoters includes insertion site(s) for a gene of interest (GOI).

In such FoMV expression constructs, the duplicated portion of the sgp2 promoter may have a polynucleotide sequence comprising SEQ ID NO: 2 or a sequence of at least 90% identity thereto; or SEQ ID NO: 3 or a sequence of at least 90% identity thereto; or SEQ ID NO: 4; or SEQ ID NO: 5 or a sequence of at least 90% identity thereto; or SEQ ID NO: 6 or a sequence of at least 90% identity thereto.

In examples of certain FoMV expression constructs the insertion site may comprise a series of restriction sites; preferably SalI-ClaI-AscI-HpaI-XbaI or NotI-ClaI-AscI-HpaI-XbaI.

In other examples of FoMV expression constructs of the invention, the convenience and speed of insertion of a GOI is improved by incorporation of att recombination cloning sites; preferably attB or attR. In a particularly convenient embodiment, expression constructs have a Gateway® cassette inserted between the sgp2 promoters. The engineering and operation of a Gateway® cassette (ThermoFisher Scientific) in the context of the invention will be familiar to a person of average skill in the art and with reference to the manufacturers instructions and product literature.

Accordingly, the invention provides a modified FoMV, wherein the virus comprises the sequence SEQ ID NO: 8 or a sequence of at least 80% identity therewith, and wherein the modified FoMV further comprises a polynucleotide sequence of SEQ ID NO: 1 or SEQ ID NO: 2 or SEQ ID NO: 3 or SEQ ID NO: 4 or SEQ ID NO: 5 or SEQ ID NO: 6 or SEQ ID NO: 7. Preferably, the modified FoMV comprises the sequence SEQ ID NO: 8 and additionally comprises a polynucleotide sequence of SEQ ID NO: 2. More preferably, the modified FoMV comprises the sequence SEQ ID NO: 8 and additionally comprises a polynucleotide sequence of SEQ ID NO: 2 and SEQ ID NO:4. Even more preferably, the modified FoMV comprises the sequence SEQ ID NO: 8 and additionally comprises a polynucleotide sequence of SEQ ID NO: 5. Still more preferably, the modified FoMV comprises the sequence SEQ ID NO: 8 and additionally comprises a polynucleotide sequence of SEQ ID NO: 6. Alternatively, the modified FoMV comprises the sequence SEQ ID NO: 8 and additionally comprises a polynucleotide sequence of SEQ ID NO: 7.

In particular, the invention provides a modified FoMV having a nucleotide sequence of SEQ ID NO: 10, i.e. modified pCF vector comprising the 45 nt sgp2 promoter duplication.

Alternatively, the invention provides a modified FoMV having a nucleotide sequence of SEQ ID NO: 11, i.e. modified pCF vector comprising the 55 nt sgp2 promoter duplication.

Alternatively, the invention provides a modified FoMV having a nucleotide sequence of SEQ ID NO: 12, i.e. modified pCF vector comprising the 90 nt sgp2 promoter duplication.

Alternatively, the invention provides a modified FoMV having a nucleotide sequence of SEQ ID NO: 13, i.e. modified pCF vector comprising the 101 nt sgp2 promoter duplication.

The above modifications can permit a skilled person to select in a predetermined manner a desired expression pattern in a host plant of interest depending on the application concerned. For example a systemic or localised expression pattern.

Accordingly, the invention also provides a modified FoMV, wherein the virus comprises the sequence SEQ ID NO: 9 or a sequence of at least 80% identity therewith, and wherein the modified FoMV further comprises a polynucleotide sequence of SEQ ID NO: 1 or SEQ ID NO: 2 or SEQ ID NO: 3 or SEQ ID NO: 4 or SEQ ID NO: 5 or SEQ ID NO: 6 or SEQ ID NO: 7. Preferably, the modified FoMV comprises the sequence SEQ ID NO: 9 and additionally comprises a polynucleotide sequence of SEQ ID NO: 2. More preferably, the modified FoMV comprises the sequence SEQ ID NO: 9 and additionally comprises a polynucleotide sequence of SEQ ID NO: 2 and SEQ ID NO:4. Even more preferably, the modified FoMV comprises the sequence SEQ ID NO: 9 and additionally comprises a polynucleotide sequence of SEQ ID NO: 5. Still more preferably, the modified FoMV comprises the sequence SEQ ID NO: 9 and additionally comprises a polynucleotide sequence of SEQ ID NO: 6. Alternatively, the modified FoMV comprises the sequence SEQ ID NO: 9 and additionally comprises a polynucleotide sequence of SEQ ID NO: 7.

Alternatively, the invention provides a modified FoMV having a nucleotide sequence of SEQ ID NO: 14, i.e. modified PV139wa vector comprising the 101 nt sgp2 promoter duplication.

Alternatively, the invention provides a modified FoMV having a nucleotide sequence of SEQ ID NO: 15, i.e. modified PV139wa vector comprising the 170 nt sgp2 promoter duplication. Advantageously, the use of the 170 nt sgp2 promoter duplication in the PV139wa FoMV vector isolate background results in a faster movement of the FoMV through the recipient plant. Therefore where speed and extent of protein expression is desired, the invention may preferably provide a modified FoMV having a nucleotide sequence of SEQ ID NO: 15 or variants thereof.

The above modifications can permit a skilled person to select in a predetermined manner a desired expression pattern in a host plant of interest depending on the application concerned.

The invention provides modified FoMVs having the nucleotide sequence of SEQ ID NO: 8 or SEQ ID NO: 9, but it will be appreciated that these reference sequences include any variant sequence having the defined percentage identity therewith. Such percentage identities include any of the following: a reference nucleic acid sequence and sequences of at least a certain percentage identity are disclosed, e.g. at least 80%, then optionally the percentage identity may be different. For example: a percentage identity which is selected from one of the following: at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8%. Such sequence identity with a nucleic acid sequence is a function of the number of identical positions shared by the sequences in a selected comparison window, taking into account the number of gaps, and the length of each gap, which need to be introduced for optimal alignment of the two sequences.

One further additional feature of the modified FoMV vectors of SEQ ID NO: 9 and variants thereof is that they are derived from a FoMV that has been adapted to wheat through a series of repeated mechanical passages from infected onto initially healthy wheat plants.

There are two advantages associated with modified FoMV vectors based on PV139wa (SEQ ID NO: 9 and variants thereof). Firstly, the efficiency of wheat inoculation with this adapted FoMV is very close to 100%, substantially improving the efficiency of transient expression in recipient plants, when wheat is the target species for protein expression. Secondly, the inventors have found that when the sgp2 promoter elements described herein (i.e. SEQ ID NO: 1, SEQ ID NO: 2, SEQ ID NO: 3, SEQ ID NO: 4, SEQ ID NO: 5, SEQ ID NO: 6, SEQ ID NO: 7 and variants thereof) are duplicated in the PV139wa vector background (SEQ ID NO: 9), the viral vector vectors only induce mild or faint symptoms on infected wheat plants which allows phenotypes relating to protein expression in the host plant to be advantageously divorced from the symptoms of the viral infection. This overcomes a major limitation of the previously available vectors which are available for protein expression in wheat.

Throughout, the invention provides modified FoMVs having reference polynucleotide sequences, but it will be appreciated that these reference sequences include any variant sequence having the defined percentage identity therewith. Such percentage identities include any of the following: a reference nucleic acid sequence and sequences of at least a certain percentage identity are disclosed, e.g. at least 80%, then optionally the percentage identity may be different. For example: a percentage identity which is selected from one of the following: at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8%. Such sequence identity with a nucleic acid sequence is a function of the number of identical positions shared by the sequences in a selected comparison window, taking into account the number of gaps, and the length of each gap, which need to be introduced for optimal alignment of the two sequences.

The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet.

Additionally, the present invention provides a modified Foxtail Mosaic Virus (FoMV) for use in the expression of a polypeptide in a plant, plant part or plant cell, wherein the plant is a monocotyledonous plant, or the plant part or plant cell is of a monocotyledonous plant. Normally, the modified FoMV will be used to drive expression of a polypeptide of interest in a plant, plant part or plant cell selected from Triticum sp., a synthetic hybrid wheat species, Aegilops sp., Hordeum sp., Oryza sp., Zea sp., Avena sp., Poa sp., Secale sp., Sorghum sp., Setaria sp., Panicum sp., Brachypodium sp., Agropyron sp., Lolium sp., Festuca sp., Agrostis sp., Miscanthus sp., Bromopsis sp., Buchloe sp., Bouteloua sp., or Triticosecale sp. Typically, the plant, plant part or plant cell is selected from the group consisting of Triticum sp. and Zea sp.

Optionally, the modified FoMV will be used to drive expression of a polypeptide of interest in a plant, plant part or plant cell selected from Setaria viridis or Setaria italica.

Preferably, the modified FoMV will be used to drive expression of a polypeptide of interest in a plant, plant part or plant cell selected from Triticum aestivum, Triticum durum, Triticum urartu, Triticum monococcum, Triticum boeoticum, Triticum turgidum, Triticum dicoccon, Triticum timopheevi, Triticum polonicum, Triticum turanicum, Triticum spelta, Triticum compactum, Triticum sphaerococcum, or Triticum carthlicum. Most preferably, the modified FoMV will be used to drive expression of a polypeptide of interest in a plant, plant part or plant cell of Triticum aestivum.

Alternatively, the present invention provides a modified Foxtail Mosaic Virus (FoMV) for use in the expression of a polypeptide in a plant, plant part or plant cell, wherein the plant is a dicotyledonous plant, or the plant part or plant cell is of a dicotyledonous plant. Where dicotyledonous plants are the target host for expression of a polypeptide of interest, the modified FoMV will usually be used to drive expression of a polypeptide of interest in a plant, plant part or plant cell selected from Nicotiana sp., and Arabidopsis sp. Preferably the modified FoMV will usually be used to drive expression of a polypeptide of interest in a Nicotiana benthamiana plant, plant part or plant cell.

The invention also provides FoMV viral genomic RNA (gRNA) encoded by an FoMV expression construct as described herein.

The polypeptide or protein of interest, desired for expression in the infected plant may be a native protein to the infected plant or alternatively it may be a heterologous protein. Preferably the invention provides a modified FoMV as described herein, for use in the expression of a polypeptide in a plant, wherein the polypeptide is a heterologous polypeptide. Advantageously, a modified FoMV as described herein is provided, wherein the polypeptide is greater than 200 amino acids in length. Optionally, the polypeptide may be greater than 250 amino acids in length, greater than 300 amino acids in length, greater than 350 amino acids in length, greater than 400 amino acids in length, greater than 450 amino acids in length, greater than 500 amino acids in length greater than 550 amino acids in length, greater than 600 amino acids in length. Optionally, the polypeptide may be in the range 200 to 300 amino acids in length, 300 to 400 amino acids in length, 400 to 500 amino acids in length or 500 to 600 amino acids in length.

The invention includes any host plant cell transformed with a modified FoMV as described herein.

The plant in which a modified FoMV comprising a polynucleotide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 is expressed, may either be a monocotyledonous plant or a dicotyledonous plant, or the plant part or plant cell may be of a monocotyledonous plant or of a dicotyledonous plant. Preferably the plant is a monocotyledonous plant, or the plant part or plant cell is of a monocotyledonous plant.

Commonly, the plant, plant part or plant cell is selected from Triticum sp., a synthetic hybrid wheat species, Aegilops sp., Hordeum sp., Oryza sp., Zea sp., Avena sp., Poa sp., Secale sp., Sorghum sp., Setaria sp., Panicum sp., Brachypodium sp., Agropyron sp., Lolium sp., Festuca sp., Agrostis sp., Miscanthus sp., Bromopsis sp., Buchloe sp., Bouteloua sp., or Triticosecale sp.

Optionally, the plant, plant part or plant cell may be selected from Setaria viridis or Setaria italica.

Preferably, the plant, plant part or plant cell may be selected from Triticum aestivum, Triticum durum, Triticum urartu, Triticum monococcum, Triticum boeoticum, Triticum turgidum, Triticum dicoccon, Triticum timopheevi, Triticum polonicum, Triticum turanicum, Triticum spelta, Triticum compactum, Triticum sphaerococcum, or Triticum carthlicum.

Alternatively, the plant, plant part or plant cell may be selected from Aegilops tauschii, Aegilops bicornis, Aegilops biuncialis, Aegilops columnaris, Aegilops crassa, Aegilops cylindrica, Aegilops geniculata, Aegilops juvenalis, Aegilops mutica, Aegilops neglecta, Aegilops sharonensis, Aegilops speltoides, Aegilops triuncialis, Aegilops umbellulata, or Aegilops ventricosa.

According to the present invention, whole plants, plant parts or plant cells may be infected with a modified FoMV as described herein and capable of transiently expressing a polypeptide or protein of interest. Plant cells which are capable of transiently expressing a polypeptide of interest refer to cells which contain heterologous DNA or RNA, and are capable of expressing the trait conferred by the heterologous genetic material incorporated in the modified FoMV with which it is infected, without having fully incorporated that genetic material into the cell's DNA. Heterologous genetic material encoding the polypeptide or protein of interest may be incorporated into pCF or PV139wa vector isolates as required to suit the application of the invention. Recipient plants, plant parts or plant cells may be infected with more than one FoMV carrying multiple copies of the same, or different polynucleotides which encode a protein(s) of interest. If desired, more than one coding sequence may be incorporated into the modified FoMV, to drive expression of more than one protein in the host plant.

Subsequently, in various applications of the invention, it may be desirable to determine the expression levels of the protein in recipient plants, plant parts, plant tissues or plant cells. Expression levels may be determined by various techniques which are routinely used in the art. In some instances, it may be possible to directly determine functional expression, e.g. as with GFP or by enzymatic action of the protein of interest (P01) to generate a detectable optical signal. However, in some instances it may be chosen to determine physical expression, e.g. by antibody probing, and rely on separate test to verify that physical expression is accompanied by the required function.

In preferred embodiments of the invention, expression of polypeptides of interest will be detectable by a high-throughput screening method, for example, relying on detection of an optical signal. For this purpose, it may be necessary for the protein of interest (P01) to incorporate a tag, or be labelled with a removable tag, which permits detection of expression. Such a tag may be, for example, a fluorescence reporter molecule translationally-fused to the POI, e.g. Green Fluorescent Protein (GFP), Yellow Fluorescent Protein (YFP), Red Fluorescent Protein (RFP), Cyan Fluorescent Protein (CFP), mCherry Protein, Cerulean Protein or Venus Protein. Such a tag may provide a suitable marker for visualisation of functional polypeptide expression since its expression can be simply and directly assayed by fluorescence measurement in recipient plants or plants of interest. It may be an enzyme which can be used to generate an optical signal. Tags used for detection of expression may also be antigen peptide tags. Tags employed for detection of protein expression may optionally be cleavable from the protein if the application requires it. Other kinds of label may be used to mark the nucleic acid including organic dye molecules, radiolabels and spin labels which may be small molecules.

The invention also provides a method of expressing a protein in a plant, plant part or plant cell, comprising infecting the plant, plant part or a plant cell with a modified FoMV as described herein.

That is, the invention provides a method of expressing a protein in a plant, plant part or plant cell, comprising infecting the plant, plant part or a plant cell with a modified FoMV using the PCF vector isolate background or the PV139wa background and further comprising the 45 nt, 55 nt, 90 nt, 101 nt, or alternatively the 170 nt sgp2 promoter duplication in order to drive expression of a polypeptide of interest in a recipient plant.

Optionally, therefore the method comprises infecting the plant, plant part or a plant cell with a modified FoMV of SEQ ID NO: 1, SEQ ID NO: 2, SEQ ID NO: 3, SEQ ID NO: 4; SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or variants thereof. Preferably, the method comprises infecting the plant, plant part or a plant cell with a modified FoMV of SEQ ID NO: 10, SEQ ID NO: 11, SEQ ID NO: 12, SEQ ID NO: 13, SEQ ID NO: 14 or SEQ ID NO: 15. Most preferably, the method comprises infecting the plant, plant part or a plant cell with a modified FoMV of SEQ ID NO: 13.

Modified FoMV Vector Delivery

The FoMV vectors can be delivered in several different ways into the recipient plant, such as for example, wheat.

The vectors may be delivered into the recipient plant by rub-inoculation of plant leaves with the modified FoMV virus particles, with the viral vector DNA or viral vector RNA. In an alternative, the vectors may be delivered into the recipient plant by bombardment of plant leaves with gold particles coated with DNA, RNA or plasmids.

Infection of the plants in accordance with the methods of the invention may involve a single application of the vector to the plant. However, it will be understood that treatment may alternatively involve multiple applications of the same vector or composition or indeed combinations of the modified FoMV vectors disclosed herein. Where multiple (i.e. two or more) different vectors are applied to the same plant, these may be applied simultaneously, separately (in any order) or sequentially. The vectors may be delivered into the recipient plant by any combination of the above methods.

In a further alternative, vectors of the invention may be delivered into the recipient plant by rub inoculation of plant leaves with sap prepared from agroinoculated Nicotiana benthamiana leaves (a laboratory dicot host).

Accordingly, the invention includes a method wherein the modified FoMV is in the form of a DNA vector or an FoMV expression construct and comprised in Agrobacterium sp. and wherein a dicotyledonous plant, or the plant part or plant cell is of a dicotyledonous plant is transfected with the Agrobacterium; preferably wherein the dicotyledonous plant is Nicotiana sp.; more preferably Nicotiana benthamiana, or parts or cells thereof. This provides a way of increasing the supply of modified FoMV for the subsequent infection of monocot species, e.g. wheat or maize.

Also included is a method in which a monocotyledonous plant is infected with modified FoMV virus particles, modified FoMV DNA or FoMV RNA obtained from the Agrobacterium infected dicotyledonous plant as hereinbefore described.

Plant Growth Conditions

Where a plant is infected with a modified FoMV as described herein, the method may suitably comprise growing the infected plant in a photoperiod of 16 h light/8 h dark. Alternatively, as desired and depending on the plant species being infected and depending on the desired application (for example to prevent flowering), the infected plant may suitably be grown under 16 h light/8 h dark, 15 h light/9 h dark, 14 h light/10 h dark, 13 h light/11 h dark, 12 h light/12 h dark, 11 h light/13 h dark, 10 h light/14 h dark, 9 h light/15 h dark or 8 h light/16 h dark. Preferably, the infected plant may suitably be grown under a photoperiod of 16 h light/8 h dark

Temperature in the Light

Where the method comprises infecting a plant with a modified FoMV as described herein, the method comprises growing the recipient plant at a temperature in the light in the range 20° C. to 40° C., optionally in the range 21° C. to 40° C., 22° C. to 40° C., 23° C. to 40° C., 24° C. to 40° C., 25° C. to 40° C., 26° C. to 40° C., 27° C. to 40° C., 28° C. to 40° C., 29° C. to 40° C., 30° C. to 40° C., 31° C. to 40° C., 32° C. to 40° C., 33° C. to 40° C., 34° C. to 40° C., 35° C. to 40° C., 36° C. to 40° C., 37° C. to 40° C., 38° C. to 40° C. or 39° C. to 40° C. Suitably, where the method comprises infecting a plant with a modified FoMV as described herein, the method may optionally comprise growing the recipient plant at a temperature in the light in the range 20° C. to 39° C., 20° C. to 38° C., 20° C. to 37° C., 20° C. to 36° C., 20° C. to 35° C., 20° C. to 34° C., 20° C. to 33° C., 20° C. to 32° C., 20° C. to 31° C., 20° C. to 30° C., 20° C. to 29° C., 20° C. to 28° C., 20° C. to 27° C., 20° C. to 26° C., 20° C. to 25° C., 20° C. to 24° C., 20° C. to 23° C., 20° C. to 22° C. or 20° C. to 21° C. Commonly, where the method comprises infecting a plant with a modified FoMV as described herein, the method may optionally comprise growing the recipient plant at a temperature in the light in the range 21° C. to 39° C., 22° C. to 38° C., 23° C. to 37° C., 24° C. to 36° C., 25° C. to 35° C., 26° C. to 34° C., 27° C. to 33° C., 28° C. to 32° C., 29° C. to 31° C.

Although it will be appreciated that localised and systemic protein expression in a recipient plant may be achieved using a range of growth conditions, it has been discovered that the modified FoMV vectors described herein are particularly effective at driving protein expression in a recipient plant, at temperatures in the light exceeding 24° C. In particular, both the level and extent of protein expression in the recipient plants may be improved by growth at elevated temperatures in the light. Advantageously, therefore, where the method comprises infecting a plant with a modified FoMV as described herein, the method preferably comprise growing the recipient plant at a temperature in the light of 26.0° C., 26.1° C., 26.2° C., 26.3° C., 26.4° C., 26.5° C., 26.6° C., 26.7° C., 26.8° C., 26.9° C., 27.0° C., 27.1° C., 27.2° C., 27.3° C., 27.4° C., 27.5° C., 27.6° C., 27.7° C., 27.8° C., 27.9° C., 28.0° C., 28.1° C., 28.2° C., 28.3° C., 28.4° C., 28.5° C., 28.6° C., 28.7° C., 28.8° C., 28.9° C., 29.0° C., 29.1° C., 29.2° C., 29.3° C., 29.4° C., 29.5° C., 29.6° C., 29.7° C., 29.8° C., 29.9° C. or 30.0° C.

Temperature in the Dark

Where the method comprises infecting a plant with a modified FoMV as described herein, the method comprises growing the recipient plant at a temperature in the dark in the range 20° C. to 40° C., optionally in the range 21° C. to 40° C., 22° C. to 40° C., 23° C. to 40° C., 24° C. to 40° C., 25° C. to 40° C., 26° C. to 40° C., 27° C. to 40° C., 28° C. to 40° C., 29° C. to 40° C., 30° C. to 40° C., 31° C. to 40° C., 32° C. to 40° C., 33° C. to 40° C., 34° C. to 40° C., 35° C. to 40° C., 36° C. to 40° C., 37° C. to 40° C., 38° C. to 40° C. or 39° C. to 40° C. Suitably, where the method comprises infecting a plant with a modified FoMV as described herein, the method may optionally comprise growing the recipient plant at a temperature in the dark in the range 20° C. to 39° C., 20° C. to 38° C., 20° C. to 37° C., 20° C. to 36° C., 20° C. to 35° C., 20° C. to 34° C., 20° C. to 33° C., 20° C. to 32° C., 20° C. to 31° C., 20° C. to 30° C., 20° C. to 29° C., 20° C. to 28° C., 20° C. to 27° C., 20° C. to 26° C., 20° C. to 25° C., 20° C. to 24° C., 20° C. to 23° C., 20° C. to 22° C. or 20° C. to 21° C. Commonly, where the method comprises infecting a plant with a modified FoMV as described herein, the method may optionally comprise growing the recipient plant at a temperature in the dark in the range 21° C. to 39° C., 22° C. to 38° C., 23° C. to 37° C., 24° C. to 36° C., 25° C. to 35° C., 26° C. to 34° C., 27° C. to 33° C., 28° C. to 32° C., 29° C. to 31° C.

Although it will be appreciated that localised and systemic protein expression in a recipient plant may be achieved using a range of growth conditions, it has been discovered that the modified FoMV vectors described herein are particularly effective at driving protein expression in a recipient plant, at temperatures in the dark exceeding 21° C. In particular, both the level and extent of protein expression in the recipient plants may be improved by growth at elevated temperatures in the light. Advantageously, therefore, where the method comprises infecting a plant with a modified FoMV as described herein, the method preferably comprise growing the recipient plant at a temperature in the light of 21.0° C., 21.1° C., 21.2° C., 21.3° C., 21.4° C., 21.5° C., 21.6° C., 21.7° C., 21.8° C., 21.9° C., 22.0° C., 22.1° C., 22.2° C., 22.3° C., 22.4° C., 22.5° C., 22.6° C., 22.7° C., 22.8° C., 22.9° C., 23.0° C., 23.1° C., 23.2° C., 23.3° C., 23.4° C., 23.5° C., 23.6° C., 23.7° C., 23.8° C., 23.9° C., 24.0° C., 24.1° C., 24.2° C., 24.3° C., 24.4° C., 24.5° C., 24.6° C., 24.7° C., 24.8° C., 24.9° C. or 25.0° C.

In order to achieve improved level and extent of protein expression in the recipient plants, recipient (host) plants may be grown at a high light intensity. Typically, the intensity of light incident on a host plant may be measured micromoles (μmol) per square meter (m⁻²) per second (s⁻¹), (or μmol·m⁻²·s⁻¹) of Photosynthetically Active Radiation (PAR). PAR refers to the number of photons that may be used in photosynthesis that fall on a square metre every second. Preferably, a high light intensity may advantageously be combined with high temperature in the light. Preferably the light intensity will be 200 μmol·m⁻²·s⁻¹ to 2500 μmol·m⁻²·s⁻¹. Optionally the light intensity may be in the range 200 μmol·m⁻²·s⁻¹ to 2000 μmol·m⁻²·s⁻¹, 200 μmol·m⁻²·s⁻¹ to 1500 μmol·m⁻²·s⁻¹, 200 μmol·m⁻²·s⁻¹ to 1000 μmol·m⁻²·s⁻¹, 200 μmol·m⁻²·s⁻¹ to 750 μmol·m⁻²·s⁻¹, 200 μmol·m⁻²·s⁻¹ to 500 μmol·m⁻²·s⁻¹, 200 μmol·m⁻²s⁻¹ to 250 μmol·m⁻²·s⁻¹. Alternatively, the light intensity may be in the range 250 μmol·m⁻²·s⁻¹ to 2500 μmol·m⁻²·s⁻¹, 500 μmol·m⁻²·s⁻¹ to 2500 μmol·m⁻²·s⁻¹, 1000 μmol·m⁻²·s⁻¹ to 2500 μmol·m⁻²·s⁻¹, 1500 μmol·m⁻²·s⁻¹ to 2500 μmol·m⁻²·s⁻¹ or 2000 μmol·m⁻²·s⁻¹ to 2500 μmol·m⁻²·s⁻¹. Preferably, the light intensity may be in the range 250 μmol·m⁻²·s⁻¹ to 500 μmol·m⁻²·s⁻¹, 260 μmol·m⁻²·s⁻¹ to 300 μmol·m⁻²·s⁻¹. Ideally the light intensity will be approximately 250 μmol·m⁻²·s⁻¹.

Accordingly, the invention includes a method of transient, systemic overexpression of a protein in a plant comprising infecting or transfecting the plant with a modified FoMV virus or viral vector as hereinbefore described, or an FoMV expression construct or gRNA as hereinbefore described, and afterwards growing the plant, wherein the infection or transfection is carried out under a first set of environmental conditions and the growing of the plant is carried out under a second set of environmental conditions.

In preferred aspects, the second set of environmental conditions comprises a light/dark photoperiod, with a light temperature of greater than 21° C. and/or a dark temperature of greater than 24° C.; optionally wherein the light intensity is in the range 200 μmol·m⁻²·s⁻¹ to 2500 μmol·m⁻²·s⁻¹. In even more preferred aspect, the second set of environmental conditions is a 16 h light/8 h dark cycle with a temperature of about 26.7° C. in the light and about 21.1° C. in the dark and with a light intensity of about 250 μmol·m⁻²·s⁻¹ light.

Ideally, where the method comprises infecting a plant with a modified FoMV as described herein, the method may optimally comprise growing the recipient plants at 26.7° C. in the light and 21.1° C. in the dark, under 16 h light of high intensity (approximately 250 μmol·m⁻²·s⁻¹ light) and 8 h dark cycle.

Where the recipient plant is Triticum sp. for example Tiricicum aestivum, or Zea sp., for example Zea mays, preferably the plant will be grown under conditions of 16 h light/8 h dark. Preferably the plant will be grown under conditions of 26.7° C. in the light and 21.1° C. in the dark at approximately 50% relative humidity and a 16 h photoperiod (approximately 250 μmol·m⁻²·s⁻¹ light).

Where the infected plant is Nicotiana benthamiana the method may optimally comprise growing the recipient plants at 23° C. in the light and 20° C. in the dark, at 60% relative humidity and a 16 h photoperiod (approximately 130 μmol·m-2·s-1 light).

Accordingly, the invention provides a method, wherein the plant is a monocotyledonous plant, or the plant part or plant cell is of a monocotyledonous plant. Preferably, the plant is selected from, or the, plant part or plant cell is of a plant selected from the group consisting of Triticum sp. and Zea sp. More preferably, the plant is selected from, or the plant part or plant cell is of a Triticum aestivum plant.

Alternatively, the plant is a dicotyledonous plant, or the plant part or plant cell is of a dicotyledonous plant. For example, the plant may be, or the, plant part or plant cell is of a Nicotiana benthamiana plant.

In accordance with all aspects of the invention, the vectors and methods disclosed herein may be used to generate protein expression in any plant species, which may be monocots or dicots.

Accordingly, the invention provides a plant, plant part or plant cell infected with a modified FoMV as defined herein. That is, a plant, plant part or plant cell i.e. comprising genetic material comprising a polynucleotide of SEQ ID NO: 1, SEQ ID NO: 2, SEQ ID NO: 3, SEQ ID NO: 4; SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or variants thereof.

In the present invention, plant, plant material or plant part may refer to leaves, stems, roots, stalks, root tips, tissue or cells. Preferably, the plant, plant part or plant cell has increased expression of a desired polypeptide compared to a genetically equivalent but uninfected plant, plant part or plant cell. Typically, the leaves of the infected plant have increased expression of a desired polypeptide compared to those of a genetically equivalent but uninfected plant. The infection may be localised or systemic.

Preferably, the expression levels of a desired polypeptide, whether native to the recipient plant or heterologous, are increased in the plant infected with a modified FoMV as described herein, relative to an uninfected plant. The expression levels of the polypeptide or protein of interest may desirably be increased in the range 5% to 500% relative to uninfected plants; optionally the range 10% to 250%, 20% to 200% or 25% to 100%.

Optionally, the plant or plant is a monocotyledonous plant, or the plant part or plant cell is of a monocotyledonous plant. Appropriate recipient plants may include grasses, trees, crops, shrubs, vegetables and ornamentals. More particularly, plants suitable for infection with modified FoMV vectors of the invention are those which produce a high yield of grain for food, feedstock or biomass for fuel or paper production. Examples of suitable plant types include, but are not limited to fast growing crops, for example wheat, soybean, alfalfa, corn, rice, maize, sorghum, panicum oat, sugar cane and sugar beet. Preferably, the plant is a cereal crop selected from the genera Triticum, Zea, Oryza, Hordeum, Sorghum, Panicum, Avena, Saccharum or Secale. More preferably the plant is a Triticum sp. plant. Even more preferably the plant is a Triticum aestivum plant. Preferably, the modified plant, plant part or plant cell is, or is of a plant selected from the group consisting of Triticum sp. and Zea sp.

Commonly, the plant, plant part or plant cell is selected from Triticum sp., a synthetic hybrid wheat species, Aegilops sp., Hordeum sp., Oryza sp., Zea sp., Avena sp., Poa sp., Secale sp., Sorghum sp., Setaria sp., Panicum sp., Brachypodium sp., Agropyron sp., Lolium sp., Festuca sp., Agrostis sp., Miscanthus sp., Bromopsis sp., Buchloe sp., Bouteloua sp., or Triticosecale sp.

Optionally, the plant, plant part or plant cell may be selected from Setaria viridis or Setaria italica.

Preferably, the plant, plant part or plant cell may be selected from Triticum aestivum, Triticum durum, Triticum urartu, Triticum monococcum, Triticum boeoticum, Triticum turgidum, Triticum dicoccon, Triticum timopheevi, Triticum polonicum, Triticum turanicum, Triticum spelta, Triticum compactum, Triticum sphaerococcum, or Triticum carthlicum. Most preferably, the plant, plant, plant part or plant cell is, or is of a Triticum aestivum plant

Alternatively, the plant, plant part or plant cell may be selected from Aegilops tauschii, Aegilops bicornis, Aegilops biuncialis, Aegilops columnaris, Aegilops crassa, Aegilops cylindrica, Aegilops geniculata, Aegilops juvenalis, Aegilops mutica, Aegilops neglecta, Aegilops sharonensis, Aegilops speltoides, Aegilops triuncialis, Aegilops umbellulata, or Aegilops ventricosa.

Where the plant, plant, plant part or plant cell is a dicotyledonous plant, or the plant part or plant cell is of a dicotyledonous plant, preferably the plant, plant, plant part or plant cell is, or is of a Nicotiana benthamiana plant.

The invention also provides a composition comprising a modified FoMV as defined herein. Accordingly, such a composition may comprise a modified FoMV comprising a nucleotide sequence of any of SEQ ID NO: 1, SEQ ID NO: 2, SEQ ID NO: 3, SEQ ID NO: 4; SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or variants thereof as defined herein.

In accordance with methods of the invention, in use, the vectors or compositions disclosed herein are usually applied directly onto the surface of the plant or crop. Typically, this may be achieved by direct application to the plant or crop, for example by rub-inoculation or by spraying the vector or composition directly onto the plant material, for example onto leaves, stems or roots of the plant during vegetative phase although other equally feasible methods of application will be known in the art. It is envisaged that the compounds disclosed herein may also be applied indirectly to the medium (e.g. soil or water) in which the plants or crop are grown.

Infection of the plants in accordance with the methods of the invention may involve a single application of the vector or composition either to the plant or to the growth medium. However, it will be understood that treatment may alternatively involve multiple applications of the same vector or composition or indeed combinations of the vectors or compositions disclosed herein. Where multiple (i.e. two or more) different vectors or compositions are applied to the same plant, these may be applied simultaneously, separately (in any order) or sequentially.

The vectors or compositions disclosed herein are typically provided to the plant or crop in the form of an aqueous solution. However, the vectors or compositions disclosed herein may also be provided to the plant or crop in solid form such as a powder, dust or in granular form and combinations thereof.

The invention provides the use of a composition comprising any of the vectors disclosed herein (and combinations thereof) in expressing protein in a plant. Accordingly, the vectors or compositions disclosed herein are preferably to be applied to the plant or growth medium as an aqueous solution.

The invention also includes any method for improving plants of any kind, whether agricultural or horticultural crop species, by identifying monocotyledonous plants for suitability for a breeding process, comprising infecting or transfecting a monocot plant with a modified FoMV virus as hereinbefore defined, or an FoMV expression construct or gRNA as hereinbefore defined, growing the plant for a period of time and then examining the plant for the presence or absence of one or more phenotypic, biochemical and/or genetic characteristics as a measure of said suitability.

For convenience, vectors or compositions disclosed herein may be combined with other active ingredients used for the treatment of plants, for example they may be incorporated into other agrochemical products such as fertilisers, herbicides, anti-bacterial or anti-fungal agents and/or pesticides.

### BRIEF DESCRIPTION OF THE DRAWINGS

Embodiments of the invention are further described hereinafter with reference to the accompanying drawings, in which:

FIG. 1 shows an alignment of the duplicated subgenomic promoter 2 (sgp2) sequences used for expression of heterologous proteins. 1. to 5. 170 nt-, 101 nt-, 90 nt-, 55 nt- and 45 nt-long sgp2 sequence duplications used in the modified FoMV vectors. Fragments of the viral genes TGB3, ORF 5A and CP spanned by the different sgp2 duplications are boxed and displayed underneath each sequence. The “core sequence” is an octa-nucleotide sequence (GTTAGGGT) which is found across different FoMV isolates, and is essential for sgp2 activity. Vertical arrows indicate two single nucleotide polymorphisms between the sgp2 sequence duplication used by Liu and colleagues and the sgp2 duplications we produced. Liu et al. (2016) used a different FoMV isolate, “Robertson” vs pCF, for their VIGS vector development.

FIG. 2 shows polynucleotide sequences of the duplicated sgp2 used for expression of heterologous proteins.

FIG. 3 shows levels of heterologous gene expression obtained from the modified FoMV vectors.

FIG. 4 shows heterologous expression of Green Fluorescent Protein (GFP) in N. benthamiana using the developed modified FoMV vectors. FIG. 4 A shows immuno-detection of GFP and FoMV coat protein (CP) by Western-blotting from infiltrated leaf samples collected 3 days post infiltration (dpi). p19 is a suppressor of RNA silencing from Tomato bushy stunt virus, which was coinfiltrated with each vector. pCF is the non-modified virus. sgp2-101 is an empty modified FoMV vector containing a 101 nt-long duplicated sgp2 promoter sequence. sgp2/101-GFP, sgp2/90-GFP, sgp2/55-GFP and sgp2/45-GFP are modified FoMV vectors where GFP is expressed under the control of the duplicated 101-, 90-, 55- or 45-nt long sgp2 promoter sequences, respectively. Ponceau-stained protein gel panels show equal protein loading between the samples. FIG. 4B shows sections of infiltrated leaves observed using stereomicroscopy under bright field and UV light at 3-dpi. FIG. 4C shows a section of upper non-infiltrated leaf observed using stereomicroscopy under bright field and UV light at 21-dpi. GFP-expressing areas are delimited by white lines for clarity.

FIG. 5 shows heterologous expression of GFP in maize (Zea mays) using the newly developed modified FoMV vector sgp2/101-GFP. Sections of rub-inoculated (leaf 3) and upper non-inoculated leaves (leaves 4, 5, 6) observed using stereomicroscopy under bright field and UV light at 13-dpi are shown.

FIG. 6 shows heterologous expression of GFP in bread wheat (Triticum aestivum) using the developed modified FoMV vector sgp2/101-GFP. Panel A shows sections of rub-inoculated leaves (leaf 1, 2) observed using stereomicroscopy under bright field and UV light at 8-dpi. Panel B shows sections of upper non-inoculated fourth leaves observed using stereomicroscopy under bright field and UV light at 26-dpi. Fourth leaves of two plants (plant 1, 2) are shown.

FIG. 7 shows heterologous expression achieved in wheat under different growth conditions, using the pCF.sgp2/101 vector

−: No detectable gene expression
+: Low expression level, with gene expressing areas being very limited in size and number
++: High expression level with gene expressing areas being numerous and wide

FIG. 8 shows heterologous expression levels achieved in wheat using the pCF.sgp2/101, PV139wa.sgp2/101 and PV139wa.sgp2/170 vectors

+: Low expression level
++: High expression level
x: Limited spread. Gene expressing areas are limited in size and number
xx: Medium spread. Gene expressing areas can be numerous in some parts of the leaf
xxx: Gene expressing areas are numerous and nearly uniformly spread along the leaf width and/or length.

FIG. 9 shows micrographs and Western blots of samples involving FoMV-mediated expression of GFP in Agro-infiltrated Nicotiana benthamiana leaves.

FIG. 10 shows PV101gw-mediated expression of the green fluorescent protein (GFP) in Nicotiana benthamiana.

FIG. 11 shows micrographs of FoMV-mediated expression of a necrotrophic fungal effector, ToxA from Stagonospora nodorum. A=ToxA-expressing FoMV (PV101-SnToxA) was rub-inoculated to a ToxA-sensitive (Halberd, Hal) and a ToxA-insensitive wheat cultivar (Chinese Spring, CS). Plants 13 day-old were inoculated and then inoculated leaves 2 were observed at 6 dpi. PV101-GFP was used as a control. The white bar represents 20 mm. B=FoMV-expressing full-length ToxA (PV101-SnToxA) or a non-secreted version of ToxA (PV101-SnToxA_noSP) were inoculated to 10 day-old wheat plants. First systemic leaves (3rd leaves) were observed at 11 dpi and 16 dpi under a fluorescence stereomicroscope mounted with a longpass filter (LP). Photographs taken at 16 dpi are from the same leaf areas as those shown at 11 dpi. White bars represent 2.5 mm.

FIG. 12 shows micrographs of FoMV-mediated expression of a ca. 600 aa-long protein, GUSPlus. GUSPlus-expressing FoMV (PV101-GUSPlus) was rub-inoculated onto wheat cvs. Pakito (A) and Riband (B) and onto maize plants (line B73, C). The empty vector PV101 was used as a control. GUSPlus activity in leaf samples was detected by histochemical staining with X-Gluc. Inoculated leaves taken at 9 dpi were cut and stained. Systemic leaves taken at either 15 dpi (1st) or 22 dpi (2nd and 3rd) were cut and then stained. Each leaf piece comes from a different individual plant. Black bars represent 20 mm.

FIG. 13 shows the nucleotide sequence of the Taiwanese FoMV isolate pCF, with no modifications [SEQ ID NO: 8].

FIG. 14 shows the nucleotide sequence of the wheat-adapted isolate PV139wa, with no modifications [SEQ ID NO: 9].

FIG. 15 shows the nucleotide sequence of the vector pCF.sgp2/45-GFP SEQ ID NO: 10] where the duplicated sgp2 is 45 nt-long (duplicated regions are in highlighted in grey), and where the GFP coding sequence is underlined.

FIG. 16 shows the nucleotide sequence of the vector pCF.sgp2/55-GFP [SEQ ID NO: 11] where the duplicated sgp2 is 55 nt-long (duplicated regions are in highlighted in grey), and where the GFP coding sequence is underlined.

FIG. 17 shows the nucleotide sequence of the vector pCF.sgp2/90-GFP [SEQ ID NO: 12] where the duplicated sgp2 is 90 nt-long (duplicated regions are in highlighted in grey), and where the GFP coding sequence is underlined. The start codon which is present in the duplicated region was mutated by a T to G substitution in the first repeat of the duplicated sequence. The mutated nucleotide is in lower case.

FIG. 18 shows the nucleotide sequence of the vector pCF.sgp2/101-GFP [SEQ ID NO: 13] where the duplicated sgp2 is 101 nt-long (duplicated regions are in highlighted in grey), and where the GFP coding sequence is underlined. The start codon which is present in the duplicated region was mutated by a T to G substitution (shown in lowercase) in the first repeat of the duplicated sequence.

FIG. 19 shows the nucleotide sequence of the vector PV139wa.sgp2/101-GFP [SEQ ID NO: 14] where the duplicated sgp2 is 101 nt-long (duplicated regions are in highlighted in grey), and where the GFP coding sequence is underlined. The start codon which is present in the duplicated region was mutated by a T to G substitution in the first repeat of the duplicated sequence. The mutated nucleotide is in lower case.

FIG. 20 shows the nucleotide sequence of the vector PV139wa.sgp2/170-GFP [SEQ ID NO: 15] (170-nt long sgp2 duplication). Duplicated regions highlighted in grey. GFP coding sequence is underlined.

### DETAILED DESCRIPTION

### Example 1: General Materials and Methods

Plants and Growth Conditions

Nicotiana benthamiana plants were grown in a controlled environment room with day/night temperature of 23° C./20° C. at 60% relative humidity and a 16 h photoperiod (approximately 130 μmol·m-2·s-1 light).

Maize (Zea mays, line B73) and bread wheat (Triticum aestivum, cv. Riband) plants were grown in a controlled environment cabinet with day/night temperature of 26.7° C./21.1° C. at approximately 50% relative humidity and a 16 h photoperiod (approximately 250 μmol·m-2·s-1 light).

Vector Construction

All Polymerase Chain Reactions (PCRs) were carried out using the high fidelity DNA polymerase Phusion™ (New England Biolabs) unless otherwise stated. All the constructs described below were verified by sequencing. Oligonucleotide sequences used in the construction of vectors are listed in Table 1.

Plasmids

The plasmid pCF containing the genome of Foxtail mosaic virus (FoMV) isolate pCF cloned under the control of Cauliflower mosaic virus (CaMV) 35S promoter was a gift from Ming-Ru Liou and Yau-Heiu Hsu (National Chung Hsing University, Taichung, Taiwan).

Sequence of the Taiwanese FoMV isolate pCF, with no modifications is provided as SEQ ID NO: 8. Many of the vectors described herein derive from this isolate.

The binary vector pGR106 is an agroinfection vector containing a modified Potato virus X genome with a duplicated subgenomic promoter, cloned between CaMV 35S promoter and the nos (nopaline synthase gene from Agrobacterium tumefaciens) terminator sequence (Lu et al., EMBO J, 2003).

The vector pActIsGFP is a vector containing the coding sequence of an improved version of Green Fluorescent Protein (GFP S65T; Heim et al., Nature, 1995).

The vector pBIN61-P19 was a gift from Patrice Dunoyer (Institut de Biologie Moléculaire des Plants, CNRS—University of Strasbourg, France). This is a binary plasmid for expression of the p19 protein from Tomato bushy stunt virus, a well-known suppressor of gene silencing often used to ensure high levels of heterologous protein production in planta.

Construction of a binary pGR-FoMV.pCF plasmid containing full length genome of FoMV, isolate pCF

The genome of FoMV isolate pCF was cloned into a binary vector as follows. CaMV 35S promoter sequence was amplified by PCR from the vector pGR106 with the oligonucleotides 5′35Sp and 5′FoMV-3′35Sp. The 5′-part of FoMV isolate pCF was amplified by PCR from the plasmid pCF with the oligonucleotides 3′35Sp-5′FoMV and SpeI-FoMV1040R. The two resulting amplicons were fused by PCR with the oligonucleotides 5′35Sp and SpeI-FoMV1040R, using a 38-nt long complementary region which was artificially introduced at the 3′-extremity of 35S promoter and at the 5′-extremity of FoMV amplicons. The resulting 35S-5′-FoMV fragment was then cloned between EcoRV SpeI recognition sites into EcoRV+SpeI-digested pGR106 vector backbone to produce the plasmid pGR-5′-pCF. Finally, the 3′-part of FoMV pCF was obtained by BlpI+XbaI digestion of pCF plasmid and this fragment was then inserted into the BlpI+SpeI-digested pGR-5′-pCF. The resulting construct was named pGR-FoMV.pCF. In this binary plasmid the FoMV genome is under control of CaMV 35S promoter, and is flanked by the nos terminator sequence at the 3′-end.

Construction of a binary FoMV vector pGR-FoMV.pCF.sgp2/101-GFP for expression of green fluorescent protein (GFP)

To avoid doing all cloning steps with the full-length pGR-FoMV.pCF plasmid (10.8 kbp), successive insertions of a duplicated promoter 2 (sgp2) and then of the GFP coding gene were done into a smaller “shuttle” vector of about 4 kbp. This vector contains the FoMV 3′-part from the middle of TGB2 coding gene up to the end of the virus 3′-end. The non-modified sgp2 promoter is thus present in this plasmid. After assembly, the sequence “sgp2-GFP-FoMV 3′-part” was cloned back into the pGR-FoMV.pCF binary vector.

The nucleotide sequence of the vector pCF.sgp2/101-GFP where the duplicated sgp2 is 101 nt-long is provided as SEQ ID NO: 13 (duplicated regions are in highlighted in grey), and where the GFP coding sequence is underlined. The start codon which is present in the duplicated region was mutated by a T to G substitution (shown in lowercase) in the first repeat of the duplicated sequence:

Construction of the shuttle vector pB-Fmcs-10

A pBxs plasmid was created by introducing SpeI and XhoI restriction sites into pMA-RQ (ampR)pMA-T plasmid by PCR with the oligonucleotides pBxs-fw and pBxs-rev.

A fragment corresponding to FoMV pCF 3′-part was obtained by SpeI+XhoI digestion of plasmid pCF and then cloned into SpeI+XhoI-digested-pBxs to give the plasmid called pB-F. A multi cloning site was then inserted into pB-F by two successive PCRs with the oligonucleotides pB-Fmcs-10-fw1 and pB-Fmcs-10-rev1 for the first reaction, and pB-Fmcs-10-fw2 and pB-Fmcs-10-rev2 for the second reaction.

Construction of pB-Fsgp2/101-GFP

A 101 nt-long sequence of subgenomic promoter 2 and the GFP coding sequence were inserted into the shuttle vector as follows. A sequence corresponding to the 101-nt long variant of predicted FoMV pCF subgenomic promoter 2 (sgp2/101) was produced by gene synthesis and cloned into SphI+AscI-digested pB-Fmcs-10 to give the plasmid pB-Fsgp2/101. GFP S65T coding sequence was amplified by PCR with oligonucleotides GFP5′-ClaI-fw and GFP3′-XbaI-rev from the plasmid pActIsGFP and cloned into ClaI+XbaI-digested pB-Fsgp2/101 to give pB-Fsgp2/101-GFP. This plasmid contains a “sgp2/101-GFP-FoMV 3′-part” sequence.

Construction of pGR-FoMV.pCF.sgp2/101-GFP

The sequence “sgp2/101-GFP-FoMV 3′-part” from the shuttle vector pB-Fsgp2/101-GFP was inserted into pGR-FoMV.pCF as follows.SpeI+XhoI-digested pB-Fsgp2/101-GFP (FoMV fragment) was cloned into SpeI+XhoI-digested pGR-FoMV.pCF to give the plasmid pGR-FoMV.pCF.sgp2/101-GFP. The corresponding empty vector pGR-FoMV.pCF.sgp101 (a vector with the duplicated sgp2 but without the GFP coding gene) was constructed by cloning SpeI+XhoI-digested pB-Fsgp101 (FoMV fragment) into SpeI+XhoI-digested pGR-FoMV.pCF.

Construction of pGR-FoMV.pCF.sgp2/90-GFP, pGR-FoMV.pCF.sgp2/55-GFP and pGR-FoMV.pCF.sgp2/45-GFP

Three other FoMV vectors similar to pGR-FoMV.pCF.sgp2/101-GFP except that they contain different fragments of predicted FoMV pCF sgp2 of 90 nts, 55 nts and 45 nts in size were constructed. The cloning steps to assemble these vectors, though very similar to the steps described in the part about pGR-FoMV.pCF.sgp2/101-GFP, involved another shuttle vector called pB-Fmcs.

Construction of pB-Fmcs

A multiple cloning site (MCS) was inserted into pB-F (see paragraph B. 3. a.) by two successive PCRs with the oligonucleotides pB-Fmcs-fw1 and pB-Fmcs-rev1 for the first reaction, and pB-Fmcs-fw2 and pB-Fmcs-rev2 for the second reaction.

Construction of pB-Fsgp2/90-GFP, pB-Fsgp2/55-GFP and pB-Fsgp2/45-GFP

The three variants of sgp2 and the GFP coding sequence were inserted into the shuttle vector pB-Fmcs as follows. Sequences corresponding to the 90-, 55- and 45-nt long fragments of predicted FoMV pCF sgp2 (sgp2/90, sgp2/55 and sgp2/45, respectively) were synthesised commercially and cloned into SphI+AscI-digested pB-Fmcs to give the plasmids pB-Fsgp90, pB-Fsgp55 and pB-Fsgp45, respectively. GFP S65T coding sequence was then amplified by PCR from the plasmid pActIsGFP with oligonucleotides GFP5′-ClaI-fw and GFP3′-XbaI-rev and cloned into ClaI+XbaI-digested pB-Fsgp2/90, pB-Fsgp2/55 and pB-Fsgp2/45 to give pB-Fsgp2/90-GFP, pB-Fsgp2/55-GFP and pB-Fsgp2/45-GFP, respectively. These plasmids contain a “sgp2/90- or sgp2/55- or sgp2/45-GFP-FoMV 3′-part” sequence.

Construction of pGR-FoMV.pCF.sgp90-GFP, pGR-FoMV.pCF.sgp55-GFP and pGR-FoMV.pCF.sgp45-GFP

The “sgp2/90- or sgp2/55- or sgp2/45-GFP-FoMV 3′-part” sequences were cloned back into pGR-FoMV.pCF as follows. The FoMV fragments of SpeI+XhoI-digested pB-Fsgp2/90-GFP, pB-Fsgp2/55-GFP and pB-Fsgp2/45-GFP were inserted into SpeI+XhoI-digested pGR-FoMV.pCF to give the plasmids pGR-FoMV.pCF.sgp2/90-GFP, pGR-FoMV.pCF.sgp2/55-GFP and pGR-FoMV.pCF.sgp2/45-GFP, respectively. The corresponding empty vectors pGR-FoMV.pCF.sgp2/90, pGR-FoMV.pCF.sgp2/55 and pGR-FoMV.pCF.sgp2/45 were constructed by cloning the FoMV fragments of SpeI+XhoI-digested pB-Fsgp2/90, pB-Fsgp2/55 and pB-Fsgp2/45, respectively, into SpeI+XhoI-digested pGR-FoMV.pCF.

5. Construction of the Wheat Adapted Isolate

The FoMV sequence from which the vectors have been developed derives from the isolate PV139 obtained from Jeff Ackerman and Dallas Seifers (KSU). All the isolates were a gift. Wheat adapted isolate was constructed as follows; freeze-dried PV139-infected sorghum leaf tissue was used to infect wheat plants (cvs. Chinese Spring, Riband and Bobwhite). Systemic and symptomatic wheat leaves were collected and used for serial passaging of the virus in order to adapt it to wheat. After six passages, total RNA was extracted from systemic and symptomatic wheat leaves and then small-RNA sequenced. The master genome of the wheat adapted FoMV-PV139 was obtained by aligning the small RNA reads to a FoMV template sequence (FoMV sequence of the p9 clone, obtained from Nancy Robertson). The wheat-adapted FoMV-PV139 clone was then created by gene synthesis and restriction cloning assembly.

The nucleotide sequence of the wheat adapted isolate PV139wa, with no modifications is provided as SEQ ID NO: 9.

The nucleotide sequence of the wheat adapted isolate PV139wa, with sgp2 duplicated 101 nt insert (PV139wa.sgp2/101) is provided as SEQ ID NO: 14.

The nucleotide sequence of the wheat adapted isolate PV139wa, comprising a 170 nt long sgp2 duplication (PV139wa.sgp2/170) is provided as SEQ ID NO: 15. Duplicated regions are in highlighted in grey and the GFP coding sequence is underlined.

Delivery of the FoMV Vectors to Host Plants

Nicotiana benthamiana

FoMV vectors were delivered to N. benthamiana by agroinfiltration. FoMV vectors or pBIN61-P19 plasmid were introduced into Agrobacterium tumefaciens GV3101 pCH32 pSa-Rep by electroporation. Transformants were selected on Luria-Bertani (Lennox) agar plates supplemented with gentamycin (25 μg/mL) and kanamycin (50 μg/mL) after ≥48 h incubation at 28° C. Single colonies were streaked and grown in liquid Luria-Bertani (Lennox) medium supplemented with antibiotics at 28° C. for 20 h under constant shaking (250 rpm). Agrobacterium cultures were then pelleted at 2013×g for 20 min at 17° C. After having discarded the supernatant, the cells were resuspended in induction medium (10 mM 2-(N-Morpholino)ethanesulfonic acid, 10 mM MgCl2 and 100 μM acetosyringone), adjusted at 1.2-1.5 OD600 and incubated at room temperature for h. Each FoMV vector-containing Agrobacterium culture was mixed with an equal volume of pBIN61-P19-containing Agrobacterium culture diluted at a similar OD600 and then pressure infiltrated to the abaxial face of leaves from 6 to 8 leaf-stage N. benthamiana plants using needleless syringe.

Bread Wheat (Triticum aestivum) and Maize (Zea mays)

FoMV vectors were delivered to young wheat and maize plants by rub inoculation onto the leaves. Virus-containing sap was prepared from FoMV-vector agroinfiltrated N. benthamiana leaves collected at 6 to 7 days post-infiltration. Leaves were ground in 0.67 w/v deionized water and then supplemented with 1% (w/v) celite. Sap was then rub-inoculated onto the first two leaves of 10-11 day-old wheat plants (cv. Riband; 2 leaf-stage) or the second and third leaves of 10 day-old maize plants (line B73; 3 leaf-stage). After inoculation, the plants were left for 5 min and then sprayed with water to clean up the leftover sap and celite present on the inoculated leaves. The inoculated plants were then covered with lids or autoclave bags in order to keep the humidity content high, and kept under low light conditions (usually under a growing shelf) for about 24 h before being returned to standard growth conditions.

Assessment of Heterologous Expression

Immuno-Detection of GFP and FoMV Coat Protein (FoMV-CP)

50-100 mg of N. benthamiana leaf samples were put in 2 ml safe-lock micro-tubes with 0.17 w/vol suspension buffer (100 mM Tris-HCl, pH 8 1 mM DL-Dithiothreitol) and 3 chrome beads (3 mm diameter) and then homogenized for 2×20 s at 1750 rpm in a tissue homogenizer (2010 Geno/Grinder, SPEX SamplePrep). 100 μl of leaf extract were supplemented with 33 μl 4× Laemmli extraction buffer (8% SDS, 20% 2-mercaptoethanol, 40% glycerol, 0.008% bromophenol blue, 0.25 M Tris-HCl pH 6.8) and incubated at 95° C. for 5 min to allow denaturation of proteins. The samples were spun down (centrifuged) at 16100×g for 5 min to pellet any cell debris and the supernatants were loaded onto a 16% SDS-polyacrylamide gel. Proteins were separated by electrophoresis performed in 25 mM Tris, 192 mM glycine, 0.1% (w/v) SDS and then electrotransferred to a nitrocellulose membrane (Amersham Protran Premium 0.45 NC, GE Healthcare Life Sciences) for 90 min at 90 V in 25 mM Tris, 192 mM glycine, 20% (v/v) methanol. After transfer, membranes were rinsed in PBS-T buffer (50 mM Tris, 150 mM NaCl and 0.1% (w/v) Tween 20) for 5 min and then blocked in PBS-T+5% (w/v) dry milk for 45 min at room temperature and under constant shaking. Blocked membranes were incubated overnight at 4° C. under constant shaking with primary antibodies diluted in PBS-T+5% dry milk. The rabbit anti GFP antibody (ref G10362, Life Technologies) was diluted at 1:200 and the rabbit anti FoMV-CP antibody (ChinaPeptides) was diluted at 1:10000. Non-bound antibodies were eliminated by rinsing the membranes three times in PBS-T for 15 min at room temperature under constant shaking. Incubation with secondary antibody (goat anti-rabbit-peroxidase antibody, ref A0545, Sigma-Aldrich) diluted at 1:10000 in PBS-T+5% dry milk was performed for 3 hours at room temperature under constant shaking. Non-bound antibodies were removed by rinsing the membranes three times in PBS-T for 10 min at room temperature under constant shaking. Membrane-bound immune complexes were revealed with Amersham ECL Prime kit (GE Healthcare Life Sciences). Chemiluminescence signals were visualised using X-ray films (Hyperfilm ECL, GE Healthcare Life Sciences). Protein loading was then estimated by staining the membranes in Ponceau S (Sigma-Aldrich) solution for 5 min (5% acetic acid (v/v) 0.1% Ponceau S (w/v)). Excess of Ponceau S was eliminated by soaking the stained membranes into deionized water for about 1 min under constant shaking.

Stereomicroscopy

GFP-induced fluorescence in plant samples was visualised with a Leica M205 FA stereomicroscope using a GFP3 filter (excitation filter: 450-490 nm; barrier filter 500-550 nm). Pictures were taken using Leica LAS AF software.

TABLE 1
Oligonucleotide sequences used in the
construction of vectors

| | | |
|---|---|---|
| 5′35Sp | CTCGCATGCCTGCAGGTC | [SEQ ID |
| | | NO: 16] |
| 5′FoMV- | CGGTTTCGGAAGAGTTTTCCCTCTCCAAAT | [SEQ ID |
| 3′35Sp | GAAATGAA | NO: 17] |
| 3′35Sp- | TTCATTTCATTTGGAGAGGGAAAACTCTTC | [SEQ ID |
| 5′FoMV | CGAAACCG | NO: 18] |
| Spel- | TATCACTAGTGGTTTGCCTCAGCTTAGC | [SEQ ID |
| FoMV1040R | | NO: 19] |
| pBxs-fw | ATACTCGAGGCGTGGATCCCGCTGGGCCTC | [SEQ ID |
| | | NO: 20] |
| pBxs-rev | GTTATACTAGTTTTTTCCTTATGCGGCCTT | [SEQ ID |
| | | NO: 21] |
| pB-Fmcs- | CAGTTAACTTACTCTAGACACTCAACGACC | [SEQ ID |
| 10-fw1 | GCATTGAG | NO: 22] |
| pB-Fmcs- | GTCGACTTGTTCGGCCGAGTGCCAGCAGTT | [SEQ ID |
| 10-rev1 | TCCGGT | NO: 23] |
| pB-Fmcs- | AGTTGGCGCGCCAATCAGTTAACTTACTCT | [SEQ ID |
| 10-fw2 | AGACA | NO: 24] |
| pB-Fmcs- | ATCGATAGCTGTCGACTTGTTCGGCCGAG | [SEQ ID |
| 10-rev2 | | NO: 25] |
| pB-Fmcs- | CAGTTAACTTACTCTAGACGCATTGAGGGT | [SEQ ID |
| fw1 | GTTAGGG | NO: 26] |
| pB-Fmcs- | GTCGACTTGTTCGGCCGTCGTTGAGTGGAG | [SEQ ID |
| rev1 | TGCCAG | NO: 27] |
| pB-Fmcs- | AGTTGGCGCGCCAATCAGTTAACTTACTCT | [SEQ ID |
| fw2 | AGACG | NO: 28] |
| pB-Fmcs- | ATCGATAGCTGTCGACTTGTTCGGCCGTC | [SEQ ID |
| rev2 | | NO: 29] |
| GFP5′- | AGGTCAATCGATATGGTGAGCAAGGGCGAG | [SEQ ID |
| ClaI-fw | G | NO: 30] |
| GFP3′- | TATGCTTCTAGATTACTTGTACAGCTCGTC | [SEQ ID |
| XbaI-rev | CATG | NO: 31] |

### Example 2: Sgp2 Promoter Sequences Used to Drive Expression of Proteins in Plants

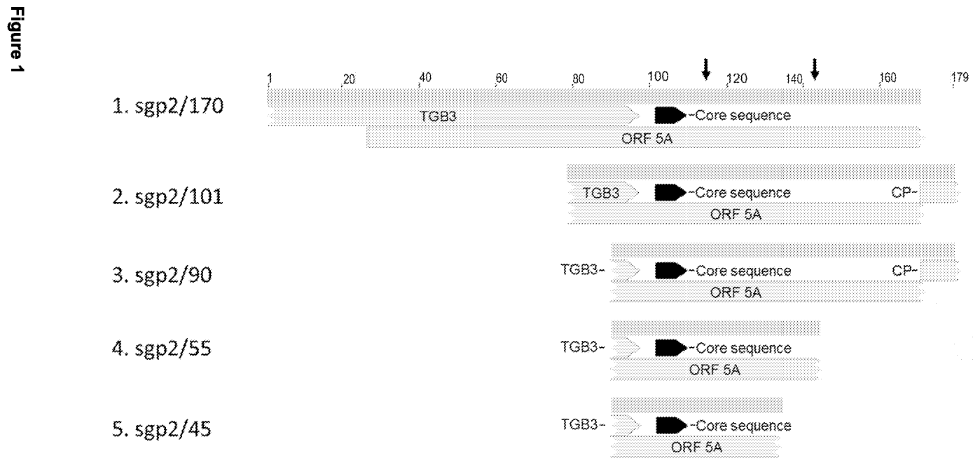

FIG. 1 shows an alignment of the duplicated subgenomic promoter 2 (sgp2) sequences used for expression of proteins in plants. Sequence 1 refers to a 170 nt-long sgp2 duplication originally used by Liu and colleagues (Liu et al. 2016, Plant Physiology 171:1801-1807) in their FoMV-VIGS (virus-induced gene silencing) vector. Sequences 1 to 5 refer to 170 nt-, 101 nt-, 90 nt-, 55 nt- and 45 nt-long sgp2 sequence duplications used in our modified FoMV vectors for protein expression respectively. Fragments of the viral genes TGB3, ORF 5A and CP spanned by the different sgp2 duplications are boxed and displayed underneath each sequence. The “core sequence” is an octa-nucleotide sequence (GTTAGGGT) which is found across different FoMV isolates, and it is thought to be essential for sgp2 activity. Vertical arrows indicate two single nucleotide polymorphisms between the sgp2 sequence duplication used by Liu and colleagues and the sgp2 duplications we produced. Liu et al. (2016) and us used different FoMV isolates, “Robertson” vs pCF, for their VIGS vector development.

### Example 3: Polynucleotide Sequences of the Duplicated Sgp2 Used for Expression of Heterologous Proteins

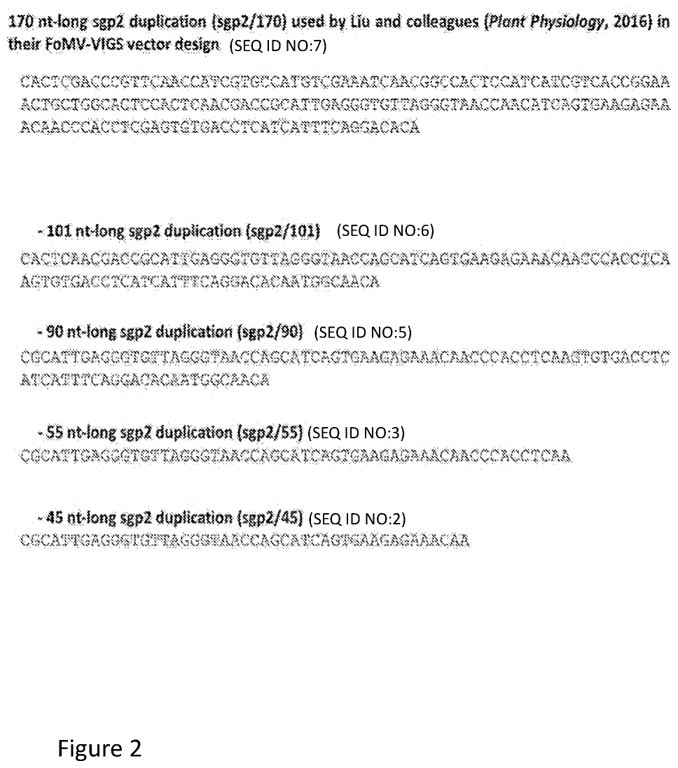

Polynucleotide sequences of the duplicated sgp2 used for expression of heterologous proteins are shown in FIG. 2.

### Example 4: Levels of Heterologous Gene Expression Obtained from the Modified FoMV Vectors

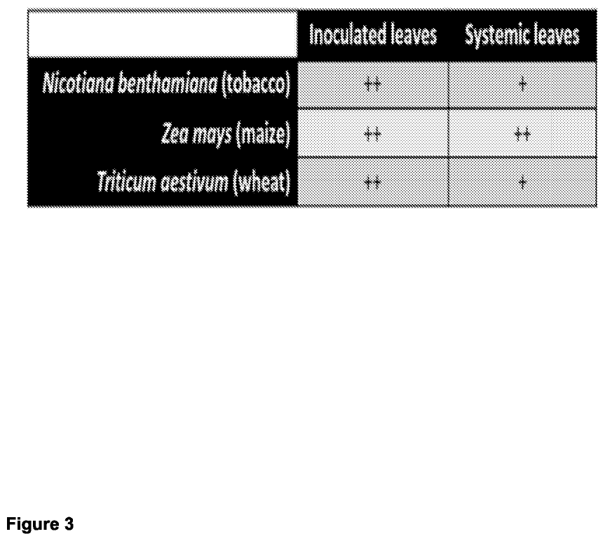

Gene expressing areas in inoculated leaves were numerous and nearly uniformly spread along the leaf width and/or length in Nicotiana benthamiana (tobacco), Zea mays (Maize) and Triticum aestivum (wheat) (FIG. 3).

Gene expressing areas are limited in size and number in Nicotiana benthamiana (tobacco) and Triticum aestivum (wheat).

Gene expressing areas in systemic leaves were numerous and nearly uniformly spread along the leaf width and/or length in Zea mays (Maize).

### Example 5: Heterologous Expression of Green Fluorescent Protein (GFP) in N. benthamiana Using the Modified FoMV Vectors

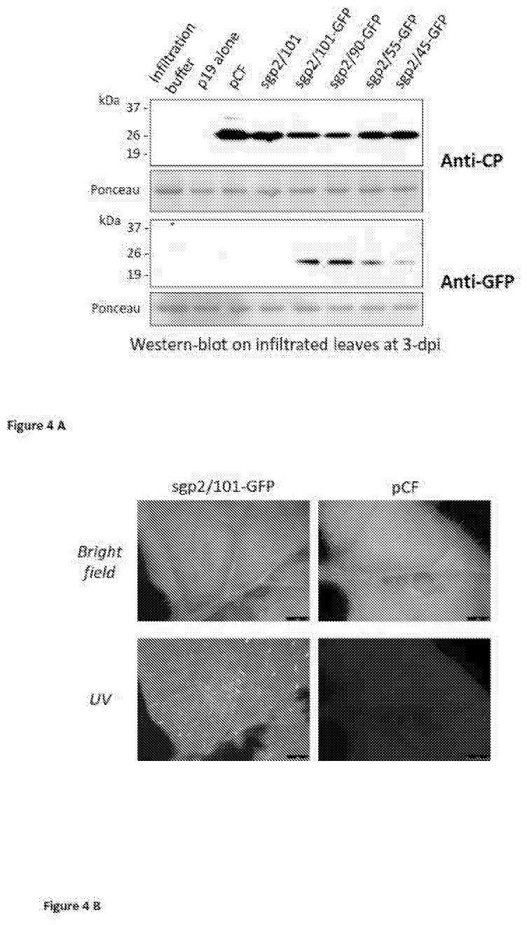

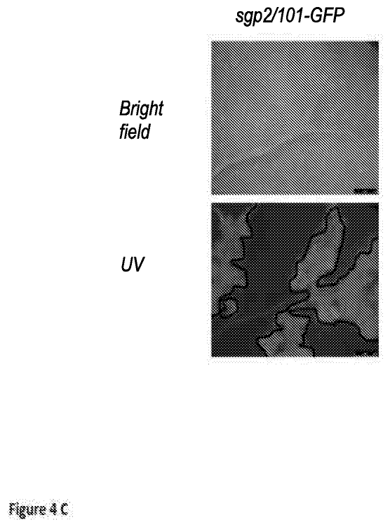

FoMV vectors were delivered to the leaves of N. benthamiana as described in the materials and methods (C.1.). FIG. 4 shows heterologous expression of Green Fluorescent Protein (GFP) in N. benthamiana using the modified FoMV vectors. Panel A shows Immuno-detection of GFP and FoMV coat protein (CP) by Western-blotting from infiltrated leaf samples collected 3 days post infiltration (dpi). p19 is a suppressor of RNA silencing from Tomato bushy stunt virus, which was coinfiltrated with each vector. pCF is the non-modified virus. sgp2-101 is an empty modified FoMV vector containing a 101 nt-long duplicated sgp2 promoter sequence. sgp2/101-GFP, sgp2/90-GFP, sgp2/55-GFP and sgp2/45-GFP are modified FoMV vectors where GFP is expressed under the control of the duplicated 101-, 90-, 55- or 45-nt long sgp2 promoter sequences, respectively. Ponceau-stained protein gel panels show equal protein loading between the samples. Panel B shows sections of infiltrated leaves observed using stereomicroscopy under bright field and UV light at 3-dpi. Panel C shows a section of upper non-infiltrated leaf observed using stereomicroscopy under bright field and UV light at 21-dpi. GFP-expressing areas are delimited by white lines for clarity.

### Example 6: Modified FoMV Vector Sgp2/101-GFP Drives Heterologous Expression of GFP in Maize (Zea mays)

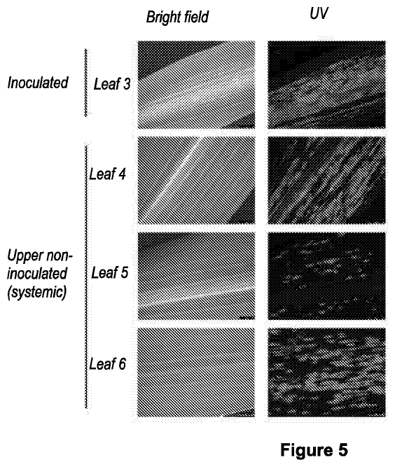

Maize leaves were rub-inoculated with modified FoMV vector sgp2/101-GFP as described in the materials and methods. Sections of rub-inoculated (leaf 3) and upper non-inoculated leaves (leaves 4, 5, 6) were made and GFP fluorescence was observed using stereomicroscopy under bright field and UV light at 13-dpi. Heterologous expression of GFP was observed in maize (Zea mays) using the newly modified FoMV vector sgp2/101-GFP in both inoculated and systemic leaves (FIG. 5).

### Example 7: Modified FoMV Vector Sgp2/101-GFP Drives Heterologous Expression of GFP in Bread Wheat (Triticum aestivum)

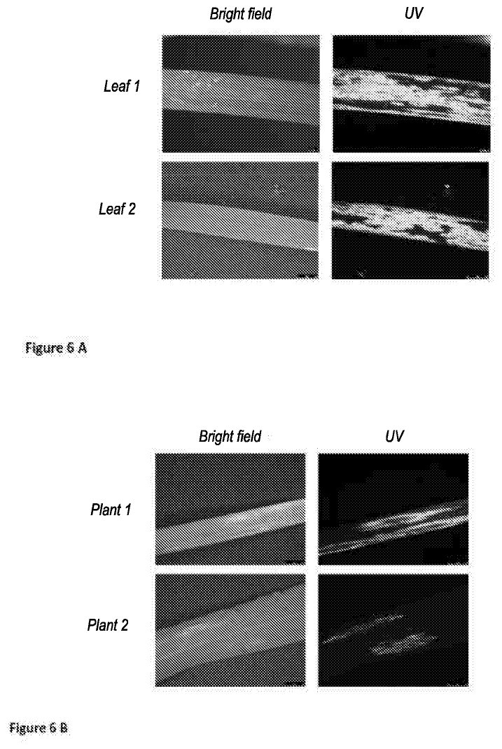

Bread wheat (Triticum aestivum) leaves were rub-inoculated with modified FoMV vector sgp2/101-GFP as described in the materials and methods. Sections of rub-inoculated leaves (leaf 1, 2) were made and GFP fluorescence was observed using stereomicroscopy under bright field and UV light at 8-dpi (FIG. 6A). Sections of upper non-inoculated fourth leaves observed using stereomicroscopy under bright field and UV light at 26-dpi (FIG. 6B). Fourth leaves of two plants (plant 1, 2) are shown. Heterologous expression of GFP was observed in bread wheat (Triticum aestivum) using the newly modified FoMV vector sgp2/101-GFP in both inoculated and systemic leaves (FIG. 6).

### Example 8: Extent of Spread and Level of GFP Expression in Bread Wheat (Triticum aestivum) are Influenced by Growth Conditions

To determine the influence of the growth conditions on the effectiveness of the modified FoMV vector bread wheat (Triticum aestivum) plants which had been rub-inoculated with modified FoMV vector sgp2/101-GFP as described in the materials and methods were grown under two sets of growth conditions and GFP fluorescence as a proxy for gene expression was observed as described previously.

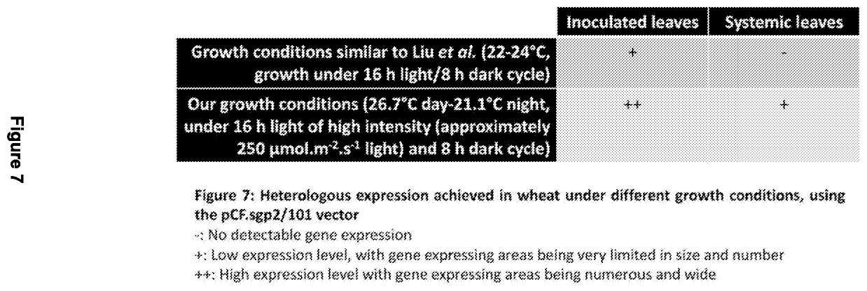

Firstly, growth conditions similar to those described in Liu et al. 2016, Plant Physiology 171:1801-1807 (22-24° C., growth under 16 h light/8 h dark cycle) resulted in low expression level, with gene expressing areas being very limited in size and number in inoculated leaves (+) and no gene expression was detectable (−) in upper non-inoculated (systemic) leaves (FIG. 7).

Secondly, bread wheat (Triticum aestivum) plants which had been rub-inoculated with Modified FoMV vector sgp2/101-GFP as described in the materials and methods were grown under conditions of 26.7° C. day-21.1° C. night, under 16 h light of high intensity (approximately 250 μmol·m-2·s-1 light) and 8 h dark cycle. In contrast to the first conditions, high levels of expression with gene expressing areas being numerous and wide (++) were observed in inoculated leaves and low levels of expression, with gene expressing areas being very limited in size and number (+) were observed in upper non-inoculated (systemic) leaves (FIG. 7).

This result indicates that the optimal conditions for this modified FoMV vector in achieving high-levels of localised expression and in achieving systemic expression are 26.7° C. day-21.1° C. night, under 16 h light of high intensity (approximately 250 μmol·m-2·s-1 light) and 8 h dark cycle.

### Example 9: Modified FoMV Vectors pCF.sgp2/101, PV139wa.sgp2/101 and PV139wa.sgp2/170 Vectors Drive Heterologous Expression of GFP in Bread Wheat (Triticum aestivum)

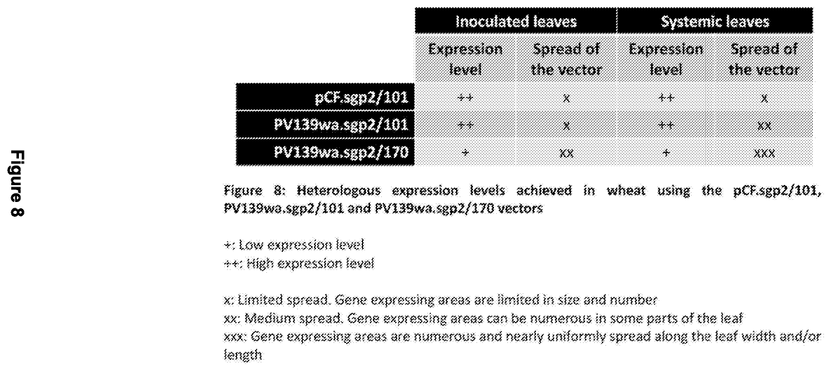

Bread wheat (Triticum aestivum) leaves were independently rub-inoculated with modified FoMV vectors pCF.sgp2/101, PV139wa.sgp2/101 and PV139wa.sgp2/170 as described previously. Sections of rub-inoculated leaves and upper non-inoculated leaves were made and GFP fluorescence was observed using stereomicroscopy under bright field and UV light as described previously. Heterologous expression of GFP was observed in bread wheat (Triticum aestivum) using the newly developed modified FoMV vectors pCF.sgp2/101, PV139wa.sgp2/101 and PV139wa.sgp2/170 in both inoculated and systemic leaves (FIG. 8).

The results were categorised as follows:

+: Low expression level
++: High expression level
x: Limited spread. Gene expressing areas are limited in size and number
xx: Medium spread. Gene expressing areas can be numerous in some parts of the leaf
xxx: Extensive spread. Gene expressing areas are numerous and nearly uniformly spread along the leaf width and/or length

pCF.sgp2/101 showed high expression levels (++) and limited spread (x) in inoculated leaves and systemic leaves. PV139wa.sgp2/101 showed high expression levels (++) and limited spread (x) in inoculated leaves and systemic leaves, limited spread (x) in inoculated leaves and medium spread (xx) in systemic leaves.

PV139wa.sgp2/170 showed low expression levels (+) in both inoculated leaves and systemic leaves and medium spread (xx) in inoculated leaves and extensive spread (xxx) in systemic leaves.

### Example 10: FoMV-Mediated Expression of GFP Using a Long Duplicated sgp2

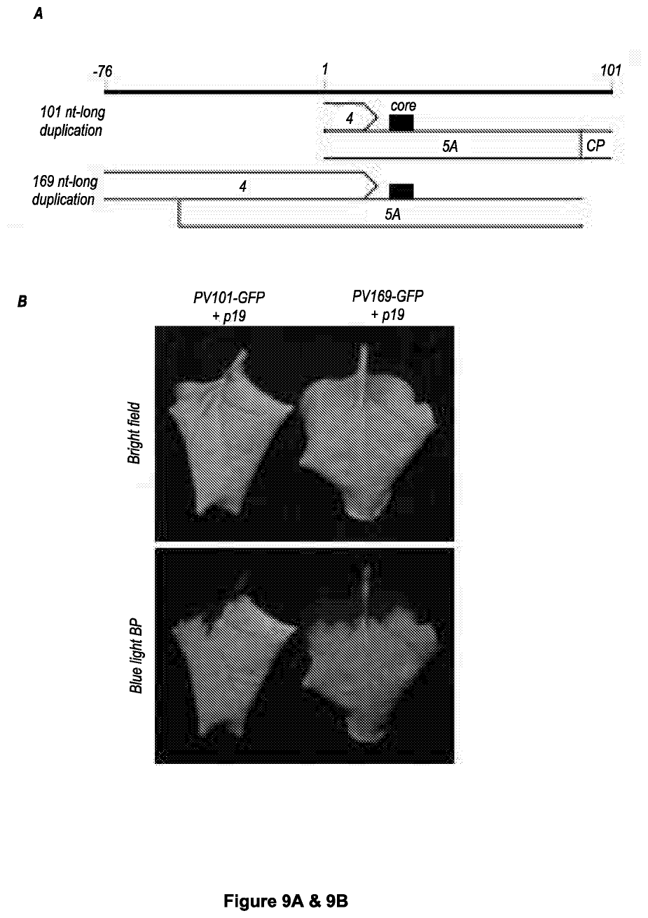

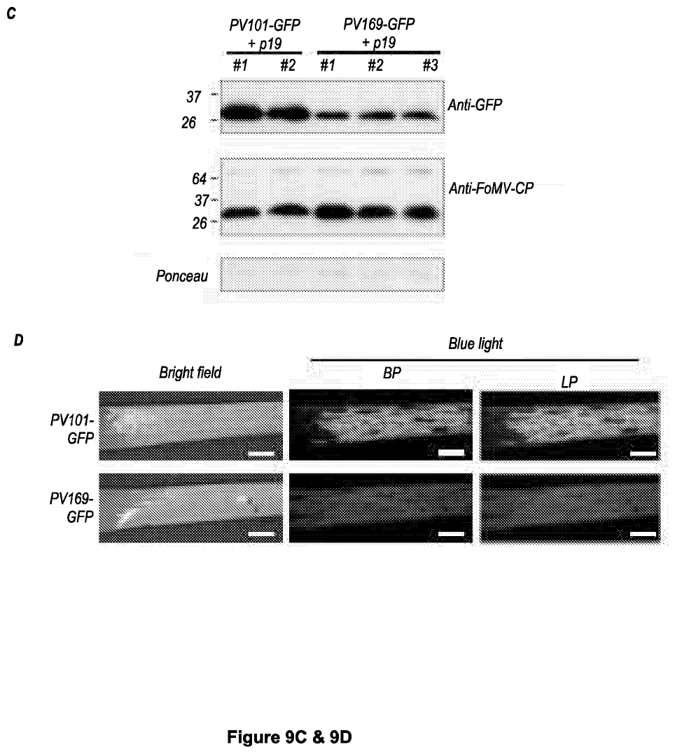

A 169 nt-long sgp2 duplication includes the full 5′-part termini of ORF5A (see FIG. 9A) which is absent in the 101 nt-long duplication (bottom). FIG. 9B shows Agro-infiltrated Nicotiana benthamiana leaves were observed at 7 dpi. Pictures were taken using a camera mounted with a bandpass (BP) filter. FIG. 9C shows immuno-detection of GFP and FoMV-CP in agro-infiltrated leaves of several individual plants by western-blot. Samples were collected at 7 dpi. FIG. 9D shows cv. Riband wheat (Triticum aestivum) leaves rub-inoculated with PV101-GFP or PV169-GFP and observed at 8 dpi using a fluorescence stereomicroscope mounted with bandpass (BP) and longpass (LP) filters. Acquisition settings were kept identical between leaves. White bars represent 2.5 mm. The vector based on pGR-FoMV.PV139 that contains a 169-nt long duplication of sgp2 equivalent to that in the Virus-induced gene silencing vector pFoMV-sg_{[KK1]}. The resulting vector PV169 could express GFP in both Nicotiana benthamiana and wheat (Triticum aestivum), however the observed GFP fluorescence in the infected leaves was less intense than that in the PV101-GFP infected leaves (FIGS. 9B & 9D) and the PV169-GFP-infiltrated Nicotiana benthamiana leaves accumulated less GFP but more viral CP than the PV101-GFP infiltrated leaves as determined by immunoblotting (FIG. 9C).

### Example 11: Second Generation FoMV Expression Vector with Gateway Cassette

In vitro synthesized commercially (Invitrogen) full-length cDNA copy of the FoMV isolate PV139 was cloned into the pGR106 vector backbone between EcoRV and AflII restriction nuclease sites generating the binary plasmid pGR-FoMV.PV139. A full-length FoMV cDNA in this plasmid is flanked at the 5′-end by the CaMV 35S promoter and at the 3′-end by the nos terminator sequence. This pGR-FoMV.PV139 was used for developing a second generation FoMV expression vector using the same methodology as described above for pCF.sgp2/101. A 101-nt long fragment spanning the core FoMV SGP2 sequence was duplicated and a MCS containing recognition sites for NotI, ClaI, AscI, HpaI, and XbaI was inserted downstream of the first sgp2 copy. The resulting vector was named PV101. PV101 was modified by replacing the multiple cloning site with the Gateway® cassette to produce a second, prototype vector, PV101gw, that allows insertion of heterologous sequences using recombination-based cloning. In more detail, a first PCR (PCR1) was done using pGR-FoMV.PV139 as the template and the oligonucleotides PVsorg-6F and PV101gw-R1. A Gateway® cassette was amplified by PCR (PCR2) from the vector pGWB605 (Nakamura et al., 2010 Bioscience Biotechnology and Biochemistry 74: 1315-1319) using the oligonucleotides PV101gw-F2′ and PV101gw-R2′. A third amplicon (PCR3) was produced from pGR-FoMV.PV139 with the oligonucleotides PV101gw-F3 and PVsorg-8R. Amplicons from PCR1, PCR2 and PCR3 were assembled into SpeI plus AvrII digested pGR-FoMV.PV139 using the NEBuilder HiFi DNA assembly system to obtain the vector PV101gw. Both empty vectors made were infectious.

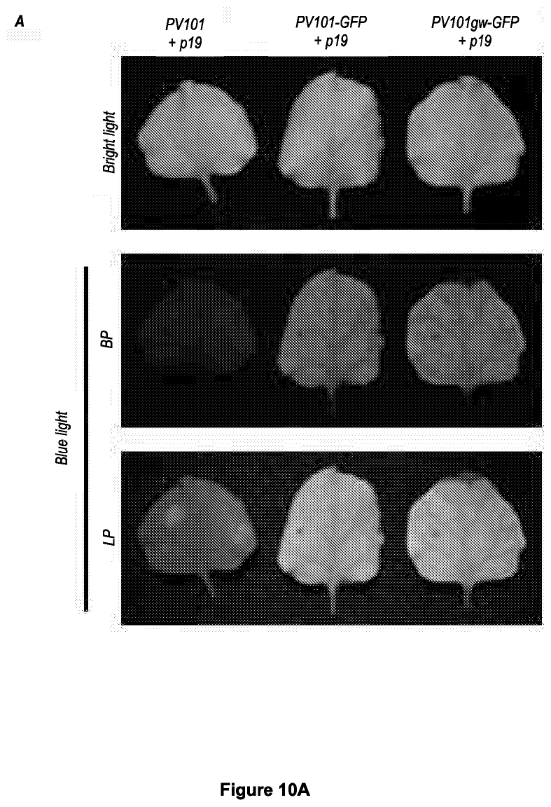

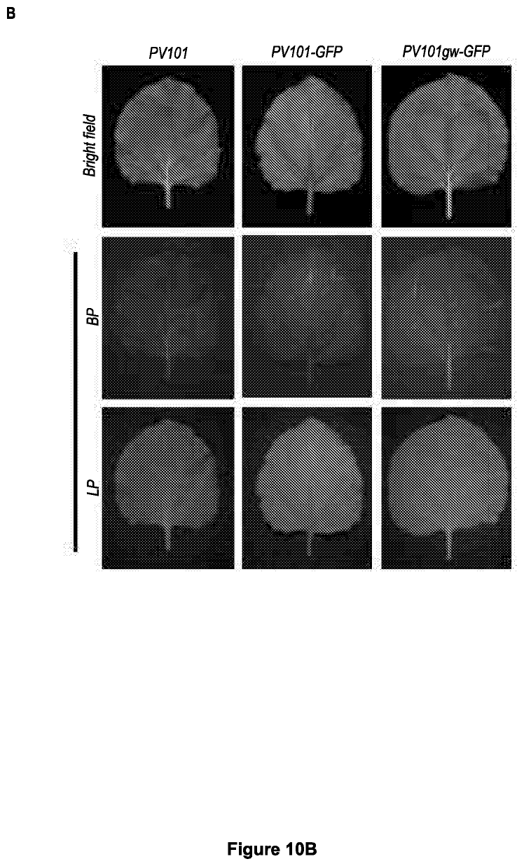

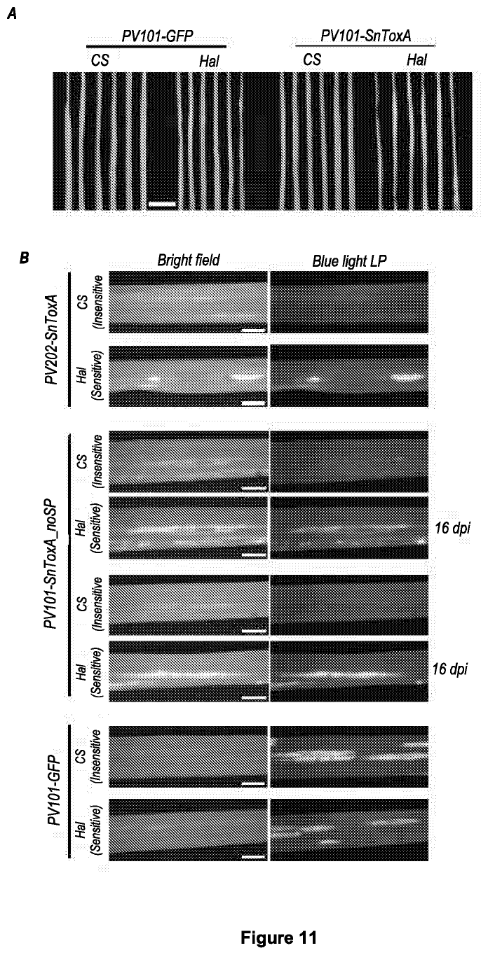

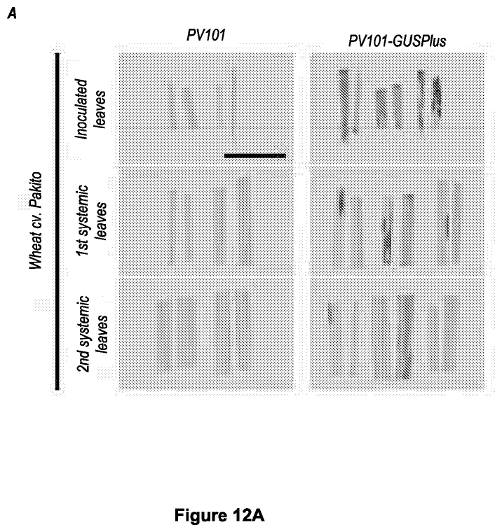

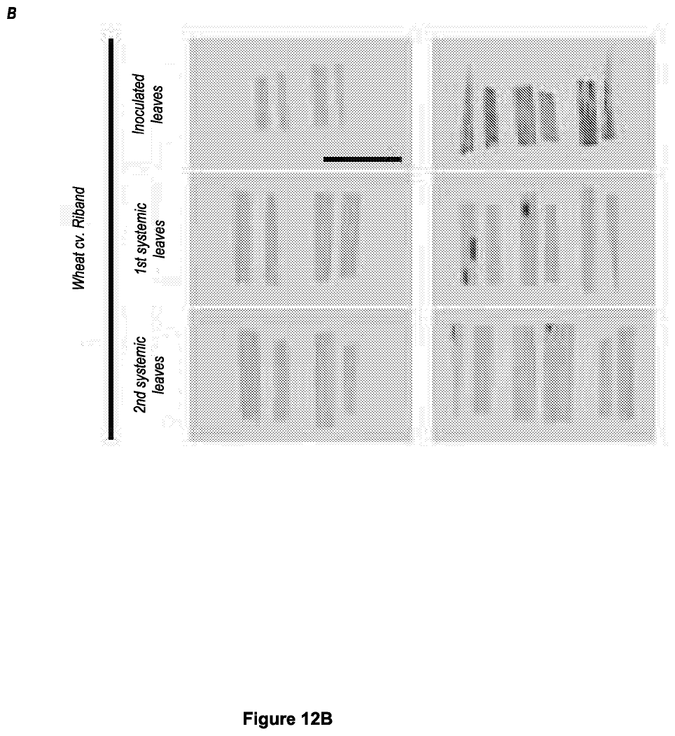

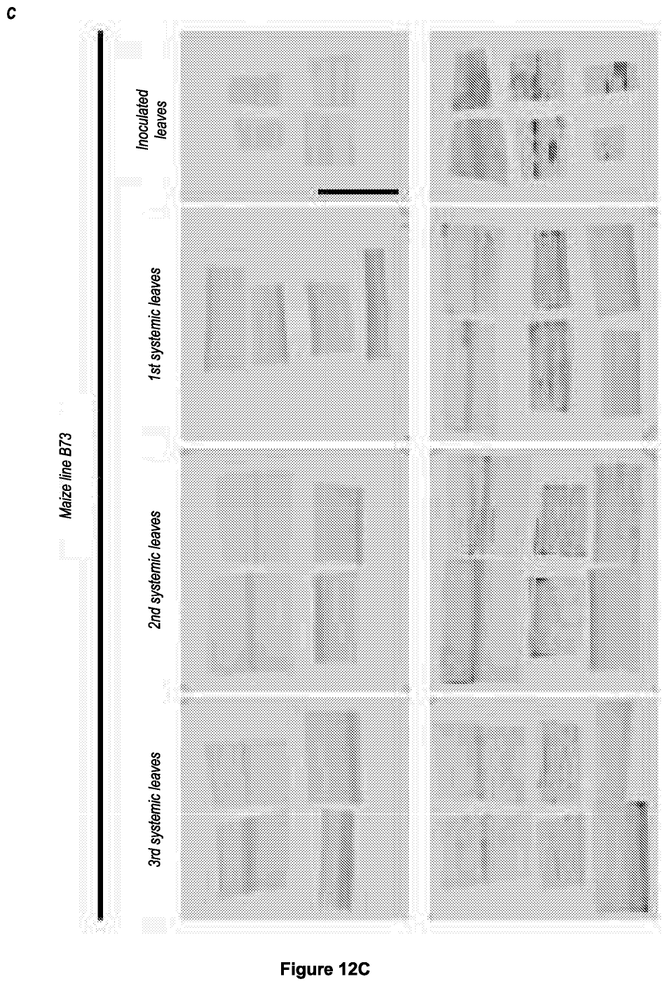

An expression construct produced by recombining coding sequence of gfp (S65T) into the Gateway-enabled vector PV101gw, was also fully infectious and GFP expression was detected in the infected plant tissues using a handheld Dual Fluorescent Protein flashlight (Nightsea). FIG. 10A shows agroinfiltrated leaves at 6 dpi. Leaves were co-agroinfiltrated with an Agrobacterium tumefaciens strain carrying a binary vector coding for the suppressor of silencing p19. The empty vector PV101 was used as negative control. FIG. 10B shows systemic leaves at 16 dpi. Pictures were taken using a camera mounted with bandpass (BP) and longpass (LP) filters.

### Example 12: Cloning of Genes Encoding Reporter Proteins and a Fungal Necrotrophic Effector Protein into the FoMV Expression Vectors

The coding sequence of the S65T variant of GFP gene was amplified from the plasmid pActIsGFP using the oligonucleotides GFP5′-C/al-fw and GFP3′-XbaI-rev. The corresponding amplicon was digested with C/al plus XbaI and cloned into C/al plus XbaI-digested PV101 to create PV101-GFP. The GFP coding sequence was also amplified from pActIsGFP using the oligonucleotides attB1-GFP-F and attB2-GFP-R, and the obtained amplicon was recombined into the Gateway enabled FoMV vector PV101gw using BP clonase II enzyme mix (Invitrogen) following the manufacturers protocol, to produce PV101gw-GFP. The coding sequence of the Stagonospora nodorum ToxA gene was amplified from the plasmid pDONR207-ToxA+SP-STOP using the oligonucleotides ClaI-SnToxA-F and XbaI-SnToxA-R. The amplicon was then digested using C/al plus XbaI and cloned into C/al plus XbaI-digested PV101 to produce PV101-SnToxA.

The coding sequence of GUSPlus (Jefferson et al., 2002 Microbial beta-glucuronidase genes, gene products and uses thereof. In: Center for the Application of Molecular Biology to International Agriculture, Canberra, Australia) in the plasmid pRRes104.293 served as a template for two PCRs producing partially overlapping amplicons using primers ClaI-woGUS-F1 and woGUS-R1 (PCR 1) and woGUS-F2 and XbaI-woGUS-R2 (PCR 2). The amplicons from PCR1 and PCR2 were then fused together using an additional cycle of PCR and the oligonucleotides ClaI-woGUS-F1 and XbaI-woGUS-R2. The resulting amplicon, containing a GUSPlus coding sequence with the internal C/al recognition site removed, was digested using C/a and XbaI and cloned into C/al plus XbaI-digested PV101 to obtain PV101-GUSPlus.

The FoMV vector PV101 can be used as a tool for expression of pathogen effector proteins. The full-length coding sequence of a well-studied necrotrophic effector ToxA (Friesen et al., 2006; Liu et al., 2006) was cloned from Stagonospora nodorum, the causal agent of glume blotch disease in wheat, into PV101. ToxA is known to induce necrosis on wheat cultivars carrying the corresponding sensitivity gene Tsn1 (Friesen et al., 2006; Faris et al., 2010). Indeed, only ToxA-sensitive wheat (Triticum aestivum) cv. Halberd but not ToxA-insensitive wheat cv. Chinese Spring when inoculated with the PV101-SnToxA construct developed necrosis in both inoculated and systemically infected leaves (FIGS. 9A-B). A mature version of ToxA (without its native signal peptide) was also cloned into PV101. The resulting PV101-SnToxA_noSP induced necrosis in systemic leaves of wheat cv. Halberd only but the necrosis was delayed by at least 5 days in comparison with the necrosis induced by the full length ToxA. This indicates that secretion of ToxA into the apoplastic space is not absolutely required, which agrees well with the previous work by Manning and Cuiffetti (Manning and Ciuffetti, 2005) demonstrating that ToxA is imported within the cell in Tsn1 wheat lines. PV101-GFP was used as a control in these experiments and induced only mild chlorotic mosaics on both cultivars (FIG. 9). The FoMV vector PV101 is therefore is useful for VOX applications such as medium-to-high throughput screens for necrosis or cell-death inducing candidate fungal effectors.

### Example 13: The FoMV Vector PV101 can be Used for Expression of Proteins as Large as 600 Amino Acids

PV101 was tested for in planta expression of proteins of a larger protein. For this experiment, a synthetic gene encoding a modified 600 amino acids long Staphylococcus spp. β-glucuronidase protein GUSPlus (Jefferson et al., 2002) was cloned into PV101. Good levels of GUSPlus expression was observed in the PV101-GUSPlus-inoculated leaves of both wheat and maize seedlings (FIG. 10). However, GUSPlus expression in the systemically infected wheat leaves appeared to be even more limited than expression of GFP (FIG. 10 A-B). This suggests a negative correlation between insert length and vector stability. Marginally higher levels of GUSPlus expression were observed in the upper non-inoculated leaves of wheat cv. Pakito (compare FIGS. 10A and B), which agrees well with the fact that this same cultivar showed the best GFP fluorescence scores in the systemically infected leaves among all wheat cultivars tested. By contrast to the somewhat disappointing performance of PV101-GUSPlus in wheat, good levels of GUSPlus expression were observed in the upper non-inoculated maize leaves. The percentage of symptomatic maize plants showing systemic GUSPlus expression varied from 33% to 100% between different experiments. The best GUSPlus expression levels were observed in L5 with weaker and patchy expression in L4 and L6. The FoMV vector PV101 will express heterologous proteins of up to 600 amino acids in the inoculated leaves of wheat and maize, and in up to three consecutive systemically infected leaves in maize. The FoMV vector PV101 successfully expresses a wide range of native proteins from 178-aa long (SnToxA) to at least ca. 600-aa long (GUSPlus) in both inoculated and in systemically infected leaves of wheat and maize. PV101 can be used for VOX in plants such as Sorghum bicolor, Setaria italica and Setaria viridis, which are natural hosts for FoMV, as well as in several other monocots including important crops such as barley, oat and rye that can be systemically infected with FoMV under laboratory conditions (Paulsen and Niblett, 1977 Phytopathology 67: 346-1351).

Furthermore, FoMV-mediated VOX using PV101 and PV101gw can be used in medium throughput screens and the vector can be modified further to allow rapid restriction endonuclease independent cloning and thereby increase experimental throughput. This opens a wide range of applications where an easy and rapid method of heterologous protein expression in monocots is needed. For example, PV101 may be used in screens for cell-death activity of secreted or cytosolic candidate pathogen effectors in wheat, maize or other monocot crops or model species, or in screens for proteins with putative insecticide or antifungal activities. Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Nucleotide Sequences Referred to Herein:

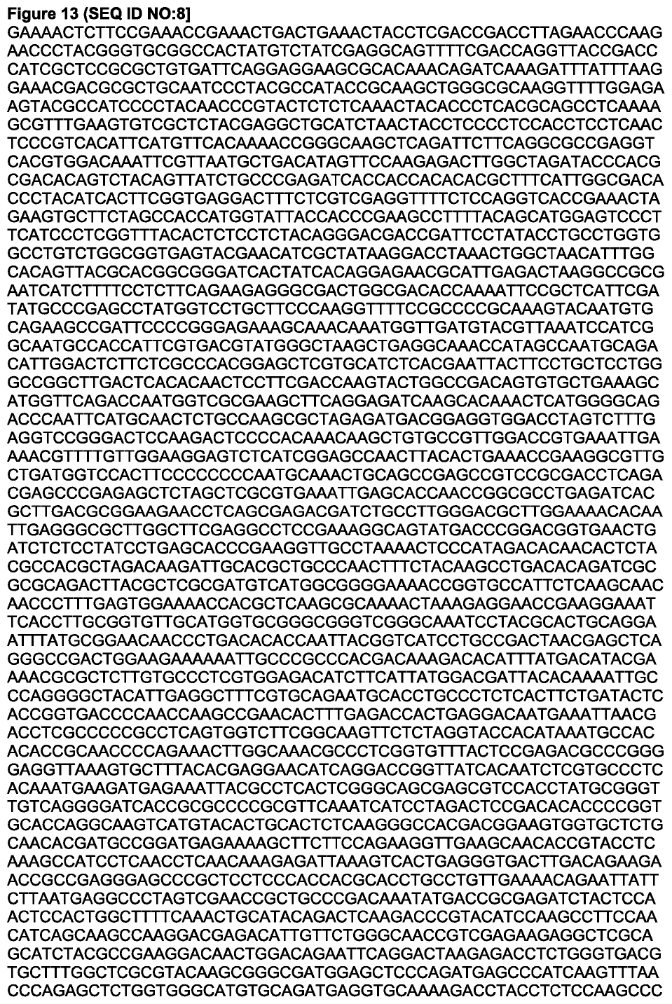

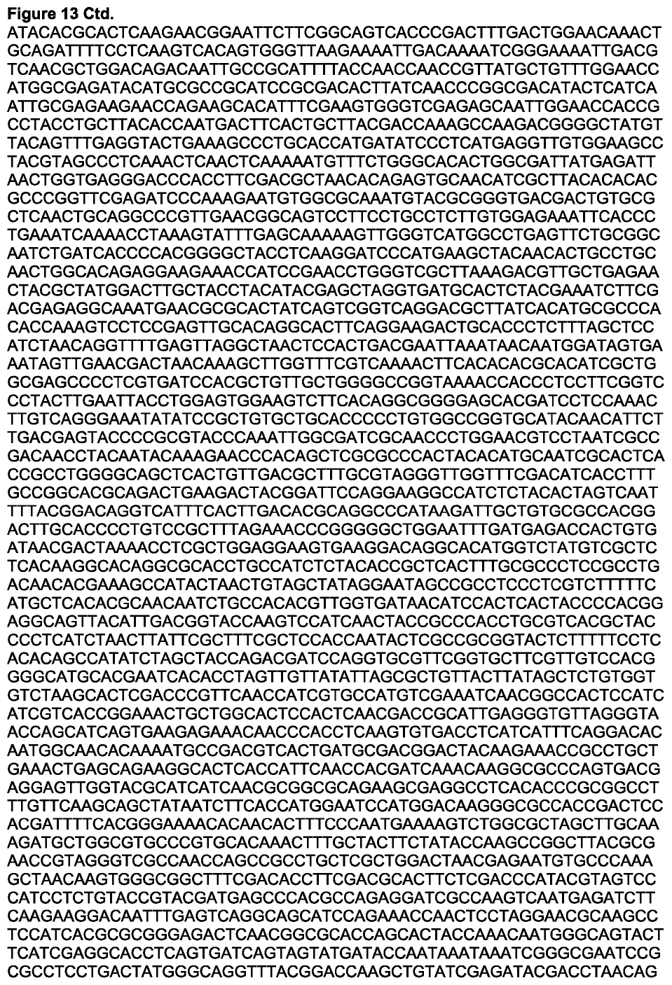

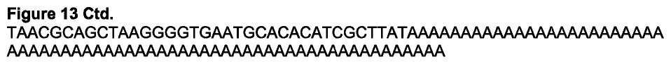

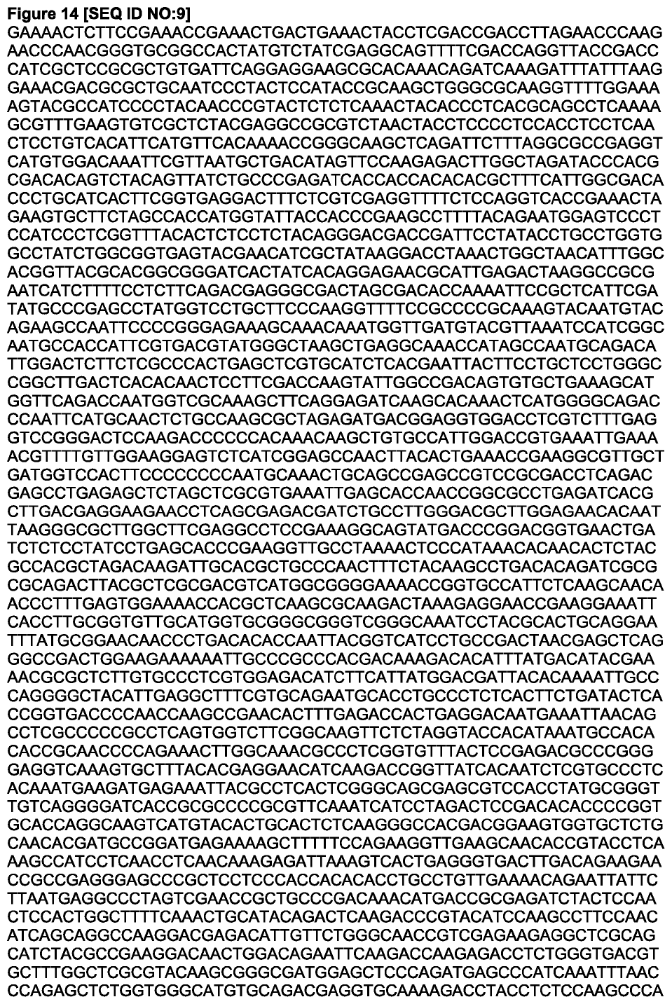

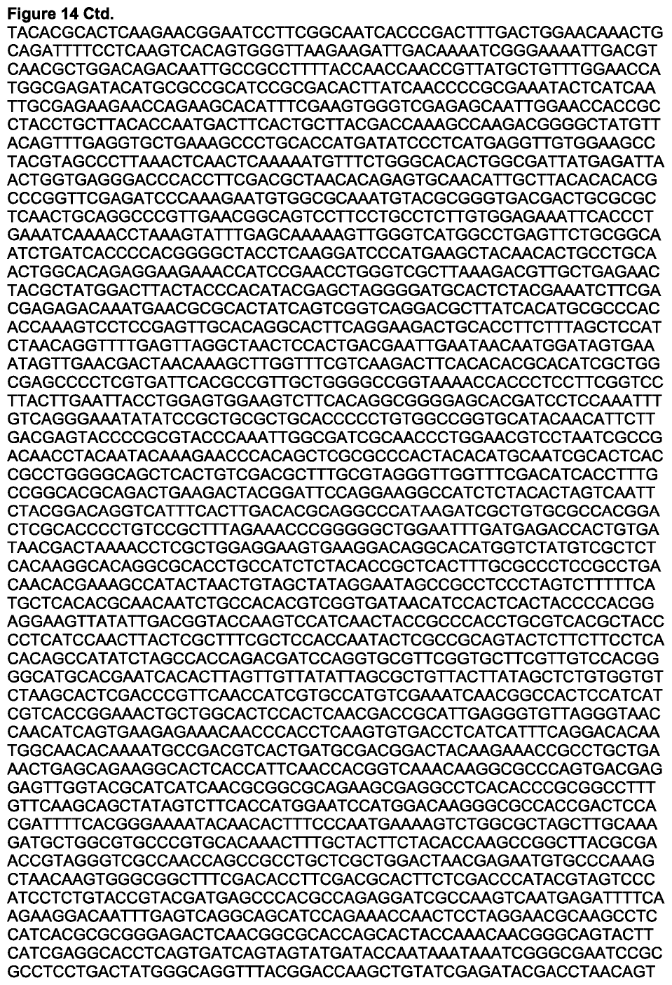

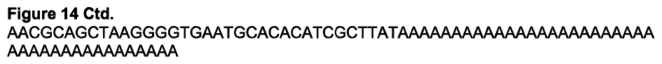

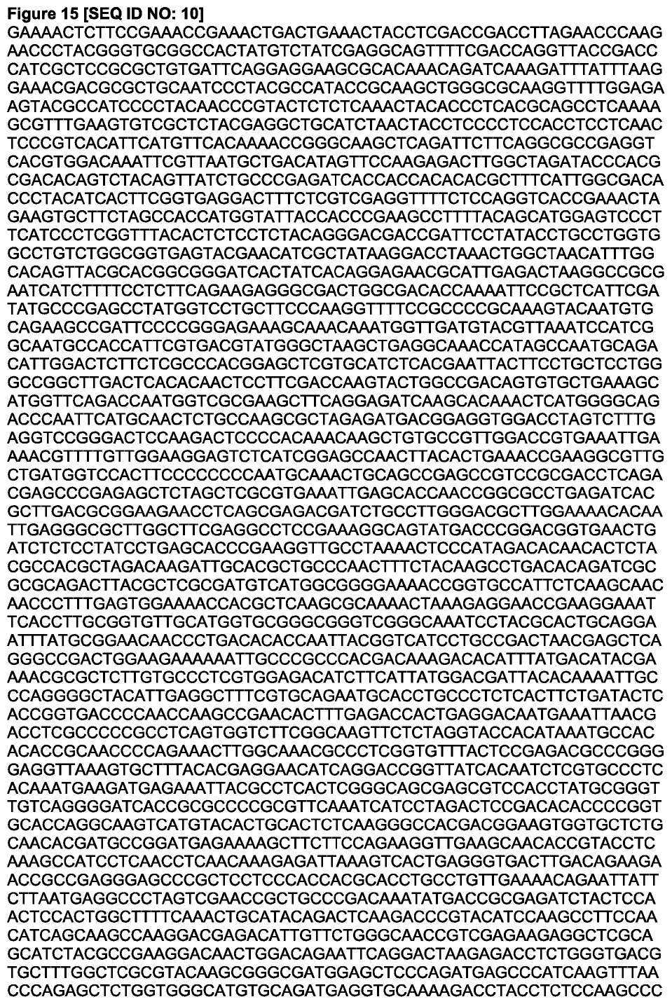

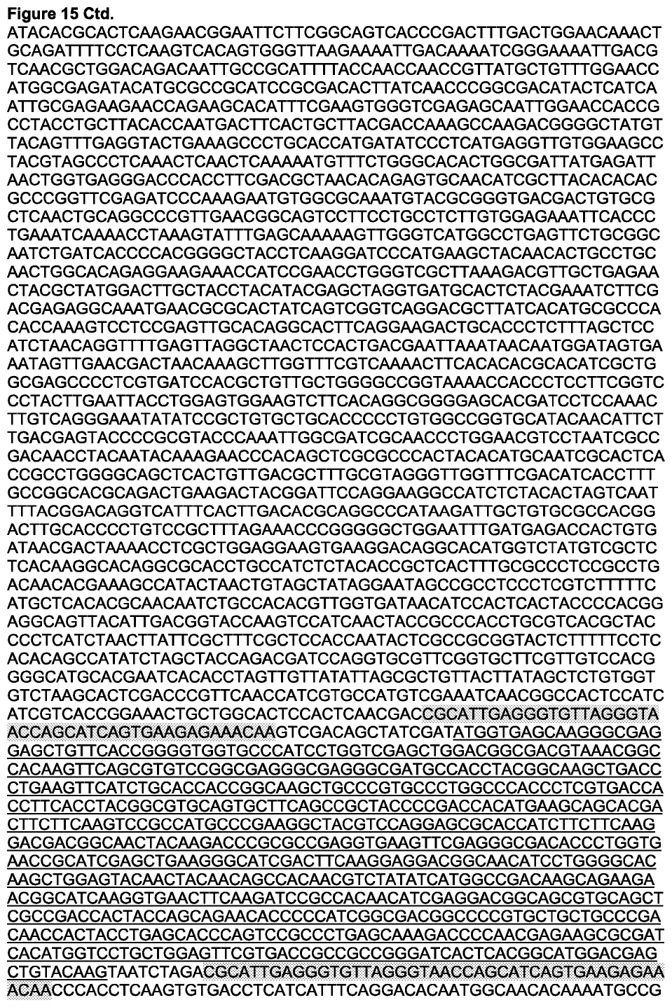

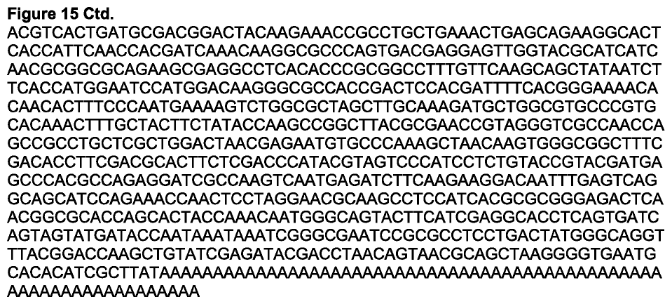

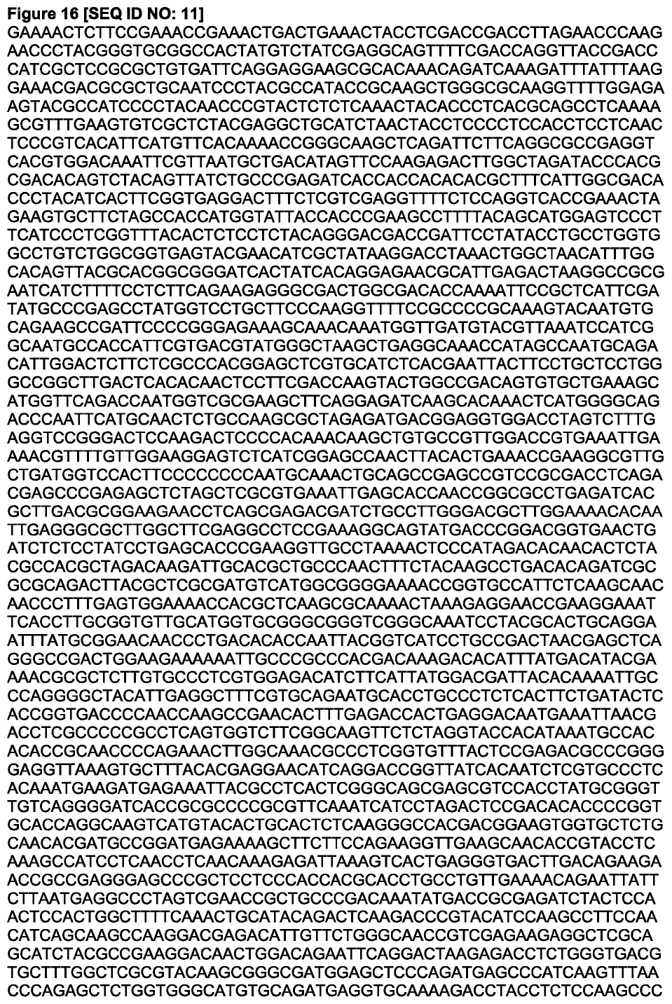

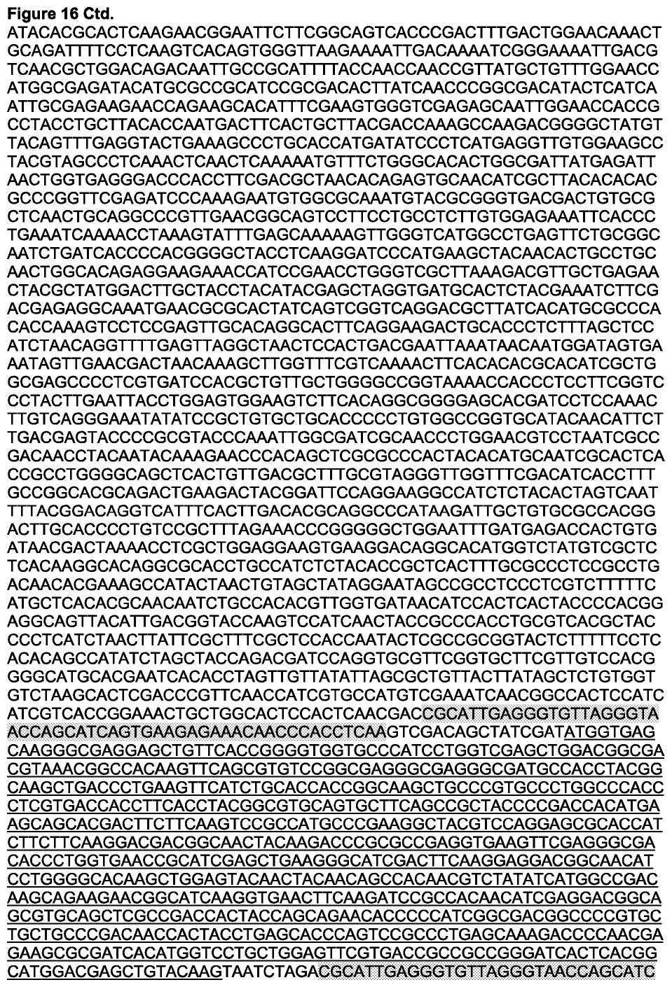

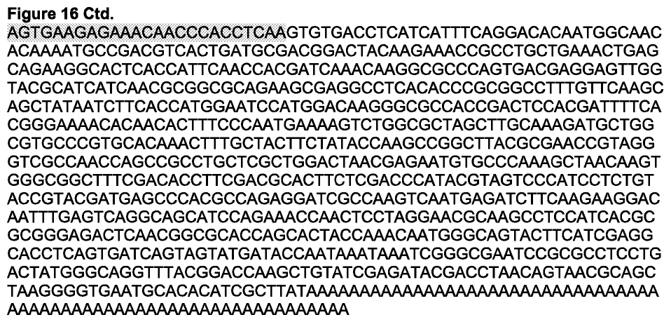

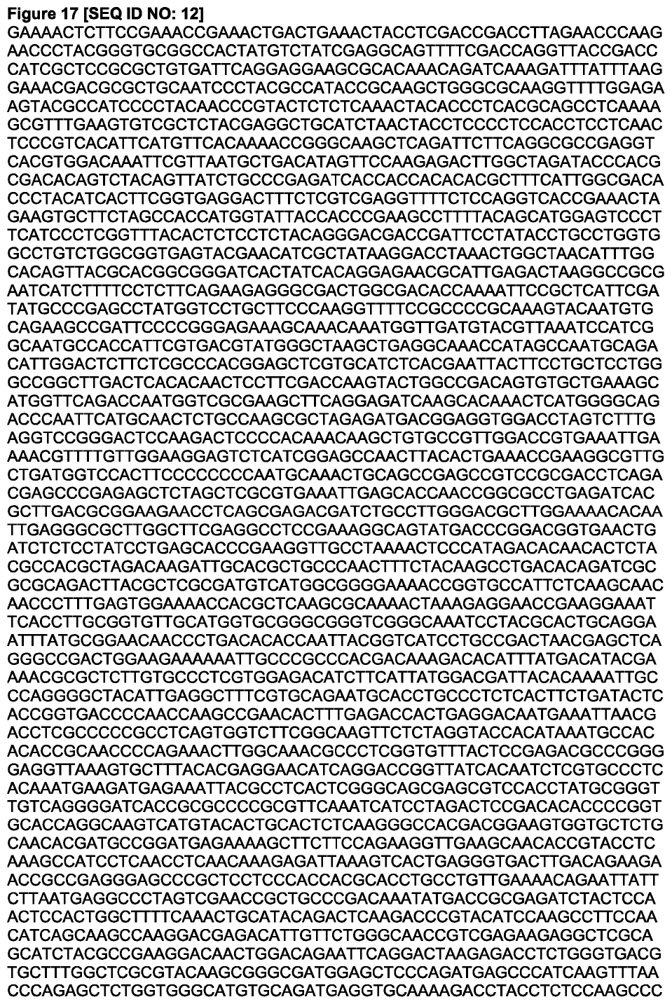

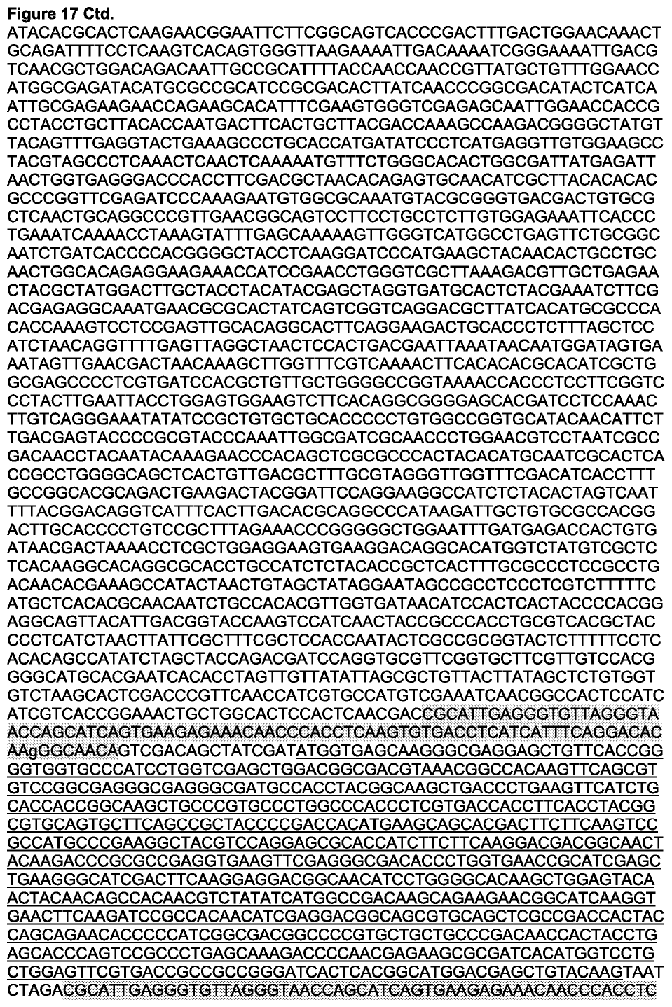

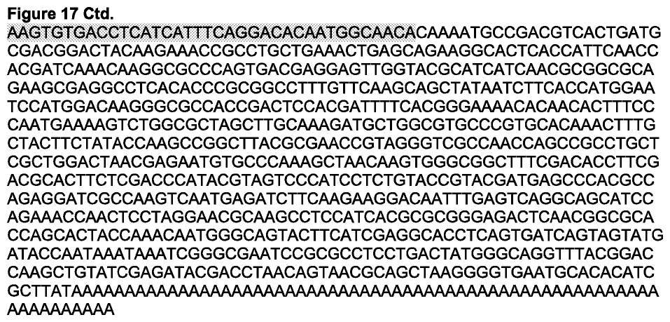

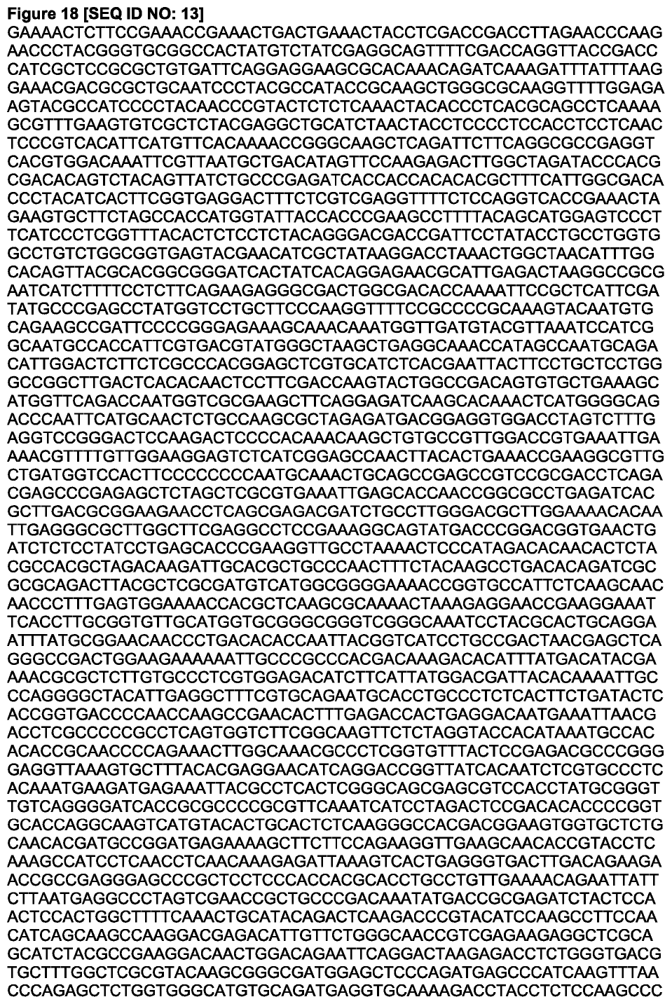

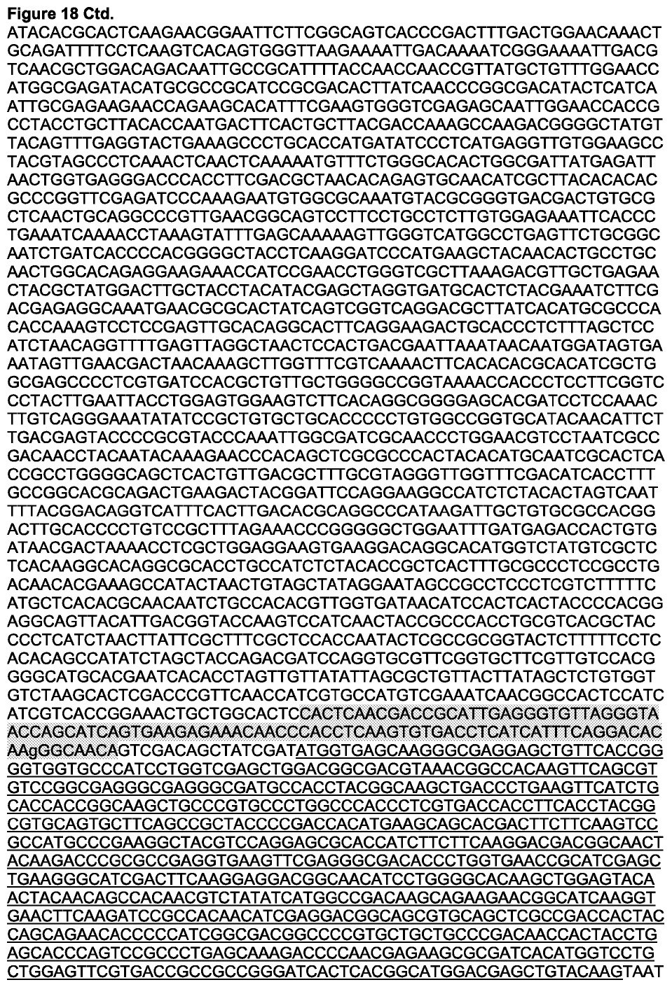

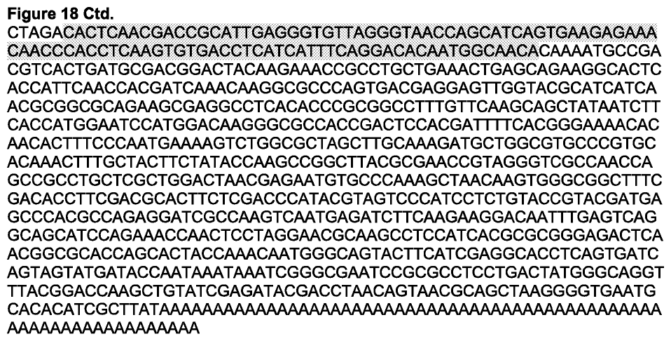

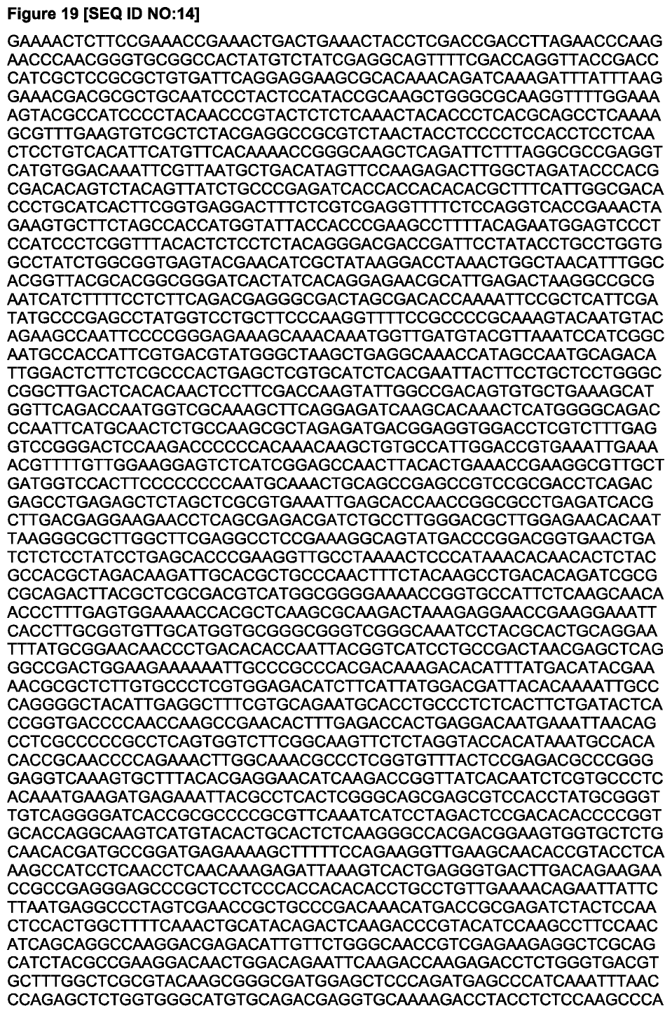

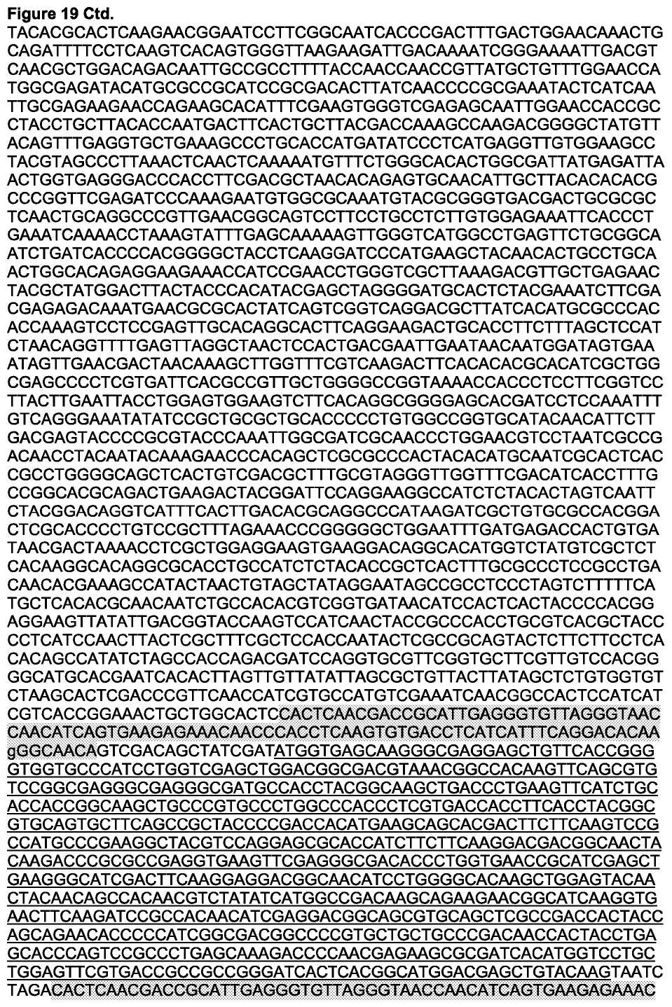

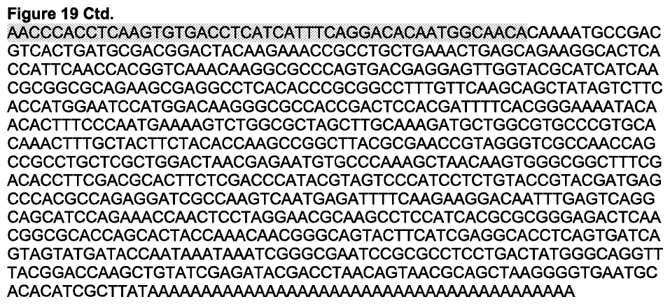

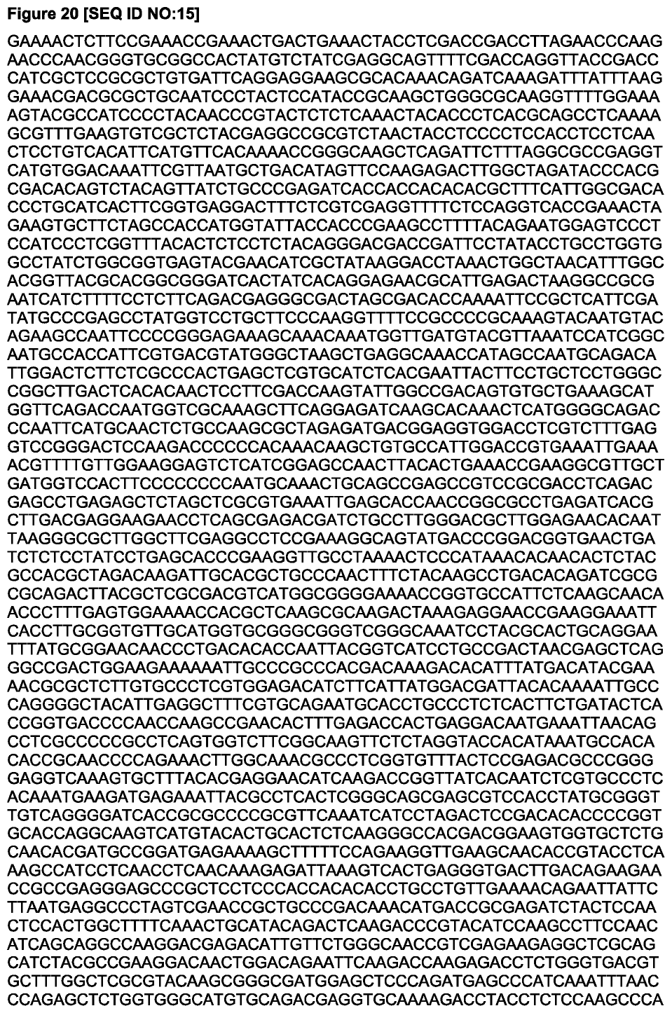

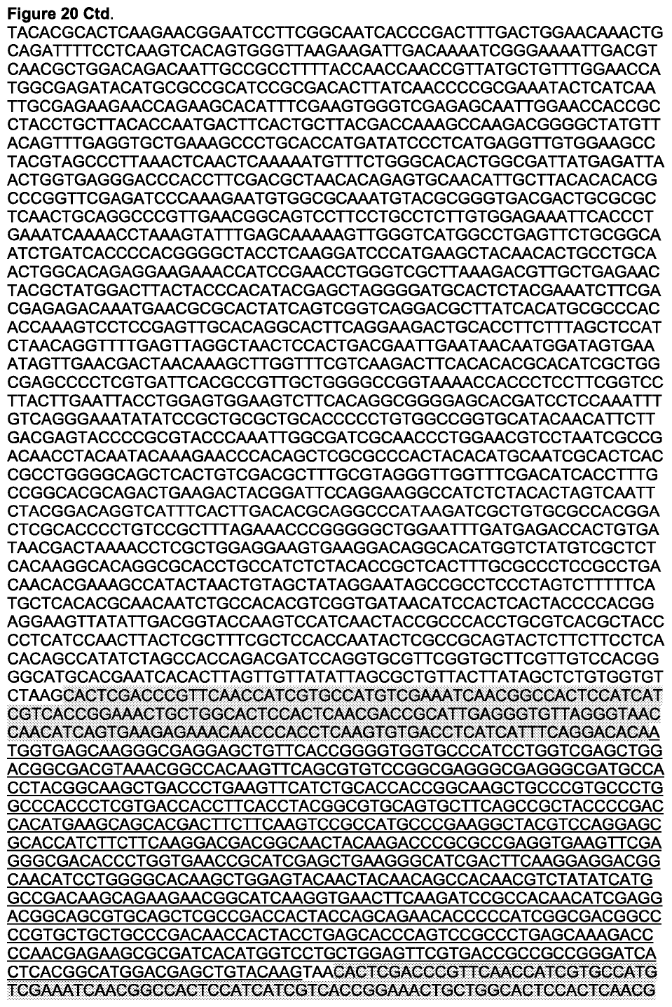

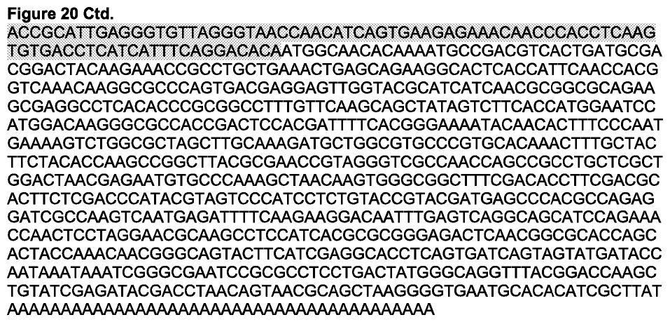

Nucleotide sequence of the variant sgp2/CS of
subgenomic promoter 2:
[SEQ ID NO: 1]
GTTAGGGT
Nucleotide sequence of the variant sgp2/45 of
subgenomic promoter 2:
[SEQ ID NO: 2]
CGCATTGAGGGTGTTAGGGTAACCAGCATCAGTGAAGAGAAACAA
Nucleotide sequence of the variant sgp2/55 of
subgenomic promoter 2:
[SEQ ID NO: 3]
CGCATTGAGGGTGTTAGGGTAACCAGCATCAGTGAAGAGAAACAACCCA
CCTCAA
Nucleotide sequence of the variant sgp2/DS of
subgenomic promoter 2:
[SEQ ID NO: 4]
TGGCAACA
Nucleotide sequence of the variant sgp2/90 of
subgenomic promoter 2:
[SEQ ID NO: 5]
CGCATTGAGGGTGTTAGGGTAACCAGCATCAGTGAAGAGAAACAACCCA
CCTCAAGTGTGACCTCATCATTTCAGGACACAATGGCAACA
Nucleotide sequence of the variant sgp2/101 of
subgenomic promoter 2:
[SEQ ID NO: 6]
CACTCAACGACCGCATTGAGGGTGTTAGGGTAACCAGCATCAGTGAAGA
GAAACAACCCACCTCAAGTGTGACCTCATCATTTCAGGACACAATGGCA
ACA
Nucleotide sequence of the variant sgp2/170 of
subgenomic promoter 2:
[SEQ ID NO: 7]
CACTCGACCCGTTCAACCATCGTGCCATGTCGAAATCAACGGCCACTCC
ATCATCGTCACCGGAAACTGCTGGCACTCCACTCAACGACCGCATTGAG
GGTGTTAGGGTAACCAACATCAGTGAAGAGAAACAACCCACCTCGAGTG
TGACCTCATCATTTCAGGACACA

[SEQ ID NO: 8] Nucleotide sequence of the Taiwanese FoMV isolate pCF, with no modifications (see FIG. 13)
[SEQ ID NO: 9] Nucleotide sequence of the wheat-adapted isolate PV139wa, with no modifications (See FIG. 14):
[SEQ ID NO: 10] Nucleotide sequence of the vector pCF.sgp2/45-GFP where the duplicated sgp2 is 45 nt-long (duplicated regions are in highlighted in grey), and where the GFP coding sequence is underlined (see FIG. 15).
[SEQ ID NO: 11] Nucleotide sequence of the vector pCF.sgp2/55-GFP where the duplicated sgp2 is 55 nt-long (duplicated regions are in highlighted in grey), and where the GFP coding sequence is underlined (See FIG. 16).
[SEQ ID NO: 12] Nucleotide sequence of the vector pCF.sgp2/90-GFP where the duplicated sgp2 is 90 nt-long (duplicated regions are in highlighted in grey), and where the GFP coding sequence is underlined. The start codon which is present in the duplicated region was mutated by a T to G substitution in the first repeat of the duplicated sequence. The mutated nucleotide is in lower case (see FIG. 17).
[SEQ ID NO: 13] Nucleotide sequence of the vector pCF.sgp2/101-GFP where the duplicated sgp2 is 101 nt-long (duplicated regions are in highlighted in grey), and where the GFP coding sequence is underlined. The start codon which is present in the duplicated region was mutated by a T to G substitution (shown in lowercase) in the first repeat of the duplicated sequence (see FIG. 18).
[SEQ ID NO: 14] Nucleotide sequence of the vector PV139wa.sgp2/101-GFP where the duplicated sgp2 is 101 nt-long (duplicated regions are in highlighted in grey), and where the GFP coding sequence is underlined. The start codon which is present in the duplicated region was mutated by a T to G substitution in the first repeat of the duplicated sequence. The mutated nucleotide is in lower case (see FIG. 19).
[SEQ ID NO: 15] Nucleotide sequence of the vector PV139wa.sgp2/170-GFP (170-nt long sgp2 duplication). Duplicated regions highlighted in grey. GFP coding sequence is underlined (see FIG. 20).

5′35Sp
[SEQ ID NO: 16]
CTCGCATGCCTGCAGGTC
5′FoMV-3′35Sp
[SEQ ID NO: 17]
CGGTTTCGGAAGAGTTTTCCCTCTCCAAATGAAATGAA
3′35Sp-5′FoMV
[SEQ ID NO: 18]
TTCATTTCATTTGGAGAGGGAAAACTCTTCCGAAACCG
Spel-FoMV1040R
[SEQ ID NO: 19]
TATCACTAGTGGTTTGCCTCAGCTTAGC
pBxs-fw
[SEQ ID NO: 20]
ATACTCGAGGCGTGGATCCCGCTGGGCCTC
pBxs-rev
[SEQ ID NO: 21]
GTTATACTAGTTTTTTCCTTATGCGGCCTT
pB-Fmcs-10-fw1
[SEQ ID NO: 22]
CAGTTAACTTACTCTAGACACTCAACGACCGCATTGAG
pB-Fmcs-10-rev1
[SEQ ID NO: 23]
GTCGACTTGTTCGGCCGAGTGCCAGCAGTTTCCGGT
pB-Fmcs-10-fw2
[SEQ ID NO: 24]
AGTTGGCGCGCCAATCAGTTAACTTACTCTAGACA
pB-Fmcs-10-rev2
[SEQ ID NO: 25]
ATCGATAGCTGTCGACTTGTTCGGCCGAG
pB-Fmcs-fw1
[SEQ ID NO: 26]
CAGTTAACTTACTCTAGACGCATTGAGGGTGTTAGGG
pB-Fmcs-rev1
[SEQ ID NO: 27]
GTCGACTTGTTCGGCCGTCGTTGAGTGGAGTGCCAG
pB-Fmcs-fw2
[SEQ ID NO: 28]
AGTTGGCGCGCCAATCAGTTAACTTACTCTAGACG
pB-Fmcs-rev2
[SEQ ID NO: 29]
ATCGATAGCTGTCGACTTGTTCGGCCGTC
GFP5′-ClaI-fw
[SEQ ID NO: 30]
AGGTCAATCGATATGGTGAGCAAGGGCGAGG
GFP3′-XbaI-rev
[SEQ ID NO: 31]
TATGCTTCTAGATTACTTGTACAGCTCGTCCATG

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. (canceled)

2. A modified Foxtail Mosaic Virus (FoMV) for expression of a gene of interest (GOI) in a plant, plant part or plant cell, wherein the virus comprises a first sgp2 promoter and at least a second sgp2 promoter; and wherein the GOI is under transcriptional control of the at least second sgp2 promoter.

3. A modified FoMV as claimed in claim 2, wherein the first sgp2 promoter is 5′ of a polynucleotide encoding the virus coat protein (CP) and the at least second sgp2 promoter and polynucleotide encoding the polypeptide of interest are 5′ of the first sgp2 promoter.

4. A modified FoMV as claimed in claim 2, wherein the polynucleotide sequence of the at least second sgp2 promoter comprises a polynucleotide sequence selected from the group consisting of:
(a) a polynucleotide sequence of SEQ ID NO: 1, or a sequence of at least 90% identity thereto;
(b) a polynucleotide sequence of SEQ ID NO: 2, or a sequence of at least 90% identity thereto;
(c) a polynucleotide sequence of SEQ ID NO: 3, or a sequence of at least 90% identity thereto;
(d) a polynucleotide sequence of SEQ ID NO: 4, or a sequence of at least 98% identity thereto;
(e) a polynucleotide sequence of SEQ ID NO: 5, or a sequence of at least 90% identity thereto;
(f) a polynucleotide sequence of SEQ ID NO: 6, or a sequence of at least 90% identity thereto; and
(g) a polynucleotide sequence of SEQ ID NO: 7, or a sequence of at least 90% identity thereto.

5.-6. (canceled)

7. A modified FoMV as claimed in claim 2, wherein the virus comprises the sequence selected from the group consisting of:
(a) SEQ ID NO: 8, or a sequence of at least 80% identity thereto; and
(b) SEQ ID NO: 9, or a sequence of at least 80% identity thereto.

8. (canceled)

9. A modified FoMV as claimed in claim 2, wherein the modified FoMV is in the form of a vector; preferably a DNA viral vector or an RNA viral vector.

10. A modified FoMV as claimed in claim 2, wherein the GOI encodes a sequence selected from the group consisting of:
(a) a polypeptide; preferably a heterologous polypeptide; more preferably a polypeptide of microbial, plant or animal/human origin, wherein the polypeptide is at least 200 amino acids in length; and
(b) a protein non-coding sequence, optionally wherein the protein non-coding sequence is selected from the group consisting of a microRNA (miRNA) or a long non-coding RNA (IncRNA).

11-14. (canceled)

15. A Foxtail Mosaic Virus (FoMV) DNA expression construct comprising from 5′ to 3′ polynucleotide sequences encoding a strong heterologous promoter that is active in plants, followed by the viral ORF1, sgp1 promoter, ORF2, ORF3, ORF 4, at least two sgp2 promoters, coat protein (CP) and nopaline synthase terminator (nos), wherein the ORF2 overlaps with ORF3 and ORF3 overlaps with ORF4, and ORF4 includes the start codon of ORF5A, wherein the polynucleotide sequence comprised in or between the duplicated sgp2 promoters includes insertion site(s) for a gene of interest (GOI).

16. An FoMV expression construct as claimed in claim 15, wherein the duplicated portion of the sgp2 promoter has a polynucleotide sequence comprises:
(a) a polynucleotide sequence of SEQ ID NO: 2 or a sequence of at least 90% identity thereto; or
(b) a polynucleotide sequence of SEQ ID NO: 3 or a sequence of at least 90% identity thereto; or
(c) a polynucleotide sequence of SEQ ID NO: 4; or
(d) a polynucleotide sequence of SEQ ID NO: 5 or a sequence of at least 90% identity thereto; or
(e) a polynucleotide of SEQ ID NO: 6 or a sequence of at least 90% identity thereto.

17. An FoMV expression construct as claimed in claim 15, wherein the insertion site comprises at least one selected from the group consisting of:
(a) restriction sites; optionally wherein the restriction sites are Sal\-Cla\-Asc\-Hpa\-Xba\; or Not\-Cla\-Asc\-Hpa\-Xba\;
(b) att recombination cloning sites; optionally wherein the att recombination site is at least one of att or attR; and
(c) a Gateway cassette is inserted between the sgp2 promoters.

18. (canceled)

19. An FoMV expression construct as claimed in claim 15, further comprising a GOI under the control of the duplicated sgp2 promoter.

20. FoMV viral genomic RNA (gRNA) encoded by an FoMV expression construct of claim 19.

21. A method of expressing a protein in a plant, plant part or plant cell, comprising infecting the plant, plant part or a plant cell with a modified FoMV of claim 2.

22. A method as claimed in claim 21, wherein the plant is a monocotyledonous plant, or the plant part or plant cell is of a monocotyledonous plant; preferably a plant selected from the group consisting of Triticum sp. and Zea sp.; more preferably Triticum aestivum.

23. A method as claimed in claim 21, wherein the modified FoMV is in the form of an FoMV expression construct comprising from 5′ to 3′ polynucleotide sequences encoding a strong heterologous promoter that is active in plants, followed by the viral ORF1, sgp1 promoter, ORF2, ORF3, ORF 4, at least two sgp2 promoters, coat protein (CP) and nopaline synthase terminator (nos), wherein the ORF2 overlaps with ORF3 and ORF3 overlaps with ORF4, and ORF4 includes the start codon of ORF5A, wherein the polynucleotide sequence further comprises a GOI, under the control of the duplicated sgp2 promoter, wherein the FoMV expression construct is transformed into Agrobacterium sp. and further wherein a dicotyledonous plant, or a plant part or plant cell of a dicotyledonous plant is inoculated with the Agrobacterium; preferably wherein the dicotyledenous plant is Nicotiana sp.; more preferably Nicotiana benthamiana, or parts or cells thereof; optionally further comprising infecting a monocotyledonous plant with modified FoMV virus particles or FoMV RNA obtained from the Agrobacterium infected diclotyledonous plant.

24. (canceled)

25. A method of transient local or systemic overexpression of a protein in a plant comprising infecting or transfecting the plant with a modified FoMV virus or viral vector of claim 2, and afterwards growing the plant, wherein the infection or transfection is carried out under a first set of environmental conditions and the growing of the plant is carried out under a second set of environmental conditions; optionally wherein the second set of environmental conditions comprises a light/dark photoperiod, with a light temperature of greater than 21° C. and/or a dark temperature of greater than 24° C.; optionally wherein the light intensity is in the range 200 μmol·m⁻²·s¹ to 2500 μmol·m⁻²·s⁻¹.

26.-27. (canceled)

28. A plant, plant part or plant cell infected with a modified FoMV as defined in claim 2, wherein the expressed GOI is selected from the group consisting of:
(a) a protein or polypeptide at least 200 amino acids in length;
(b) a protein non-coding sequence; optionally a sequence encoding a microRNA (miRNA) or long non-coding RNA (IncRNA)
(c) a fungal protein; preferably a fungal effector protein; and
(d) a protein with insecticidal or fungicidal activity.

29-32. (canceled)

33. A plant, plant part or plant cell as claimed in claim 28, wherein the plant, plant part or plant cell has an increased level of expression of a desired GOI compared to a genetically equivalent but uninfected control plant, plant part or plant cell; optionally wherein the plant is a monocotyledonous plant, or the plant part or plant cell is of a monocotyledonous plant preferably Triticum sp. or Zea sp.; more preferably Triticum aestivum.

34. (canceled)

35. A composition comprising a modified FoMV virus particle, as defined in claim 2.

36. A composition as claimed in claim 35 which is an extract of or sap from a plant infected with a modified FoMV.

37. A method of identifying monocotyledonous plants for suitability for a breeding process, comprising infecting a monocot plant with a modified FoMV virus as defined in claim 2, growing the plant for a period of time and then examining the plant for the presence or absence of one or more phenotypic, biochemical and/or genetic characteristics as a measure of said suitability.
